(19) United States
(12) Patent Application Publication
(10) Pub. No.: US 2025/0186372 A1
(43) Pub. Date: Jun. 12, 2025

(54) FLEXIBLE INTERCONNECTED MICROPARTICLES ARRAYS: DEVICES, METHODS, COMPOSITIONS AND APPLICATIONS

(71) Applicant: Pathak Holdings LLC, Phoenix, AZ (US)

(72) Inventor: Chandrashekhar P. Pathak, Phoenix, AZ (US)

(21) Appl. No.: 19/056,429

(22) Filed: Feb. 18, 2025

Related U.S. Application Data
(63) Continuation-in-part of application No. 18/620,289, filed on Mar. 28, 2024.
Application No. 18/620,289 is a continuation-in-part of application No. 17/503,063, filed on Oct. 15, 2021, now abandoned.
Application No. 17/503,063 is a continuation-in-part of application No. 17/324,738, filed on May 19, 2021, now Pat. No. 11,969,398.
Application No. 17/324,738 is a continuation of application No. 16/818,944, filed on Mar. 13, 2020, now Pat. No. 11,045,433.
Application No. 16/818,944 is a continuation of application No. 16/156,949, filed on Oct. 10, 2018, now Pat. No. 10,624,865.
(60) Provisional application No. 63/555,093, filed on Feb. 18, 2024.
(60) Provisional application No. 63/742,125, filed on Jan. 6, 2025.
(60) Provisional application No. 63/744,312, filed on Jan. 12, 2025.

(51) Int. Cl. A61K 31/155; A61K 9/00; A61K 9/06; A61K 9/16; A61K 9/50; A61K 31/337; A61K 31/37; A61K 31/496; A61K 31/573; A61K 31/7036; A61K 31/727; A61K 41/00; A61K 47/02; A61K 47/32; A61K 47/34; A61L 27/22; A61L 27/38; A61L 27/50; A61L 27/54; A61M 5/30; A61M 5/32; A61M 37/00

(52) U.S. Cl. CPC A61K 31/155; A61K 9/0014; A61K 9/0021; A61K 9/0024; A61K 9/0048; A61K 9/0051; A61K 9/06; A61K 9/1641; A61K 9/1647; A61K 9/1658; A61K 9/5031; A61K 9/5052; A61K 31/337; A61K 31/37; A61K 31/496; A61K 31/573; A61K 31/7036; A61K 31/727; A61K 41/0028; A61K 47/02; A61K 47/32; A61K 47/34; A61L 27/225; A61L 27/3804; A61L 27/50; A61M 5/3298; A61M 37/0015; A61M 37/0076; A61L 27/54; A61L 2300/442; A61L 2400/06; A61M 5/3015; A61M 2037/0023; A61M 2037/003; A61M 2037/0046; A61M 2037/0053; A61M 2205/36; A61M 2205/3606; A61M 2205/3633; A61M 2205/3653; A61M 2207/00; C12Y 304/21068; C12Y 304/24069

(57) ABSTRACT

This invention pertains to an advanced implantable surgical array system, wherein array elements, composed of hydrogels or organogels, are intricately connected by a flexible thread. These elements are uniquely embedded within the device through a novel method that involves casting the hydrogel or organogel directly around the thread from a liquid/fluid precursor solution, which upon drying or cross-linking, integrates the thread into the array elements without the necessity for traditional threading techniques. The disclosed arrays are versatile, capable of being configured into various structural forms including single particle arrays, chain-like arrays, as well as two-dimensional and three-dimensional matrices. This innovative approach not only facilitates controlled drug delivery over extended periods but also incorporates features to prevent element migration and enables the device to assume expanded or compressed configurations for ease of implantation.

### CROSS-REFERENCE TO RELATED APPLICATIONS

This application is a continuation-in-part of U.S. patent application Ser. No. 18/620,289 filed Mar. 28, 2024, which is a continuation-in-part of U.S. patent application Ser. No. 17/503,063 filed Oct. 15, 2021, and which is a continuation-in-part of U.S. patent application Ser. No. 17/324,738 filed May 19, 2021, which is a continuation of U.S. patent application Ser. No. 16/818,944 filed Mar. 13, 2020 now U.S. Pat. No. 11,045,433, which is a continuation of U.S. patent application Ser. No. 16/156,949 filed Oct. 10, 2018 now U.S. Pat. No. 10,624,865.

This application also claims priority to the U.S. Provisional Application No. 63/555,093 filed on Feb. 18, 2024, and also claims priority to U.S. Provisional Application No. 63/742,125 filed on Jan. 6, 2025, and also claims priority to U.S. Provisional Application No. 63/744,312 filed on Jan. 12, 2025, which applications are incorporated herein by specific reference in their entirety.

### FIELD OF THE INVENTION

The present invention is generally related to the compositions and methods of biomedical devices and controlled drug delivery systems.

### BACKGROUND OF THE INVENTION

In the realm of controlled drug delivery, microspheres have gained widespread adoption due to their capacity to encapsulate and gradually release therapeutic agents. These microspheres are typically administered as a suspension through subcutaneous or intramuscular injections.

However, a significant drawback of this method is the post-injection behavior of microspheres. They tend to cluster or form shapeless masses, leading to uneven exposure to surrounding tissue fluids. This aggregation reduces the effective surface area for interaction and it can potentially result in unpredictable drug release patterns. Consequently, there is a pressing need for advancements that arrange microspheres in an organized manner, such as an array configuration, to ensure uniform exposure to tissue fluids and consistent release kinetics.

A notable limitation of conventional microsphere-based drug delivery systems is their inability to be retrieved once deployed. Due to their small size and random dispersion within the tissue, microspheres are difficult, if not impossible, to recover post-administration. This limitation poses challenges in scenarios where the removal of the drug delivery system is necessary, such as in cases of adverse reactions, dosage adjustments, or the cessation of therapy. It will be valuable contribution to art wherein a retrievable microspheres drug delivery system is developed.

Researchers have also explored surgical sutures as potential carriers for controlled drug delivery, particularly for targeted therapeutic applications. These sutures, either coated or infused with drugs, offer a means to administer medications directly to the intended site, thereby minimizing systemic exposure and related side effects. However, the restricted size and diameter of traditional sutures present considerable obstacles in terms of drug loading capacity. This constraint limits their use for extended or higher-dose drug administration. It will be valuable contribution to the art where capacity of suture to release the drug has been substantially increased.

Minimally invasive surgical (MIS) procedures are gaining popularity due to their benefits in shortening patient recovery times, reducing surgical trauma, and lowering complication risks. These techniques employ compact devices to insert therapeutic implants into the body. To accommodate the spatial limitations of MIS devices, drug delivery systems must be able to compress for insertion and then expand to their functional form once deployed. This requirement poses unique engineering and material challenges, especially for microsphere-based systems. Newer methods and devices that can be used by MIS systems can be a valuable contribution to the art.

Surgical and non-surgical procedures often require localized drug delivery systems to enhance therapeutic outcomes, manage post-operative complications, and reduce adverse effects associated with systemic drug administration. Despite advancements in drug delivery technologies, achieving controlled, localized, and sustained release of therapeutic agents at target sites remains a challenge. There is a need for versatile, modular drug delivery system designed for use in a variety of medical applications.

This invention represents a significant advancement in localized drug delivery, offering a multifunctional, customizable, and easy-to-use solution for diverse clinical applications.

The following patents are hereby incorporated herein by reference for all purposes. In case of conflict, the current specification is controlling. U.S. Pat. Nos. 10,624,865, 9,789,073, 9,498,557, 9,072,678, 8,506,856, 8,067,031, 7,919,112, 7,790,141, 7,740,877, 7,009,034, 6,887,974, 6,566,406, 6,387,977, 6,201,065, 6,004,573, 5,874,500, 5,741,323, 5,529,914, 5,410,016, 6,534,591, 7,740,877, 8,067,031, 9,023,379, 5,573,934 and US Patent Applications US 2024/0252452, US 2022/0118416, US 2019/0046479, US 2016/0166504, US 2015/0060699, US 2014/0256617, US 2008/0058787, US 2005/0069572.

The present invention addresses the foregoing need for better compositions and methods for sustained delivery of drugs and other applications. Accordingly, there is a need for such compositions, methods and devices as summarized herein in some detail.

### SUMMARY OF THE INVENTION

An object of the present invention is to provide an implantable surgical array, comprising a plurality of array elements selected from the group consisting of particles, microparticles, and combinations thereof, wherein each array element is interconnected by a flexible thread, and the thread length between adjacent array elements is substantially the same.

An object of the present invention is to provide an implantable surgical array, comprising a plurality of array elements selected from the group consisting of particles, microparticles, and combinations thereof, wherein each array element is interconnected by a flexible thread, and at least two adjacent array elements are separated by a thread length that is at least 10 percent longer or shorter than the thread length between other adjacent array elements in the array.

An object of the present invention is to provide an implantable surgical array, comprising a plurality of array elements selected from the group consisting of particles, microparticles, and combinations thereof, wherein each array element is interconnected by a flexible thread, and the array elements are locked mechanically or embedded in the flexible thread to prevent their migration along the thread.

An object of the present invention is to provide an implantable surgical array, comprising a plurality of array elements selected from the group consisting of particles, microparticles, and combinations thereof, wherein each array element is interconnected by a flexible thread, and at least one of the array elements is porous.

An object of the present invention is to provide an implantable surgical array, comprising a plurality of array elements selected from the group consisting of particles, microparticles, and combinations thereof, wherein each array element is interconnected by a flexible thread, and the device includes at least one terminal end with a thread length of 2 mm or greater.

An object of the present invention is to provide an implantable surgical array, comprising a plurality of array elements selected from the group consisting of particles, microparticles, and combinations thereof, wherein each array element is interconnected by a flexible thread, and the device is compressible by at least 10 percent of its original length and expandable back to its original shape.

An object of the present invention is to provide an implantable surgical array, comprising a plurality of array elements selected from the group consisting of particles, microparticles, and combinations thereof, wherein each array element is interconnected by a flexible thread, and at least one of the array elements comprises a crosslinked material.

An object of the present invention is to provide an implantable surgical array, comprising a plurality of array elements selected from the group consisting of particles, microparticles, and combinations thereof, wherein each array element is interconnected by a flexible thread, and at least one of the array elements comprises a live mammalian cell, live bacterial or active enzyme or catalyst.

An object of the present invention is to provide an implantable surgical array, comprising a plurality of array elements selected from the group consisting of particles, microparticles, and combinations thereof, wherein each array element is interconnected by a flexible thread, and the device is substantially dry, containing less than 5 percent water or solvent by weight, and absorbs at least 10 percent of its weight in water upon implantation.

An object of the present invention is to provide an implantable surgical array, comprising a plurality of array elements selected from the group consisting of particles, microparticles, and combinations thereof, wherein each array element is interconnected by a flexible thread, and the array elements are selected from hydrogels, organogels, or combinations thereof.

An object of the present invention is to provide an implantable surgical array, comprising a plurality of array elements selected from the group consisting of particles, microparticles, and combinations thereof, wherein each array element is interconnected by a flexible thread, and the device is configured to be cut into smaller sections without unraveling the array elements, allowing adjustment of the drug dose delivered.

An object of the present invention is to provide an implantable surgical array, comprising a plurality of array elements selected from the group consisting of particles, microparticles, and combinations thereof, wherein each array element is interconnected by a flexible thread, and the array elements comprise hydrogels, and drug-encapsulated particles are embedded within at least some of the hydrogels.

An object of the present invention is to provide an implantable surgical array, comprising a plurality of array elements selected from the group consisting of particles, microparticles, and combinations thereof, wherein each array element is interconnected by a flexible thread, and the array elements are in direct contact with one another.

An object of the present invention is to provide an implantable surgical array, comprising a plurality of array elements selected from the group consisting of particles, microparticles, and combinations thereof, wherein each array element is interconnected by a flexible thread, and the array elements are configured to encode unique information by varying at least one measurable property, selected from the group consisting of physical, chemical, or imaging properties, wherein the encoded information is represented as numeric or alphanumeric values based on differences in the measurable properties of the array elements, and wherein the encoded information is used for identifying the array during usage.

An object of the present invention is to provide a method for producing an implantable surgical array, comprising providing a plurality of particles or microparticles; providing a flexible thread; threading the particles onto the flexible thread, wherein the particles are spaced at predetermined intervals; and securing the particles in place on the thread by mechanically locking, embedding, or knotting to prevent migration.

An object of the present invention is to provide a method for producing an implantable surgical array, comprising preparing a photopolymerizable precursor solution comprising a polymerizable monomer and a photoinitiator; exposing a flexible thread to the precursor solution, embedding the thread within the precursor; selectively exposing the precursor to a light source through a patterned photomask, forming crosslinked particles along the thread; and removing unpolymerized precursor to create the array.

An object of the present invention is to provide a method for producing an implantable surgical array, comprising providing a mold with cavities configured to form particles; positioning a flexible thread within the mold cavities; adding a polymerizable composition to the cavities such that the thread is at least partially embedded in the composition; curing or crosslinking the polymerizable composition to form particles attached to the thread; and removing the cured device array from the mold.

An object of the present invention is to provide a method for producing an implantable surgical array, comprising depositing a precursor solution at specific junctions of a pre-formed thread mesh or grid; polymerizing the precursor solution at the junctions to form crosslinked array elements; and forming an array wherein the array elements are interconnected by the thread mesh.

An object of the present invention is to provide a method for producing an implantable surgical array, comprising preparing a plurality of particles or microparticles made from hydrogels or organogels; loading the particles with one or more bioactive agents; embedding the particles in a flexible thread by threading or bonding; and forming an array configured for sustained drug delivery.

An object of the present invention is to provide a method for producing an implantable surgical array, comprising providing a perforated film or textile material; applying a precursor solution at specific locations on the perforated film; polymerizing or curing the precursor solution to form array elements attached to the film; and cutting the film into desired shapes while retaining the array structure.

An object of the present invention is to provide a method for producing an implantable surgical array, comprising depositing a first droplet of a precursor solution onto a flexible thread; depositing a subsequent droplet of the precursor solution on the thread such that a minimum distance of 0.001 mm or more is maintained between the first droplet and the subsequent droplet; ensuring that the thread is partially or completely embedded within each droplet of the precursor solution; and initiating crosslinking of the precursor solution to form an array of interconnected elements along the thread.

An object of the present invention is to provide a method for producing an implantable surgical array, comprising providing a plurality of porous microparticles, each having a porosity of 10 percent or more; providing a flexible thread; threading the porous microparticles onto the flexible thread to form an array, wherein the microparticles are spaced at predetermined intervals or arranged in direct contact; and securing the microparticles in place on the thread by mechanically locking, embedding, or knotting to prevent migration along the thread.

An object of the present invention is to provide a method for producing an implantable surgical array, comprising providing a plurality of porous microparticles, each having a porosity of 10 percent or more; coating or embedding the porous microparticles with one or more drugs; providing a flexible thread; threading the drug-coated or drug-embedded porous microparticles onto the flexible thread to form an array, wherein the microparticles are spaced at predetermined intervals or arranged in direct contact; and securing the microparticles in place on the thread by mechanically locking, embedding, or knotting to prevent migration along the thread.

An object of the present invention is to provide a method for producing a drug-loaded implantable surgical array, comprising providing an array comprising a plurality of particles or microparticles interconnected by a flexible thread; incubating the entire array in an aqueous or organic solvent-based drug solution for a time sufficient to allow the drug to infuse into or coat the particles and the thread; removing the solvent or water from the array to produce a drug-loaded array, wherein the drug is embedded in or coated on the particles and the thread; and ensuring that the solvent used during incubation does not adversely affect the physical or chemical properties of the particles or the thread.

An object of the present invention is to provide an implantable surgical array, comprising a plurality of microparticles interconnected by a flexible thread; wherein the microparticles contain a drug embedded within or coated onto their structure; and wherein the flexible thread is free of any drug.

In one embodiment, the present invention provides an implantable array device comprising a plurality of elements encoded with unique numeric or alphanumeric identifiers based on their physical or chemical properties. These identifiers are generated through imaging techniques, such as X-ray or ultrasonic imaging, enabling the array to be used as a surgical or biopsy marker. The device is configured to be identifiable by its imaging signatures, allowing precise localization and identification post-implantation.

In another embodiment, the present invention provides a drug-loaded array device comprising a plurality of interconnected elements. Each element contains or is coated with therapeutic agents, and the device is configured to be sutured at a wound site. This allows for localized drug delivery, with adjustable dosing facilitated by cutting or extending the array. The device prevents infections, minimizes scar formation, and provides pain relief, enhancing wound healing outcomes.

In one embodiment, the present invention provides an implantable array device configured to be spirally wound around vascular grafts or junction of graft/artery interface. The device comprises a plurality of interconnected elements loaded with anti-restenosis agents, and a flexible thread component that provides anti-kinking properties. This configuration enhances the mechanical and therapeutic performance of the graft by ensuring targeted drug delivery and structural integrity during and after surgery.

In another embodiment, the present invention provides an implantable array device comprising a plurality of interconnected elements configured to deliver localized therapeutic agents to the gum and tooth interface. The device can be positioned in interdental spaces or along the gum line, where it releases therapeutic agents such as antibiotics, anti-inflammatory drugs, or germicides. This ensures sustained treatment of bacterial infections, inflammation, and gum diseases, while promoting improved oral hygiene.

In one embodiment, the present invention provides an implantable array device comprising a plurality of interconnected elements made of biodegradable materials. The device is configured to act as a physical barrier to prevent tissue adhesions post-surgery. Optionally, the device can be loaded with therapeutic agents such as anti-inflammatory or anti-fibrotic drugs to actively inhibit adhesion formation. The biodegradable nature of the device ensures its natural dissolution after serving its purpose.

In another embodiment, the present invention provides an array device comprising a plurality of hydrogel-based elements encapsulating active enzymes, live bacterial cells, or mammalian cells. The elements are configured to sustain the stability and activity of the encapsulated materials for industrial chemical production or biologic drug manufacturing. The array allows customization of porosity, composition, and arrangement to suit specific processes, enabling efficient and scalable production.

Yet in another embodiment, the present invention provides an implantable array device comprising: a plurality of array elements configured to be interconnected by at least one flexible thread, wherein the array elements are composed of biocompatible materials; at least one visual or tactile indicator integrated into the device, wherein the visual indicator provides user-perceivable guidance for specific actions, such as cutting, looping, positioning, or configuring the array; wherein the visual indicator is detectable by an unaided human eye or human touch and is configured to facilitate the customization, handling, or placement of the array device during surgical procedures or therapeutic applications; wherein the visual or tactile indicator is tailored to correspond to at least one of the following parameters: a predefined drug dosage, a specific point for structural modification, or a target positioning requirement within a biological environment

In one embodiment, the present invention provides a hydrogel-based array device comprising a plurality of elements functionalized with antibodies or analytical agents. The elements are configured to perform multiplex detection and measurement of biomarkers in biological samples. This capability allows for diagnostics such as disease detection, patient outcome monitoring, or treatment response evaluation with enhanced sensitivity and specificity.

In another embodiment, the present invention provides an implantable array device comprising a plurality of hollow or solid biodegradable metal beads interconnected by a flexible thread. The device is configured to be deployed as a temporary radiation shield, capable of filling large spaces uniformly. Optionally, the beads can be filled with a radio-opaque composition to enable real-time monitoring of coverage and biodegradability during and after deployment.

Yet another embodiment teaches a method for producing an array device. The method comprises: freezing liquid precursor droplets in a cryogenic bath selected from the group consisting of liquid nitrogen, liquid helium, solid carbon dioxide, or aqueous salt solutions; arranging the frozen droplets on a thread in a desired sequence and spacing, with or without the use of a mold; warming the frozen droplets to room temperature to form a liquid state, wherein the liquid compositions partially or substantially embed into the thread; and crosslinking and/or lyophilizing the liquid compositions to secure them to the thread, thereby forming a stable array whose elements are partially or substantially embedded in the array thread.

Another embodiment discloses a method for producing an array device. The method comprises: providing a low-melting polymer with a melting point below 70° C., preferably a biodegradable polymer; using a melt-dispensing device to form droplets of the polymer in a desired size; depositing the droplets onto a thread, with or without the use of a mold cavity, in a selected arrangement and spacing; allowing the melted polymer to partially, substantially, or completely embed into the thread; and cooling the polymer to solidify and secure it to the thread, thereby forming a stable array.

### BRIEF DESCRIPTION OF THE SEVERAL VIEWS OF THE DRAWINGS

The above-mentioned and other features and advantages of this present disclosure, and the manner of attaining them, will become more apparent and the present disclosure will be better understood by reference to the following description of embodiments of the present disclosure taken in conjunction with the accompanying drawings, wherein:

FIGS. 1A to 1L show a partial schematic representation of various interconnected particle arrays in either necklace-like or sheet-like configurations, according to one or more embodiments.

FIGS. 2A to 2J show a partial schematic representation of various configurations of necklace-like devices or interconnected particle array devices made using suture or thread and micro or macro particles, according to one or more embodiments.

FIGS. 3A to 3D demonstrate various methods for creating interconnected particle-based array devices, showcasing a range of techniques and material manipulations in accordance with one or more embodiments.

FIGS. 4A to 4E outline methods for creating drug delivery devices using biodegradable threads or films, each method incorporating various drug-loaded compositions, demonstrated using one or more embodiments.

FIGS. 5A to 5D2 illustrate several array devices made by threading a suture through the array elements, which are capable of existing in both expandable and compressed forms, according to one or more embodiments.

FIGS. 6A to 6C illustrate photolithographic methods to create array devices, detailing the use of photosensitive precursors and controlled exposure techniques in accordance with one or more embodiments.

FIGS. 7A to 7H display a variety of array configurations with diverse shapes and sizes, each designed to cater to specific functional and therapeutic needs in accordance with one or more embodiments.

FIGS. 8A to 8I showcase several array configurations with various shapes, sizes, and distinct features, each designed to meet specific therapeutic and operational needs in accordance with one or more embodiments.

FIGS. 9A to 9D highlight various methods of encoding unique information within arrays by leveraging the sequence of elements and their physical properties to generate numeric or alphanumeric codes for identification, according to one or more embodiments.

FIGS. 10A and 10B illustrate an ophthalmic device, such as a contact lens embedded with an array device, and a minimally invasive surgical (MIS) device used for deploying and/or retrieving the array device.

### DETAILED DESCRIPTION OF THE INVENTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting. Exemplary embodiments of the present invention are directed towards compositions, methods and devices for facilitating local and sustained drug delivery, other medical and non-medical applications.

It is advantageous to define several terms, phrases and acronyms before describing the invention in detail. It should be appreciated that the following terms are used throughout this application. Where the definition of terms departs from the commonly used meaning of the term, the applicant intends to utilize the definitions provided below, unless specifically indicated. The following definitions are provided to illustrate the terminology used in the present invention. Unless defined otherwise, all technical and scientific terms used herein have the same meaning as is commonly understood by one who is skilled in the art. All scientific literature and patent citations in this invention are incorporated herein for reference use only.

“Crosslinked material” is meant to denote the formation of intermolecular or intramolecular covalent bonds in the precursor or macromolecule or polymer. The crosslinked material may be lightly or highly swollen in organic solvents or aqueous solutions without dissolution.

A “crosslinking agent” is defined as a compound capable of forming cross linkages. For example, glutaraldehyde is generally known in the art as a crosslinking agent for albumin or collagen.

“In situ” is meant to denote a local site, especially within or in contact with living organisms, tissue, skin, organs, or the body. In some instances, the term in situ is also used to mark a local site within the microparticle or microimplant material body.

Bioactive” or “drug” as used herein, refers to one or all of the activities of a compound that show pharmacological or biological activity in human or animal body. Such biological activity is preferred to have therapeutic effect. The bioactive compounds that can be used include, but not limited to: antiviral agents; antiinfectives such as antibiotics; antipruritics; antipsychotics; cell cycle inhibitors, anticancer agents, antiparkinsonism drugs, antirestenosis agents, antiinflammatory agents; antiasthmatic agents; antihelmintics; immunosuppressives; glucagon-like peptide 1 (GLP-1); muscle relaxants; antidiuretic agents; vasodilators; nitric oxide, nitric oxide releasing compounds, beta-blockers; hormones; antidepressants; decongestants; calcium channel blockers; growth factors, wound healing agents, analgesics and analgesic combinations; local anesthetics agents, antihistamines; sedatives; angiogenesis promoting agents; angiogenesis inhibiting agents; tranquilizers, synthetic peptides such as RGD or IGD peptides, and the like.

The specific drugs may include but not limited to: tramadol. aproclonidine, brimonidine, ketocaonazole, ofloxacin, ketorolac tromethamine, pyrimethamine, prednisolone sodium phosphate, tetracaine HCl, dexamethasone, timolol, tobramycin, rimexolone, sulfadiazine, bromocriptine, flucytosine, cyclopentolate HCl, ganciclovir sodium, epinastine HCl, physostigmine, echothiophate, carbonic anhydrase inhibitors, fluocinolone acetonide, valganciclovir HCl, antazoline phosphate, medrysone, acetaminophen and codeine, levobunolol, vitamins A, Vitamin E, ceftriaxone, gentamicin, ephedrine hydrochloride, rose bengal, scopolamine HBr, suprofen, polymyxin B, norfloxacin, cephalexin, olopatadine HCl, antioxidants, azathioprine, lutein, diclofenac sodium, indocyanine green, doxycycline, carteolol, fluorescein, latanoprost, ibuprofen, acetaminophen, proparacaine HCl, uravoprost, sodium sulfacetamide, unoprostone cidofovir, dipivefrin, taurine, levocabastine HCl, fomivirsen sodium, homatropine, famciclovir, atropine sulfate, naphazoline hydrochloride, vancomycin, flurbiprofen sodium, bimatoprost, cromolyn sodium, fluconazole, emadastine difumerate, tropicamide, dexamethasone sodium phosphate, dorzolamide, prednisolone acetate, fluoromethalone acetate, sissamine green, ofloxacin, levofloxacin, ciprofloxacin, proparacaine HCl and fluorescein sodium, brinzolamide, phenylephrine HCl, tetrahydrozoline hydrochloride, lodoxamide tromethamine, sulfisoxazole diolamine, fluoromethalone, trifluridine, ketotifen fumerate, gatifloxacin, loteprednol etabonate, foscarnet sodium, phenylephrine hydrochloride, ketorolac, erythromycin, amikacin, cyclosporine, acyclovir, dicloxacillin, itraconazole, zeaxanthin, azelastine HCl, betaxolol, nedocromil sodium, amphotericin B, methazolamide; prostoglandins, prostamides amoxicillin/clavulante, rapamycin methotrexate, acetaminophen, hydrocodone, permirolast potassium, azithromycin, pheniramine maleate, benoxinate and fluorescein sodium, moxifloxacin, benoxnate, fluorexon disodium, sulfisoxazolone diolamine, epinephrines, acetazolamide, nutraceuticals, cefixime, glutathione, oxymetazoline, fluorexon, carbachol, pilocarpine, cholinesterase inhibitors, metipranolol, sodium sulfacetamide and tetracycline. Cellular elements, which can be used for therapeutic use, include, but are not limited to mammalian cells including stem cells; cellular components or fragments, enzymes, DNA, RNA, and genes may also be included as bioactive components or drugs. An extensive list of bioactive compounds or drugs that may be used can be found in U.S. Pat. No. 8,067,031 cited herein for reference only

The terms “Biodegradable”, “Bioerodible” and “Bioabsorbable” have the same meaning unless specified. The terms are meant to denote a material or substance, that will degrade in a biological environment such as the human body by either a biologically assisted mechanism, such as an enzyme catalyzed reaction or by a chemical mechanism which can occur in a biological medium, such as hydrolysis or by a dissolution mechanism in which the substance dissolves and is removed safely without any degradation.

“Biostable” is meant to denote the high chemical stability of a compound in an aqueous environment, which is similar to the environment found in the human body such as phosphate buffered saline (pH 7.4).

The term “biodegradable polymers” may include polymers or macromolecules which degrade/dissolve safely in the biological environment such as the human body. The term applies to polymers that are hydrophobic or hydrophilic. The term is applicable to polymers that are cross linked or not-crosslinked. The crosslinking may be done via condensation polymerization or via free radical polymerization or via ionic bonding. The biodegradable polymers may be random or block or graft copolymers. The biodegradable polymers may be linear, graft, dendramer or branched. The hydrophobic biodegradable polymers include, but are not limited to, polymers, dendrimers, copolymers or oligomers of glycolide, dl-lactide, d-lactide, 1-lactide, caprolactone, dioxanone and trimethylene carbonate; degradable polyurethanes; degradable polyurethanes made by block copolymers of degradable polylactone such as polycaprolactone and polycarbonate such as poly(hexamethylene carbonate); tyrosine-derived polycarbonates, tyrosine-derived polyacrylates, polyamides; polyesters; polypeptides; polyhydroxy acids; polylactic acid; polyglycolic acid; polyanhydrides; and polylactones. Biodegradable polymers also include polyhydroxyalkanoates which are polyesters produced by microorganisms including and not limited to poly(3-hydroxybutyrate), 3-hydroxyvalerate, 4-hydroxybutyrate, 3-hydroxyhexanoate, 3-hydroxyoctanoate. The term applies to hydrophilic polymers, which include, but are not limited to, polyethylene glycol-polyhydroxy acid or polyethylene glycol-polylacone copolymers (PEG-PL copolymers); polyvinyl alcohol-co-polylactide copolymers; and derivatives of cellulose; collagen or modified collagen derivatives; gelatin; albumin or cross linked albumin; fibrinogen; keratin; starch; hyaluronic acid and dextran.

The term “biostable polymers” include but are not limited to aliphatic and aromatic polyurethanes; polycarbonate polyurethane; polyether polyurethane; silicone polyurethane block copolymers; silicone rubbers; polydimethylsiloxane and its copolymers; polytetrafluoroethylene and other fluorinated polymers; expanded polytetrafluoroethylene; polyethylene; polyesters, polyethylene terephthalate, polyimides, polypropylene; polyamide; polyamide block copolymers and the like.

The term “non-crosslinked polymer/s” refers to any polymers or macromolecules that are not crosslinked and do not have the capability to form a covalent bond with precursor components under effective polymerization and crosslinking conditions. Generally, non-crosslinked polymers are linear, branched or dendrimer like organic solvent or water soluble polymers. Polylactide or polycaprolactone is a typical example of a “non-crosslinked polymer” linear polymer that is soluble in many organic solvents and is biodegradable. Preferred non-crosslinked polymers are biodegradable and end capped.

“Sustained release” or “controlled drug delivery” or “long term release” are phrases used interchangeably herein, to mean longer than the expected delivery of a bioactive compound from the inventive composition. Typically, delivery will be at least for one hour or more, two to six hours or more, and may extend to one day, a few days, weeks, months to a few years.

A “hydrogel” as used herein, refers to a semisolid composition constituting a substantial amount of water, and in which polymers, macromolecules or non-polymeric materials or mixtures thereof are dissolved or dispersed. The polymers may be physically or chemically crosslinked or not crosslinked.

An “organic solvent gel” or “Organic Solvent Gel (OSG)” refers to a semisolid composition constituting a substantial amount of organic solvent, and in which polymers, macromolecules or non-polymeric materials or mixtures thereof are dissolved or dispersed. The gel compositions may be physically or chemically crosslinked or not crosslinked.

Polyethylene glycol (PEG) or polyethylene oxide (PEO) refers to the polymer made by polymerization of ethylene oxide.

Polypropylene glycol (PPG) or polypropylene oxide (PPO) refers to the polymer made by the polymerization of propylene oxide.

Polymeric nomenclature used in this patent application such as poly(ethylene glycol) or polyethyleneglycol or polyethylene glycol refers to the same polymer unless otherwise stated clearly. This is also true for all other polymers referred to in this patent application.

The term “micron” means a length of 1/1000000 of a meter; one μl means 1/1000000 of a liter, 1 nanoliter means 1/1000000000 of a liter, and one picoliter means 1/1000000000000 of a liter and one femtoliters means 1/1000000000000000 of a liter.

The term “macromonomer” or “macromer” refers to oligomeric or polymeric materials capable of undergoing free radical polymerization or addition polymerization.

The term “hydrophobic” is defined as a property of materials or polymers or macromolecules having a low degree of water absorption or attraction.

The terms “coloring compositions” include any coloring composition or chemical that is suitable for human or animal implantation and are preferably approved by FDA for use in implantable medical devices or in pharmaceutical preparations, especially injectable pharmaceutical preparations. The compounds include but are not limited to: Methylene blue; Indocyanine green, Eosin Y; Ethyl Eosin, Fluorescein sodium; Chromium-cobalt-aluminum oxide; Ferric ammonium citrate; Pyrogallol; Logwood extract; 1,4-Bis[(2-hydroxy-ethyl)amino]-9,10-anthracenedione bis(2-propenoic)ester copolymers(3; 1,4-Bis [(2-methylphenyl)amino]-9,10-anthracenedione; 1,4-Bis[4-(2-methacryloxyethyl) phenylamino] anthraquinone copolymers; Carbazole violet; Chlorophyllin-copper complex, oil soluble; Chromium-cobalt-aluminum oxide; Chromium oxide greens; C.I. Vat Orange 1; 2-[[2,5-Diethoxy-4-[(4-methylphenyl)thiol]phenyl]azo]-1,3,5-benzenetriol; 16,23-Dihydrodinaphtho [2,3-a:2′,3′-i]naphth [2′,3′:6,7]indolo [2,3-c]carbazole-5,10,15,17,22,24-hexone; N,N′-(9,10-Dihydro-9,10-dioxo-1,5-anthracenediyl) bis benzamide; 7,16-Dichloro-6,15-dihydro-5,9,14,18-anthrazinetetrone; 16,17-Dimethoxydinaphtho (1,2,3-cd:3′,2′,1′-1m) perylene-5,10-dione; Poly(hydroxyethyl methacrylate)-dye copolymers: one or more of Reactive Black 5; Reactive Blue 21; Reactive Orange 78; Reactive Yellow 15; Reactive Blue No. 19; Reactive Blue No. 4; C.I. Reactive Red 11; C.I. Reactive Yellow 86; C.I. Reactive Blue 163; C.I. Reactive Red 180; 4-[(2,4-dimethylphenyl)azo]-2,4-dihydro-5-methyl-2-phenyl-3H-pyrazol-3-one; 6-Ethoxy-2-(6-ethoxy-3-oxobenzo[b]thien-2(3H)-ylidene) benzo[b]thiophen-3(2H)-one; Phthalocyanine green; Iron oxides; Titanium dioxide; Vinyl alcohol/methyl methacrylate-dye reaction products; one or more of: (1) C.I. Reactive Red 180; C.I. Reactive Black 5; C.I. Reactive Orange 78; C.I. Reactive Yellow 15; C.I. Reactive Blue No. 19; C.I. Reactive Blue 21; Mica-based pearlescent pigments; Disodium 1-amino-4-[[4-[(2-bromo-1-oxoallyl)amino]-2-sulphonatophenyl]amino]-9,10-dihydro-9,10-dioxoanthracene-2-sulphonate (Reactive Blue 69); D&C Blue No. 9; D&C Green No. 5; [Phthalocyaninato(2-)] copper; FD&C Blue No. 2; D&C Blue No. 6; D&C Green No. 6; D&C Red No. 17; D&C Violet No. 2; D&C Yellow No. 10; and the like. Preferred colored compositions are biodegradable.

The term “minimally invasive surgery” or (MIS) used herein includes, but is not limited to, surgical techniques such as, by way of example, and not limitation, laparoscopy, thoracoscopy, arthroscopy, intraluminal endoscopy, endovascular techniques, catheter-based cardiac techniques (such as, by way of example, and not limitation, balloon angioplasty), and interventional radiology.

The terms “molecular mass” “molecular weight” and “molar mass” have often been used interchangeably and are generally referred to as mass of a given molecule measured in Daltons (Da) or atomic mass units. The molecular weight is also expressed as g/mole in some instances and is the same as Daltons.

“Polylactic acid” or “poly(lactic acid)” or “poly(lactide)” or PLA is a term used for a polymer which is made from lactide or lactic acid. Similarly, PGA is a term used for polyglycolic acid or poly glycolate. Some synthetic biodegradable polyester polymers are generally referred to as poly lactones or polyhydroxy acids. The terms “PLGA” and “PDLG” refer to the same polymer and is a copolymer of PLA and PGA.

The term “exposing” refers to soaking the desired material in a fluid comprising the treatment agent for a period sufficient to treat the desired material. The soaking may be performed by, but is not limited to, incubation, swirling, immersion, mixing, or vortexing.

The term “Precursor/s” refers to chemical/s that is transformed into another compound via a chemical reaction. It is generally referred to a compound that is converted into a physically or chemically crosslinked hydrogel or organic solvent gel via physical interactions or chemical reactions. More specifically, the term generally refers to monomer/s or macromolecules that are transferred into a molecular crosslinked network via free radical/addition polymerization reaction or condensation polymerization reaction. The term also applies to macromolecules such as gelatin or albumin that are crosslinked using a crosslinker such as glutaraldehyde to form a crosslinked polymer or hydrogel.

The term “polymerizable” denotes the characteristic of molecules that have the capacity to form additional covalent bonds resulting in interlinking or covalent bonding of monomers and/or polymers to form oligomers or polymers. For example, molecules that contain acrylate type bonds form polymers using a free radical polymerization mechanism under effective reaction conditions. Molecules that have functional groups capable of reacting with each other leading to oligomer or polymer formation using condensation polymerization under effective reaction conditions.

“Effective Polymerization” refers to the chemical reaction of polymerization that leads to successful polymerization of polymerizable monomers to form polymer, oligomer or crosslinked networks. Certain physical and chemical conditions must be provided for effective polymerization of monomers.

The term “water soluble” generally refers to the solubility of a compound in water wherein the compound has a solubility of greater than 1 g/100 g, preferably greater than 5 g/100 g in distilled water.

The term “water insoluble” generally refers to the solubility of a compound in water wherein the compound has a solubility of less than 5 g/100 g, preferably less than 1 g/100 g in distilled water.

The term “organic soluble” or “organic solvent soluble” generally refers to the solubility of a compound in an organic solvent wherein the compound has a solubility of greater than 1 g/100 g, preferably greater than 5 g/100 g in an organic solvent.

The terms “imaging agent(s)” or “visualization agent(s)” include any medical imaging agent that helps to visualize the human body/tissue using the naked human eye or using machine assisted viewing. The term generally applies to but not limited to: coloring compositions that introduce color to medical devices and drug delivery compositions, radio-opaque contrast agents that help to visualize organs/tissues using x-ray imaging techniques, NMR contrast agents that assist in MRI imaging techniques, an ultrasonic contrast agent that improve imaging using ultrasound techniques and the like.

The term “Porosity” is defined as the presence of pores, voids, cavities, grooves, pockets and indentations within a material or on its surface or both.

Terms such as “Bead/s”, “Disk/s”, and “Array Elements” when used in the context of interconnected arrays typically refer to the component of the array device that is embedded or threaded within the device.

The present invention is now described with reference to the drawings.

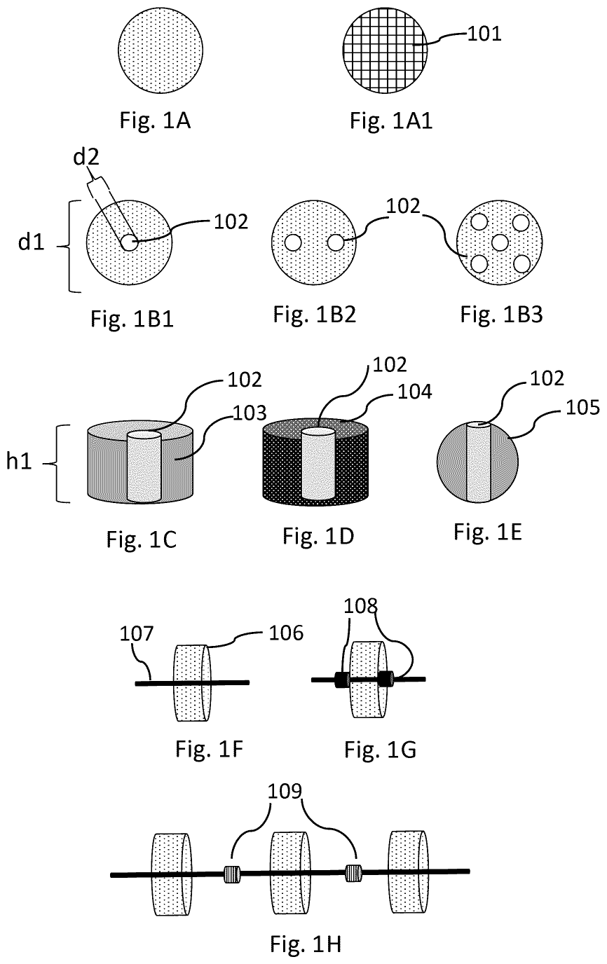

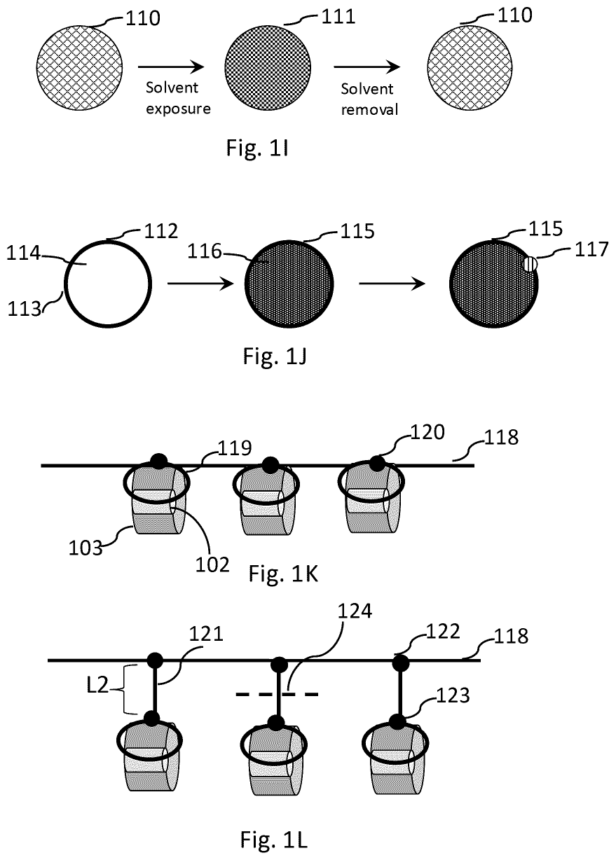

FIGS. 1A to 1J show a partial schematic representation of various interconnected particle arrays in either necklace-like or sheet-like configurations, as per one or more embodiments.

FIG. 1A depicts a disk-shaped cylindrical particle used as an array element in the array devices described in this invention, viewed from above, preferably biodegradable and capable of releasing a drug in a controlled manner over a period ranging from 1 hour to 3 years.

FIG. 1A1 is akin to FIG. 1A but is reinforced with either biodegradable or biostable mesh or fiber matrix (101) to augment its tear resistance.

FIG. 1B1 is similar to FIG. 1A but features a central hole (102), with d1 as the diameter of the particle and d2 as the diameter of the hole. FIGS. 1B2 and 1B3 are variations of FIG. 1B1 with 2 and 5 holes, respectively.

FIG. 1C offers a side view of FIG. 1B1, showcasing the central hole (102) along the axis of the cylindrical body (103) with height h1.

FIG. 1D is a modified version of FIG. 1C by either partially or completely coating/infusing the array element composition with drug/visualization agents (104).

FIG. 1E is akin to FIG. 1C but features a spherical shaped body (105) and a central hole (102).

FIG. 1F is an array device made by utilizing particles as shown in FIGS. 1A to 1E. A suture is threaded through the body of the particle depicted in FIG. 1A, with excess suture thread at either end (107), used for attachment or sewing to tissue or skin.

FIG. 1G resembles FIG. 1F but includes knots or barriers (108) on the suture to prevent the movement/migration of the particle body along the suture thread.

FIG. 1H is a device formed by combining or joining two or more devices/apparatuses as shown in FIG. 1F or 1G, with 109 indicating the attachment point such as a knot, adhesive joint, weld, or connecting joint on the suture.

FIG. 1I illustrates a process to soften a bead prior to threading. A hard plastic or crosslinked polymer bead (110), challenging to thread due to its hardness, is exposed to a biocompatible aqueous or organic solvent/solution for a sufficient duration to induce temporary softness, making it suitable for threading into an array. 111 shows the softened bead (110) after exposure to the solvent or solvent mixture, where the solvent is subsequently removed from the bead 111 to substantially restore its original size and shape.

FIG. 1J describes one of the processes for delivering a drug from a hollow bead or disk as an array element. A spherical hollow bead (112), with 113 as its outer casing of desired thickness and 114 as an empty space, is provided. The empty space (114) is filled with a drug delivery composition/visualization agent, either liquid or solid (116). 117 illustrates an opening/hole on bead 115, preferably a pinhole in the 113 casing wall, utilized for the controlled release of the drug or to infuse the drug delivery composition inside the space (114). Alternatively, the particle 115 may be an osmotic pump-based drug delivery device.

FIG. 1K shows a three particle array made from a particle with one hole (FIG. 1D) and an array thread (118). Array is made from array thread (118) that is looped through the 103 particle via 102 hole and the loop (119) is secured via a thread knot 120.

FIG. 1L is variation of array shown in FIG. 1K wherein an extra thread (121) with length L2 is used to connect the particle to the thread 118 via suture knots 122 and 123. 124 shows a cutting plane that can be used to cut or remove array element from the array without unraveling the other array elements. The cutting can be done anywhere along the 121 thread preferably closer to the array thread 118. The removal of array element enables to adjust drug dose if needed during implantation.

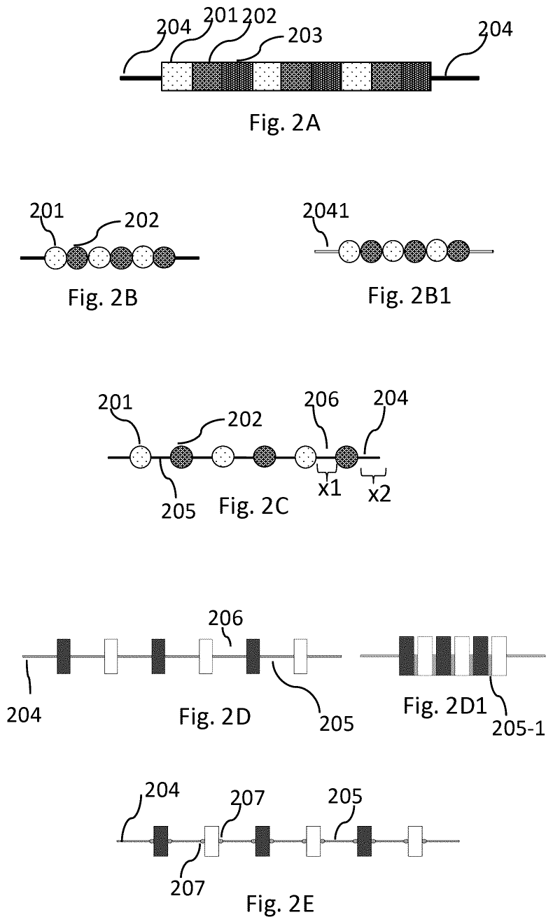

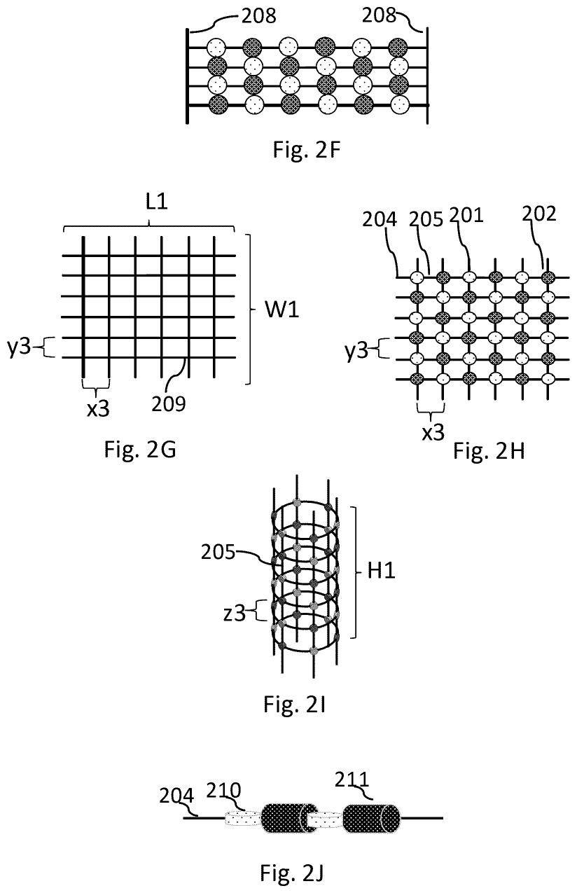

FIGS. 2A to 2J show a partial schematic representation of various configurations of necklace-like devices or interconnected particle array devices made using suture or thread and micro or macro particles, according to one or more embodiments.

FIG. 2A depicts an interconnected particle array device comprising cubical shaped beads or array elements threaded/embedded in a suture or thread. Elements 201, 202, and 203 schematically represent three different illustrative array element compositions, preferably drug delivery compositions within the array device. An excess thread on both ends of the device is shown as 204, which can be used for attaching/immobilizing the device to the body, skin, or tissue.

FIG. 2B is similar to FIG. 2A, where cubical shaped particles are replaced with two spherical/disk shaped particles, and their sequence is repeated in an alternate fashion three times. The particles in the array are in close contact with each other wherein the surface of array element or particle is in contact with its neighboring particle. FIG. 2B1 is similar to FIG. 2B, except the connecting fiber/thread/tube (2041) is hollow.

FIG. 2C is similar to FIG. 2B, except there is a desired amount of space between the array particles (206), and the particles are connected via thread (205) through the space 206. The length of thread between the particles (205) is shown as x1, and the length of thread at the terminal (204) end is shown as x2.

FIG. 2D is similar to FIG. 2C, wherein the thread is connected through the center of the disk-shaped particle (parallel/aligned to the cylindrical axis of disk), and 205 represents the thread between each particle. FIG. 2 shows a folded configuration of D where the space 206 is substantially minimized by folding the thread segments (205) to take a compact configuration (205-1), thus making the device D in a compact form which then can be delivered at a desirable location in the body using a minimally invasive surgical device.

FIG. 2E is similar to FIG. 2D, except a knot or barrier (207) is placed at both ends to prevent migration of particles along the thread.

FIG. 2F is a sheet-like structure formed by combining/joining several FIG. 2C like devices using 204 terminal thread ends and an extra thread (208) on both sides.

FIG. 2G shows a rectangular grid/matrix of suture/thread mesh with length L1 and width W1, wherein two fibers intersect in the matrix at 209. X3 and Y3 represent the length of suture between two 209 points along the length and width of the matrix, respectively.

FIG. 2H shows a sheet-like array device made using the fiber mesh shown in FIG. 2G, wherein array elements (201 and 202) are placed at the junction of two fibers intersection point (209).

FIG. 2I is a 3-dimensional cylindrical version of FIG. 2H wherein terminal ends of the device in FIG. 2H are joined to make a cylindrical shape with height H1, and particles in the device are arranged in a cylindrical format. The z3 shows the length of suture between two 209 points along the z direction.

FIG. 2J shows a schematic of an array containing a disk-like element (210) such as a biodegradable hydrogel that is separated by solid rigid cylindrical biodegradable plastic particles/tubes such as PLGA particles (211). 204 shows the terminal thread portion of the device. The rigid particle/tube 211 enables locking the position of 210 hydrogel elements in the array and making it migration-resistant.

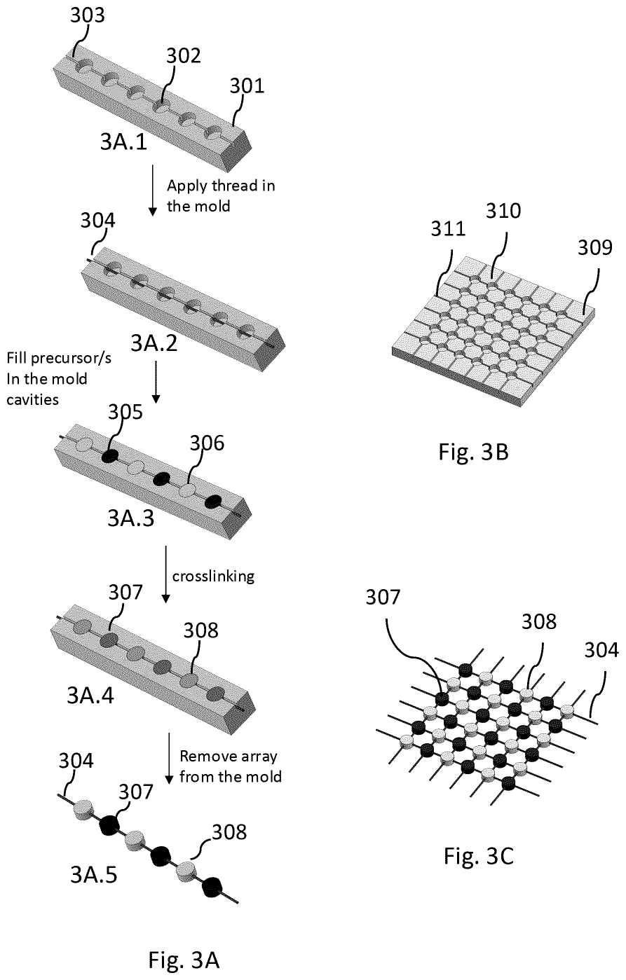

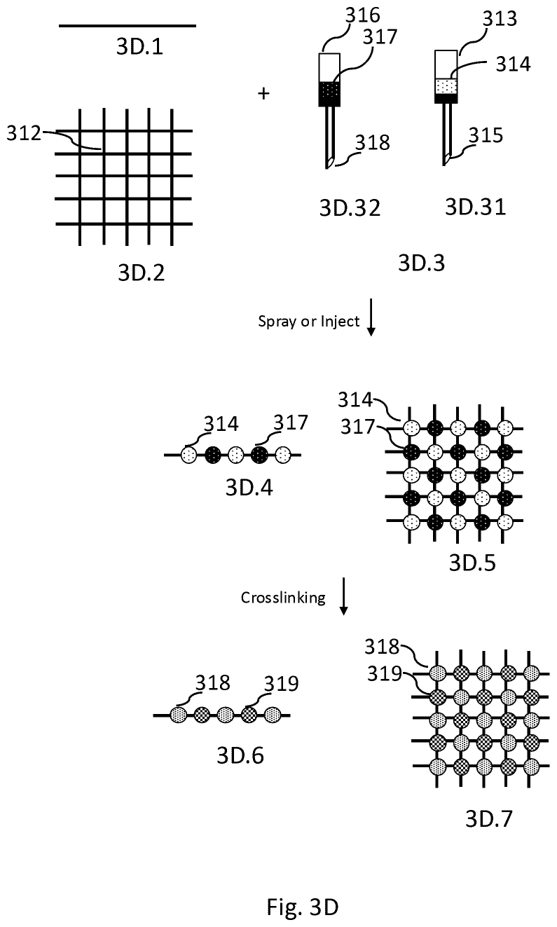

FIGS. 3A to 3E demonstrate various methods for creating interconnected particle-based array devices, showcasing a range of techniques and material manipulations in accordance with one or more embodiments.

FIG. 3A illustrates a flow diagram of a process to manufacture array devices using liquid compositions and/or precursors utilizing molds. Panel 3A.1 details a mold body (301) with multiple cylindrical cavities (302) and an optional line-shaped groove or cavity for placing a suture or thread (303). Panel 3A.2 shows a thread (304) positioned within the 303 mold cavity. Panel 3A.3 depicts the mold cavities (302) partially or completely filled with different liquid precursor compositions (305 and 306), with the thread (304) partially or completely immersed in these compositions. These precursor compositions are effectively polymerized by exposure to light or other crosslinking mechanisms (307 and 308), securing the thread (304) within the polymerized and crosslinked compositions, as shown in Panel 3A.4. The final polymerized beads/disks with embedded thread (array device) are then removed from the mold, wherein the crosslinked beads/disks (307 and 308) are embedded in the thread, as illustrated in Panel 3A.5.

FIG. 3B presents a mold for preparing a sheet/mesh-like array device using a modified version of the method described in FIG. 3A. The mold body (309) features multiple mold cavities (310) arranged in a matrix format (6 columns by 6 rows). Optionally, the mold has a groove to place a thread (311). Threads and precursor compositions are added to the cavities of the mold and then polymerized and crosslinked as described in FIG. 3A.

FIG. 3C displays a matrix-like array device removed from the FIG. 3C mold, showing a 6 by 6 array matrix wherein all beads are interconnected by suture thread and the crosslinked compositions (307 and 308) are located at the intersection of two thread junctions.

FIG. 3D shows a flow diagram for creating an array device using injectable or sprayable liquid precursor compositions. Panel 3D.1 displays a thread, and Panel 3D.2 shows a thread mesh with 25 thread junction points (312). Apparatuses in Panel 3D.3 possess the ability to form fine droplets or spray the desired amount of precursor solution. Panels 3D.31 and 3D.32 illustrate a print head or a syringe with needle (313 and 316), filled with precursor solutions (314 and 317) that can be injected or sprayed via nozzles (315 and 318). The solutions are deposited/sprayed along the thread in a desired sequence (Panel 3D.4) or at the junction point 312 on Panel 3D.2 mesh (Panel 3D.5). The deposited solution is then crosslinked to produce either a single thread array device (Panel 3D.6) or a matrix-type device (Panel 3D.7), wherein the crosslinked compositions (318 and 319) are attached/embedded in the thread or at the intersection of two threads.

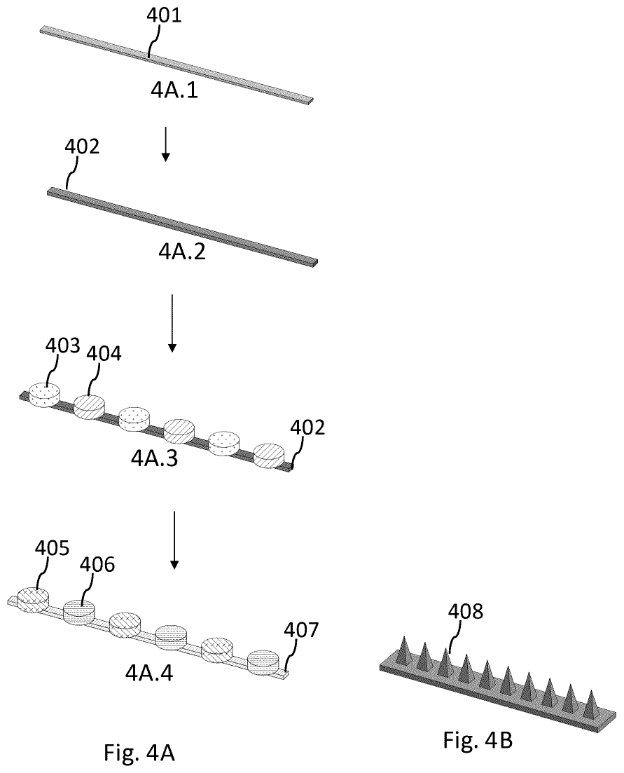

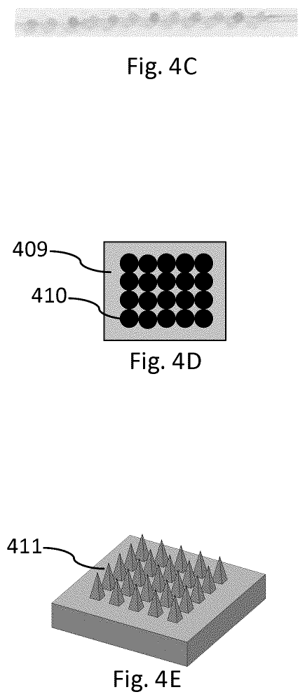

FIGS. 4A to 4E outline methods for creating drug delivery devices using biodegradable threads or films, each method incorporating various drug-loaded compositions, demonstrated using one or more embodiments.

FIG. 4A details a method for assembling a drug delivery device wherein several microparticles are connected to biodegradable threads/filaments or strips. Panel 4A.1 displays biodegradable strips or filaments, such as sections of sheep submucosa tissue (401), coated with a precursor composition like gelatin methacrylate solution (402) in water with a photoinitiator. The strip is subsequently cooled to 0 to −10 degrees Celsius to freeze the aqueous precursor composition. Separately, precursor compositions comprising gelatin methacrylate or PEG methacrylate with drug/visualization agents are filled in cylindrical mold cavities and frozen. The frozen precursor compositions (403 and 404) are then placed atop the coated strip 402 in a desired sequence, as depicted in Panel 4A.3. The entire assembly is then exposed to long UV light under frozen conditions to effectively polymerize and crosslink 403, 404, and the coating on the strip (402) to form crosslinked compositions (405 and 406) and a crosslinked coating composition (407), covalently linking the polymerized disks (405 and 406) to the tissue strip via the crosslinked coating 407, forming an array device (Panel 4A.4).

FIG. 4B shows an array device similar to that depicted in Panel 4A.4, where disks are replaced with microneedles.

FIG. 4C displays an image of the hydrogel array prepared according to one or more embodiments, wherein circular hydrogel disks are arrayed with desired thread length between the disks.

FIG. 4D illustrates a drug delivery device where biodegradable spheres or microspheres comprising drug/visualization agents (409) are attached/bonded to the biodegradable prosthetic tissue (410) or biodegradable polymer film/textile via crosslinked coating (407) to create a drug delivery patch.

FIG. 4E is similar to FIG. 4H, wherein microspheres are replaced with an array of microneedles (411).

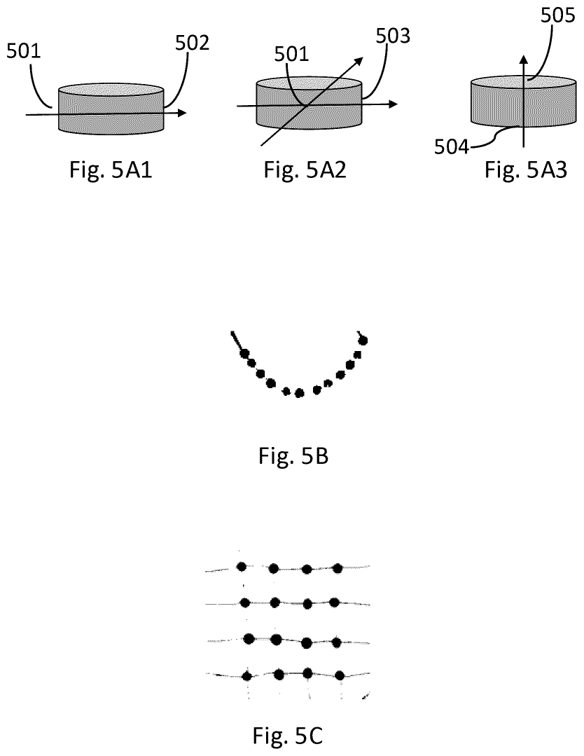

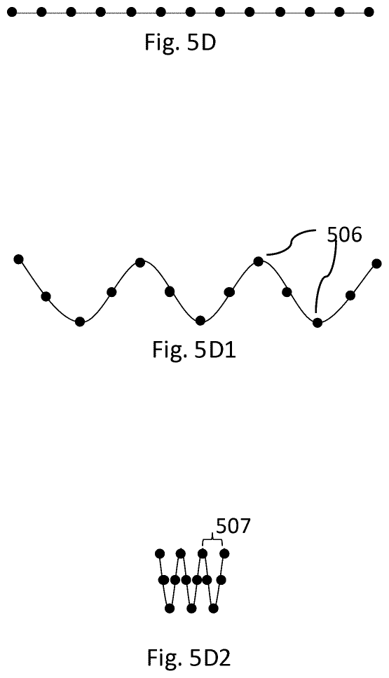

FIGS. 5A to 5D2 illustrate several array devices made by threading a suture through the array elements, which are capable of existing in both expandable and compressed forms, according to one or more embodiments.

FIG. 5A1 shows a disk-shaped cylindrical array element or controlled drug delivery implant that is used in making an array device via a threading method. Several disks are threaded to create an array device. A suture needle is threaded through the sidewall (via insertion point 501 and exit point 502, parallel to the circular plane and perpendicular to the axis of the cylindrical implant).

FIG. 5A2 is similar to FIG. 5A1, wherein two sutures or threads are inserted from two sites in the sidewall (503 and 501), with 503 at a 90-degree angle relative to 501.

FIG. 5A3 displays a disk-shaped cylindrical array element or controlled drug delivery implant used to make an array device using s suture threading method. A suture needle is threaded through the bottom of the cylinder (at the bottom circular face of the disk at the center point, 504). The needle exits at the top center point (505), with the thread running parallel to the disk axis. Several disks are threaded in a similar manner to form an array device.

FIG. 5B shows an image of a hydrogel array device made by threading a suture through several cylindrical hydrogel disks using a method as described in FIG. 5A1.

FIG. 5C depicts an image of a matrix-type array device prepared by threading from two sides (503 and 501), with the disk present at the intersection of two suture threads.

FIG. 5D shows a schematic of a linear array device wherein several array elements are interconnected by a thread. The device (FIG. 5D) is folded along the length at five folding points (506) to produce a compact, sine wave, or spiral-like shape array (FIG. 5D1). The device (Panel FIG. 5D1) is further compressed along the length to reduce its overall length and to produce a more compact form (FIG. 5D2). The pitch (507) of the device in FIG. 5D2 is less than the pitch of the device shown in FIG. 5D1. The number of folds and pitch length are varied to produce various compact and expanded forms of the array device (FIG. 5D).

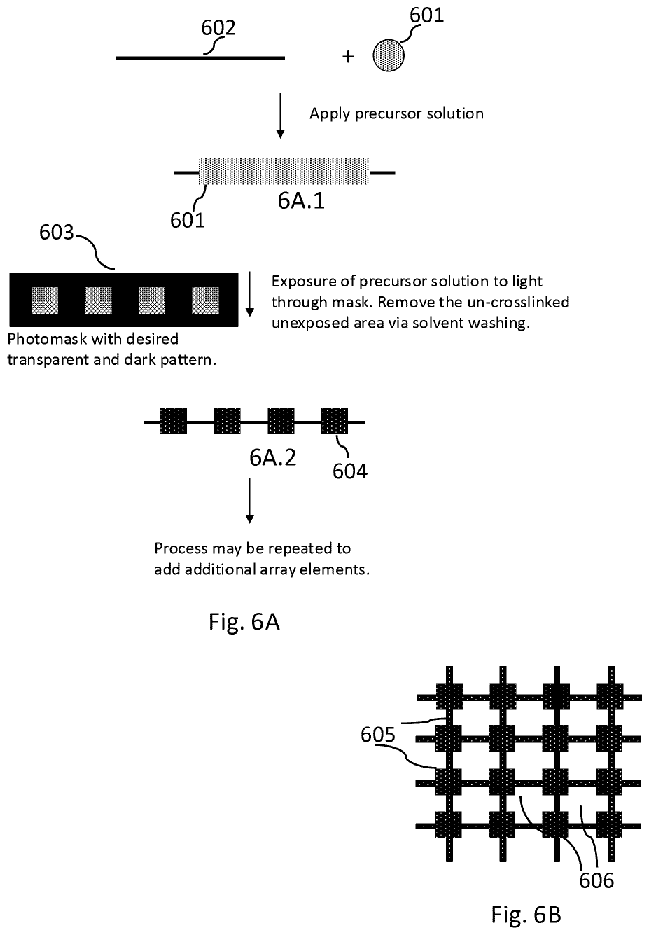

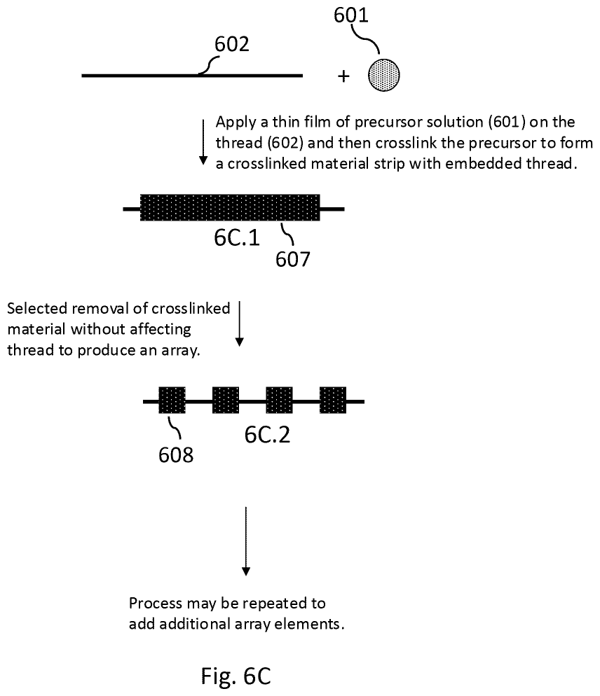

FIGS. 6A to 6C illustrate photolithographic methods to create array devices, detailing the use of photosensitive precursors and controlled exposure techniques in accordance with one or more embodiments.

FIG. 6A outlines a method for creating arrays from a precursor solution using a photolithography-like method involving a photosensitive precursor. A photopolymerizable precursor such as a gelatin methacrylate solution with a photoinitiator that polymerizes when exposed to 360 nm light (601) is applied over a thread (602) along with a photomask (603) that has a desired pattern of transparent and opaque areas. The precursor solution (601) is applied as a continuous layer along the thread, with the terminal portion of the thread remaining uncovered. Upon exposure to 360 nm light through the transparent portions of the mask (603), the precursor solution polymerizes and crosslinks only in the areas exposed to light, while the unpolymerized portion in the unexposed areas is removed by solvent washing or other methods, leaving behind an array with crosslinked precursor material (604) as shown in panel FIG. 6A2. This process can be repeated to add array elements using the same or different precursor compositions along the remaining portions of the thread.

FIG. 6B presents an array where the thread is replaced by a thin strip of array element material, produced using a modified method described in FIG. 6A. This method uses a different photomask wherein white portions (606) between the crosslinked material (605) are shielded from light exposure during the polymerization process. The unpolymerized portion is subsequently washed away to produce a matrix-like array structure without thread but with thin strip of element material as shown in FIG. 6B.

FIG. 6C demonstrates a method for creating an array device where specific portions of the crosslinked materials are selectively removed to form an array structure. A photopolymerizable precursor (601) and thread (602) are provided, with a thin film of precursor solution (601) applied on the thread (602) except at the terminal ends. The precursor is crosslinked (607) embedding the thread except at the terminal ends (Panel 6C.1). Parts of the crosslinked material (607) are then selectively removed using techniques such as laser ablation, mechanical cutting, or selective dissolution/degradation, without affecting the thread, thus creating an array-like structure with thread positioned between two array elements (608, shown in Panel 6C.2).

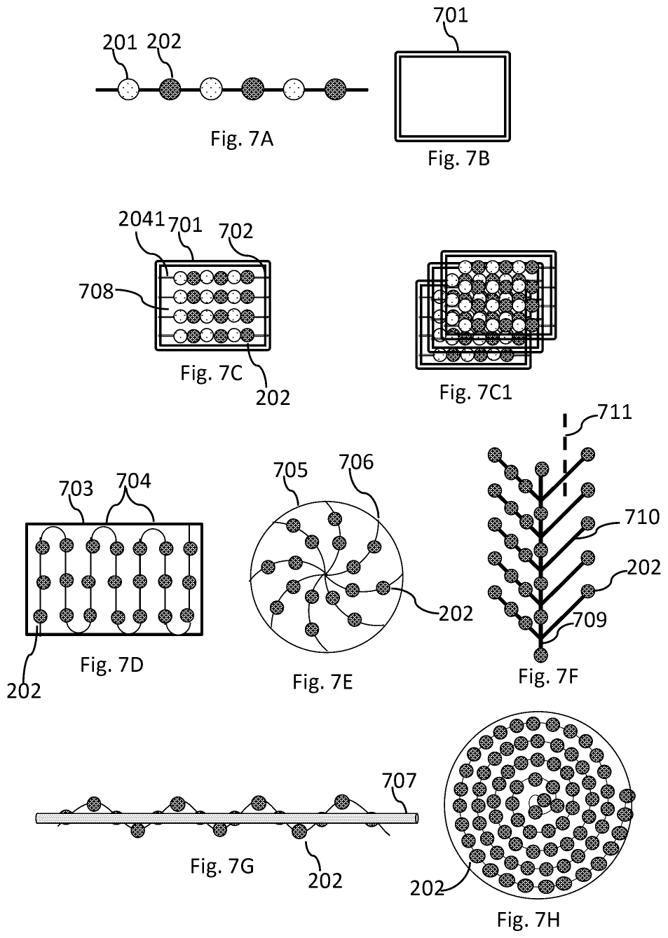

FIGS. 7A to 7H display a variety of array configurations with diverse shapes and sizes, each designed to cater to specific functional and therapeutic needs in accordance with one or more embodiments.

FIG. 7A shows a linear array with beads, which can be used in various therapeutic applications due to its simple yet versatile design.

FIG. 7B depicts a rectangular hollow frame (701), which serves as the structural backbone for more complex array configurations.

FIG. 7C and FIG. 7C1 illustrate several linear arrays with hollow tubing or thread (shown as in FIG. 2B1) that are attached to the frame 701. The connection at point 702 provides an open fluidic connection between the hollow frame and hollow thread/tube (2041), facilitating the transfer of gases, nutrients, or waste products if hydrogel beads (used in FIGS. 201 and 202) encapsulate live cells or active enzymes.

FIG. 7D highlights linear array elements looped around several times within a solid frame (703), with attachment points (704) used to increase the density of beads/elements per frame, optimizing the functional capacity of the array.

FIG. 7E shows a single point spiral shaped array in a circular frame (705) with 706 as thread attachment point to the frame.

FIG. 7F shows a branched array with 709 as a main or central array and with 5 branches (710) on either side of main branch 709. One of the branches shows 3 array elements (202) and other shows one terminal array element. The branches can be cut if desired along the cutting plane 711 without affecting other branches. This may be useful in adjusting a drug dose if desired.

FIG. 7G shows a linear array that is spirally placed/wound around tubular organ such as artery (708) or device such as vascular graft.

FIG. 7H shows a linear array device in a spiral configuration wherein all array elements are closely packed with each other.

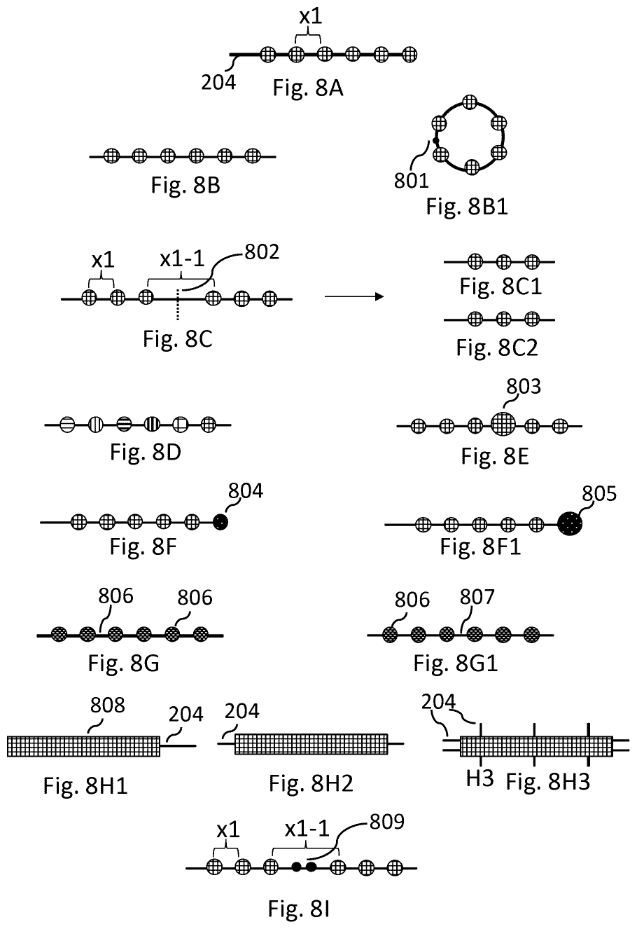

FIGS. 8A to 8I showcase several array configurations with various shapes, sizes, and distinct features, each designed to meet specific therapeutic and operational needs in accordance with one or more embodiments.

FIG. 8A illustrates an array device with spherical elements interconnected by a single thread, maintaining uniform spacing (x1) between elements and featuring one terminal attachment thread (204) at one end and no terminal thread at the other.

FIG. 8B depicts an array similar to that shown in FIG. 8A but with two terminal threads at both ends, enhancing structural stability and attachment options. FIG. 8B1 shows a similar design with both terminal threads tied together at a central attachment point (801) to form a circular or elliptical configuration, which may facilitate specific anatomical fits or therapeutic applications.

FIG. 8C presents an array similar to FIG. 8B but with varied thread lengths between the array elements, including at least two distinct lengths, X1 and X1−1, where X1−1 is approximately twice as long as X1; this array is sectioned along plane 802 to produce two separate arrays as shown in FIGS. 8C1 and 8C2, offering modular adaptability for customized therapeutic interventions.

FIG. 8D showcases an array with embedded drugs or visualization agents, represented by color gradients where lighter shades indicate lower drug concentrations and darker shades signify higher concentrations, enabling visual assessment of dosage and distribution within the array.

FIG. 8E features an array that includes at least one element (803) that has a larger size than the other elements in the array, designed to act as a central depot for controlled release or to house multiple therapeutic agents or other useful indicators.

FIG. 8F and FIG. 8F1 illustrate an array resembling the one in FIG. 8B but with at least one element (804) that exhibits enhanced water swelling capacities, preferably upon implantation in the body. FIG. 8F1 demonstrates the increase in size of element 804 upon implantation or in contact with water due to substantial water absorption (805), which could be advantageous for maintaining or expanding the device's position within a physiological site.

FIG. 8G and FIG. 8G1 highlight an array where drugs are loaded into both the thread (806) and the array elements for controlled drug delivery. FIG. 8G typically involves incubating the entire array in a drug solution and infusing/coating the drug on both the thread and the array elements.

FIG. 8G1 depicts a similar array where drugs are infused/coated only in the array elements (806) and not in the thread (807), typically achieved by embedding or mechanically threading the array elements.

FIG. 8H to FIG. 8H3 explore continuous element composition along the thread length, with FIG. 8H showing an array with continuous element composition and one terminal thread. FIG. 8H1 to 8H3 progressively introduce additional structural complexities, including multiple threads or a matrix of threads with multiple anchoring points (204) on the terminal or on the side, providing enhanced structural integrity and modular connectivity for complex therapeutic applications.

FIG. 8I is similar to FIG. 8B or 8C array but has one, two or more knots (809) on the suture that serve as visual or tactile indicators to indicate certain features of the array device.

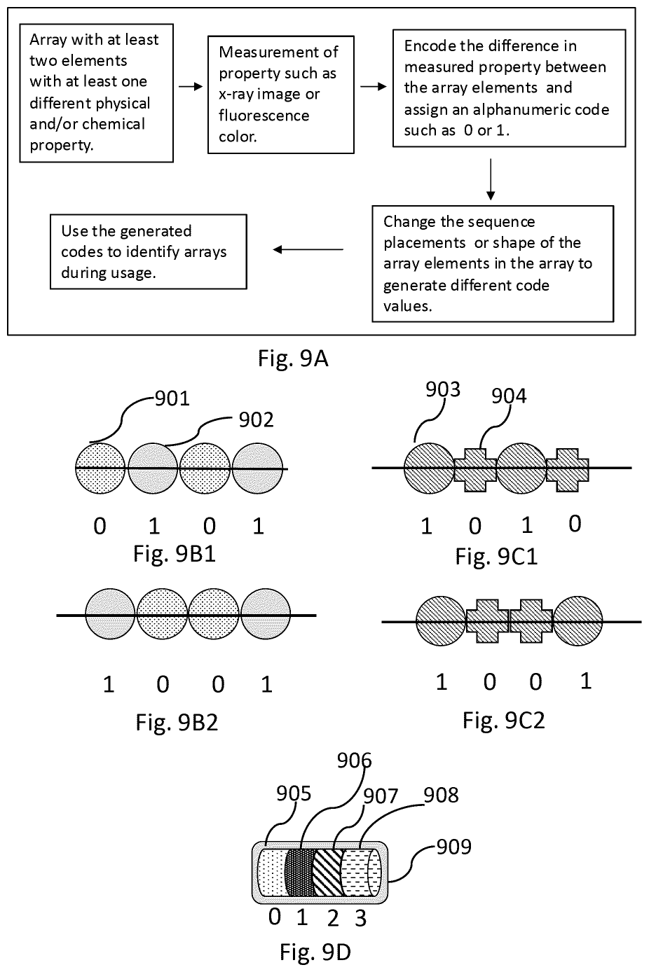

FIGS. 9A to 9D highlight various methods of encoding unique information within arrays by leveraging the sequence of elements and their physical properties to generate numeric or alphanumeric codes for identification, according to one or more embodiments.

FIG. 9A outlines the process where an array with at least two elements differing in properties such as x-ray absorption, fluorescence, or shape and the like is subjected to measurement, assigning numeric values (e.g., 0 or 1) to each element based on their distinct properties.

FIGS. 9B1 and 9B2 demonstrate encoding information in an array based on x-ray absorption properties. This method allows for the internal structure of the array to be visualized and decoded using standard medical imaging techniques.

FIGS. 9C1 and 9C2 illustrate encoding information based on the shape of array elements that is detectable by the unaided human eye or an x-ray image. This approach enables easy identification and verification of the array configuration enabling it to identify after its implantation,

FIG. 9D depicts an interconnected disk array without threads, where elements (905-908) with distinct properties are encased in biocompatible materials (909) like high-density polyethylene or polymethyl methacrylate, approved for long-term implantation. These casings do not interfere with imaging, and properties are encoded as values such as 0, 1, 2, or 3 for unique identification, enhancing the functionality and applicability of the device in clinical settings.

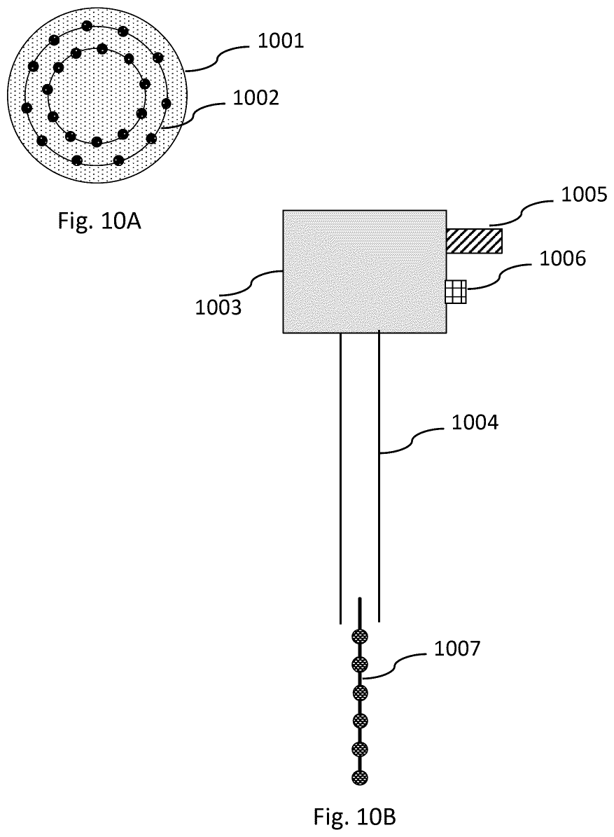

FIGS. 10A and 10B illustrate an ophthalmic device, such as a contact lens embedded with an array device, and a minimally invasive surgical (MIS) device used for deploying and/or retrieving the array device.

FIG. 10A schematically illustrates a contact lens or corneal shield (1001) embedded with an array device (1002) as described in this invention. This design highlights the potential for incorporating drug delivery systems directly into ophthalmic devices, allowing for localized treatment and sustained drug release at the site of application.

FIG. 10B illustrates a minimally invasive surgical (MIS) device equipped with operational components designed to perform various surgical tasks. These components may include instruments for deploying MIS tools, integrated cameras, or cleaning fluid delivery lines. The array device is stored in a spool (1005) and deployed through a trocar or tube (1004) via a pulley mechanism (1006) that enables precise control over the movement of the array. The spool and pulley system facilitates forward and backward motion at the rate of 0.1 mm per minute or more, preferably at 0.1 mm to 500 mm per minute, allowing the array to be extended or retracted as needed during the procedure. The array device is specifically designed to fit within the dimensions of the trocar (1004), ensuring seamless deployment and retraction, highlighting its utility in surgical interventions and patient care.

### DESCRIPTION OF PREFERRED EMBODIMENTS

Interconnected Particle Arrays Pendant or Garland Like Drug Delivery Devices Made from Hydrogels or Organogels.

This invention discloses interconnected particle array devices comprising particles or microparticles connected by a flexible thread, fiber, or suture. Several array compositions, their designs, and methods of preparation and their applications are disclosed.

An overview of various embodiments disclosed in this invention is summarized below. Several illustrative embodiments provide preferred methods for the synthesis of precursors and effective polymerization techniques, including free radical polymerization and condensation polymerization. The preferred methods utilize polyethylene glycol-based macromonomers and precursors to produce biostable and biodegradable crosslinked materials, hydrogels or organogels. Certain embodiments disclose methods for preparing organogel or hydrogel array elements by crosslinking natural or synthetic macromolecules, such as albumin or gelatin, using crosslinkers like glutaraldehyde. Additionally, literature references are provided for alternative precursors and macromonomers applicable to array element preparation. Another embodiment describes preferred methods for fabricating composite microspheres, which can be embedded within array elements for controlled drug delivery applications, where the drug carrier within the composite microspheres may be solid, liquid, or gel form. Further embodiments teach preferred methods for producing beads, with or without holes, suitable for array preparation, and these beads may be coated with drug delivery compositions for enhanced functionality. Several embodiments disclose array preparation techniques involving the insertion of threads from various directions to fabricate array devices of diverse shapes and sizes, including configurations where two threads are incorporated within a single bead or disk to create sheet-like arrays.

Additional embodiments reveal casting methods for precursor solutions into threads, achieving desired sizes, shapes, and sequences to produce arrays with elements that are partially, substantially, or completely embedded within the thread. These casting methods are applicable to both crosslinked and non-crosslinked biostable or biodegradable compositions. Certain preferred embodiments integrate drug carriers within array elements to modulate drug release rates, wherein the drug is encapsulated in the array element material, the drug carrier, or both. In alternative embodiments, drugs are loaded post-formation of array elements through solvent diffusion methods, which can also be employed for the entire array device. Some embodiments focus on drug incorporation exclusively within array elements, while others encompass drug loading in both array elements and threads. Additional embodiments teach the preparation of devices wherein array elements contain drug-loaded microspheres within their matrix, and certain embodiments incorporate live cells or active enzymes encapsulated in array elements.

Various array designs are disclosed, utilizing variables such as the chemical composition of array elements or threads, array element and thread size, type of thread (twisted, braided, or monofilament), array element shape and color, drug concentration in array elements or threads, number and arrangement of array elements, sequence of elements, extra thread length at either end, device compressibility and expandability, porosity of array elements, moisture or solvent content of elements, mobility of array elements within the array, number of knots and thread splicing between elements, drug release rates, and combinations thereof in any proportion to create a wide range of devices for medical or non-medical applications. Preferred array designs include arrays with one or two terminal threads, arrays with cylindrical or spherical elements, arrays where some or all elements are in contact with its neighboring elements, arrays with equal or variable thread lengths per element, arrays featuring both migration-resistant and/or freely movable elements or their combination in any proportion, arrays compressed to reduce overall size, arrays containing one or multiple drugs or visualization agents, and arrays with elements that release drugs at different rates.

The surgical array elements (particles) can be single particle array like shown in FIG. 1F or FIG. 1G. The surgical array disclosed herein can also be single chain like array format wherein multiple particles connected by a flexible thread like shown in FIG. 2A, FIG. 2B, FIG. 2B1 and FIG. 2C. The surgical array can also be configured as a 2-dimensional matrix like shown in FIG. 2F and FIG. 2H wherein particles are arranged in a matrix format and are interconnected by a flexible thread. The surgical array can also be configured as a 3-dimensional matrix like the cylindrical shape shown in FIG. 2I. The surgical array disclosed herein can be used for local or systemic controlled drug delivery. The array disclosed in also can be used in non-medical applications like production of cell or enzyme-based therapeutics or compounds wherein elements of array are used to encapsulate cells or enzymes or as a column material for chromatography. FIG. 1 shows a partial schematic representation of various particles-based arrays that were configured and/or connected with each other to create a surgical array device that can be attached/immobilized at a desired location in the body. FIG. 1A shows a disk shaped cylindrical particle (top view), preferably biodegradable particle capable of releasing a drug in controlled manner over a period or 1 hour to 3 years. FIG. 1A1 is similar to FIG. 1A but is reinforced with biodegradable or biostable mesh or fiber matrix (101) to improve its tear resistance during suturing or threading. FIG. 1B1 is similar to FIG. 1A but contains a hole (102) preferably located at the center of the body. The diameter of the particle (dl) is defined as the diameter of the particle, while the diameter of the hole (d2) is defined as the diameter of the hole.

FIG. 1B2 and FIG. 1B3 are similar to FIG. 1B1 but contain two and five holes, respectively. FIG. 1C presents a side view of FIG. 1B1, illustrating the central hole (102) aligned along the axis of the cylindrical body (103) with a height of h1. The diameter of the hole (d2) is less than the average diameter of the particle (d1). The percentage ratio of hole diameters (R1, d2/d1*100) within the particle can range from 99.99 to 0.1%, with a preferred range of 99 to 1%. The d1-d2 provides wall thickness and it may range from 10 microns to 1 mm depending on the intended use. The diameter of the hole in the array particle (d2) is preferably greater than 10 microns, with a range of 10 microns to 5 mm. The particle size in the array is generally dependent on the desired percentage ratio of hole diameters. FIG. 1D is similar to FIG. 1C except it is partially or completely coated with a composition comprising drug/visualization agent (105) capable of releasing a drug in a controlled manner or assisting in imaging of the product. FIG. 1E is similar to FIG. 1C except it has spherical shape and a central hole (102). FIG. 1F is a drug delivery device made using particles like shown in FIG. 1A to E and a fiber or thread or suture. A suture (107) is threaded through the body of FIG. 1A and/or through 102 hole or center body and has suture thread on either end (107) which is used for attaching or sowing to the tissue or skin. FIG. 1G is similar to FIG. 1F except with knots or migration barriers (108) on the suture that prevent movement/migration of the bead/particle along the suture/thread axis. FIG. 1H is a device made by combining/joining two or more of devices shown in FIG. 1F or G and 109 is the attachment point like a surgical knot or adhesive joint on the suture used for combining the devices. FIG. 2 shows a partial schematic representation of various configurations of an array of drug delivery devices made using biostable or biodegradable suture or thread and drug delivery elements/particle/s are attached to the thread. FIG. 2A shows an array device comprising cubical shaped beads or microparticles threaded/embedded in a suture/thread. 201, 202 and 203 represent three different drug delivery compositions comprising different particle compositions and/or drug/visualization agents. 204 shows an excess of thread on both ends of the device which can be used for attaching/immobilizing the device to the body or skin or tissue for local or systemic sustained drug delivery. FIG. 2B is similar to FIG. 2A wherein two spherical/disk shaped particles instead of cubical particles are used and their sequence is repeated in an alternate fashion three times. One or more particles in array shown FIG. 2B are in close contact with each other wherein the surface of at least one particles touches with the neighboring particle. FIG. 2B1 is similar to FIG. 2B except thread/fiber/tube used (2041) is hollow in nature. FIG. 2C is similar to FIG. 2B except there is a desired amount of space (206) between the particles and the particles are connected via thread (205) through the space 2011. The length of thread between the particles is shown as x1 and the length of thread at the terminal end is shown as x2. FIG. 2D is similar to FIG. 2C wherein the thread is connected through the center of the disk-shaped particle (parallel/aligned to the cylindrical axis) and 205 represents the thread between each particle. FIG. 2E is similar to FIG. 2D except a mechanical barrier such as thread knot (207) is placed at both ends of each particle to prevent migration of particles along the thread. FIG. 2D1 is a folded configuration of FIG. 2D where the space 206 is substantially minimized thus making the device D in a compact shape/form which then can be delivered at a desirable location in the body using a minimally invasive surgical device and expanded (partially or completely) in situ inside the body before implantation. FIG. 2F is a sheet-like structure formed by combining/joining several devices shown in FIG. 2C using 204 terminal ends and an extra thread (208) on both sides. FIG. 2G is a rectangular grid/matrix of suture/thread mesh with length L1 and width W1. The junction where two fibers/threads intersect (209) in the matrix where array elements are attached. X3 and Y3 represent length of suture between two 209 points along length and width of matrix respectively. FIG. 2H shows a device made using fiber mesh shown in FIG. 2G wherein microparticles/disks (201 and 202) are placed at the junction of two fibers intersection point (209). FIG. 2K is a cylindrical version of FIG. 2H wherein particles in the device are arranged in a cylindrical format and z3 shows length of suture between two intersection points (205) along z direction. FIG. 2J shows a schematic of an array containing a disk-like element (210) like biodegradable hydrogel that is separated by solid rigid hollow/solid cylindrical biodegradable plastic tube or bead like PLGA tube or bead (211). 204 shows the thread portion of the device.

The rigid tube 211 facilitates the locking of the position of 210 hydrogel elements within the array, thereby enhancing its migration resistance. Additionally, the 211 tube or particle, that may be devoid of a drug, serves as a spacer, enabling the adjustment of the drug concentration/dosage within a given array device. Utilization of a higher number of 211 elements in a device will result in a reduction in the overall drug concentration within the device.

FIG. 5B and FIG. 5C show illustrative images of implantable array-like devices made using compositions and methods described in this invention. The implantable array-like devices in FIG. 5B and FIG. 5C are made by threading hydrogel disks or casting hydrogels in situ in the thread matrix. The use of hydrogel in these examples is for illustration only and other materials in the array also can be used. Hydrogels disks of desired size and shape were made first by casting a precursor solution in a mold cavity. In one illustrative embodiment, photopolymerizable precursor solutions with different color additives were first made. The solutions were filled in 3 mm diameter and 1 mm depth mold cavity and exposed to light to polymerize and crosslink the precursors. After effective polymerization, crosslinked hydrogel disks were removed from the mold. Several disks or beads with different chemical compositions, sizes and shapes were made.

A 4.0 cat cut suture with the needle was threaded through the disks from the side. The disks were threaded from the side (FIG. 5A1) with minimum or no distance between disks. The sequence of beads was repeated 3 times to make a 12 bead/disk linear array-like device. A suture length of about 1 to 2 cm or more (FIG. 2C, 204, x2) was left on both sides which can be used for attaching the device to the body upon implantation. In another variation of the array device, disks were inserted in the suture and a distance of about 3 to 5 mm was kept between the disks (FIG. 2C, x1=3-5 mm). A suture knot (207) is placed before and after each disk insertion to prevent disk migration in the array. Finally, about 1 to 10 cm long suture thread is left on both sides of the array device for attaching the device. In this configuration, the thread between the disk offers flexibility to the array device so that the device can be fitted around a complex geometry of tissues in the body. In the preferred configuration, array has thread between the beads and at the terminal end/s. Either one both features (terminal thread and between the beads) can be used to securely attach the device at a desired location in the body using standard surgical sutures. The beads are locked in its respective position in the array due to placement of knots or other means.

In the preferred configuration, at least terminal beads are locked or migration resistant, even more preferably almost all beads are migration resistant, even more preferably at least 50 percent or more beads are locked for migration resistance. In another modification, a mesh like device was prepared (FIG. 5C) wherein two sutures are threaded in a single disk/bead to make a mesh like device. In this configuration, the disk is preferably at the intersection of two threads (312). FIG. 5A1 shows a disk shaped cylindrical implant for controlled drug delivery. A suture is threaded through the side wall (via insertion point 501 and exit point 502, parallel to the circular plane and perpendicular to the axis of cylindrical implant) of 5A1 to add multiple implants on the suture thread. FIG. 5A2 is similar to FIG. 5A1 wherein two sutures or threads are inserted from two sides in the side wall (503 and 501) and 503 is preferably at 90 degrees angle relative to 501 on the disk surface. 90 degrees is a preferred angle and other angles between 5 to 89 degrees can be used without any limitation. FIG. 5A3 shows an alternate way to thread the suture wherein the suture is threaded via a central point in the circular plane (504) and exits to the other side of circular plane via central point (505). The terminal length of a thread (FIG. 2, 204) is preferably 1 mm or higher, preferably 0.2 to 60 cm and most preferably 1 cm to 50 cm. In some embodiments a hollow fiber or tube (2041) is used to make an array. The diameter of hollow tube may range from 10 microns to 5 mm, preferably 100 microns to 2 mm. The thread used in making such devices could be made from metal, polymer, ceramic or glass. Polymeric thread, filament or fiber is most preferred. Preferably thread is made from biostable or biodegradable macromolecules or fiber or prosthetic implantable tissue material. Thread used in these devices can be made from single or multiple fibers that can be twisted or braided combination thereof in any proportion can also be used. Threads/fibers can be woven, knitted, twisted or combination thereof to create two- or three-dimensional structures or textiles. A monofilament material can be used in place of thread. Threads made from biodegradable suture materials like polylactide, polyglycolide, polycaprolactone, polydioxanone, polytrimethylene carbonate and their copolymers are preferred. Biodegradable thread can be obtained from animal tissue. Preferred thread is sourced from animal tissue used in gut suture manufacturing which include but not limited to: sheep (ovine) intestines; goat (caprine) intestines; cattle (bovine) intestines; horse (equine) intestines; and pig (porcine) intestines. The submucosal layer of the small intestine is generally preferred because it is rich in natural collagen. When making a thread from animal tissue, animal tissue is procured and cleaned. In some cases, it can be cleaned to remove cellular debris also known as decellularization process. Generally, decellularization involves treating the tissues with detergents or organic solvents. Thread made from decellularized tissue is preferred. Since submucosal layer is a thin tissue, it is generally twisted to make thread of desired size/diameter.

The animal tissue used in the thread is preferred to be non-crosslinked. In making the animal tissue thread, it is preferred that the tissue is never exposed to temperature greater than 60 degree C. which is below its shrink temperature. The exposure of tissue above 60 degree C. will denature collagen (loss of triple helix structure of collagen) and make it unsuitable for implantation use. For additional details about animal tissue based compositions and its processing, please refer to U.S. Pat. No. 7,919,112 and references therein, cited herein for reference only.

Many biodegradable natural and synthetic polymers and biostable natural and synthetic polymers can be used as thread materials. Preferred natural and synthetic biostable polymers for thread include but are not limited to: polyethylene, polypropylene, polyethylene terephthalate, polytetrafluoroethylene, silk, stainless steel and the like.

The preferred diameter of thread is more than one micron and preferably can range from 1 micron to 2000 microns, most preferably 10 microns to 1000 microns. In some embodiments, the preferred diameter can also be expressed as surgical suture in USP sizes from 0 to 4 and from 10-0 to 2-0. The average size/diameter of array elements like bead or disk is preferably more than the average diameter of the thread, preferably 5 percent or more, even more preferably 25 percent or more. The ratio of array element to thread diameter can range from 1.1 to 200, preferably 1.25 to 150.

In places where organic solvents are used in preparation of arrays, gut suture thread or thread made from human/animal implantable tissue is most preferred due to its resistance to dissolution in commonly used organic solvents. The enzymatic degradation pathway of gut suture may be also useful in using hydrated hydrogels (wherein the array element has substantial amount of water relative to its dry weight) such as gelatin or collagen hydrogel as array elements. In the synthetic suture thread, polyglycolate based materials are preferred due to its chemical resistance to many commonly used organic solvents. The combination of suture or thread and solvent for a precursor must be chosen such that physical and chemical properties of thread are not affected or substantially unaffected. Properties such as mechanical strength and its biodegradation profile are substantially unaffected.

In some embodiments, instead of using thread or thread mesh, a film or film strip made from biostable or biodegradable polymer film is used. Prosthetic tissue sheet like material like bovine pericardium or sheep or porcine submucosa tissue and the like also can be used in place of film. The prosthetic tissue material can also be obtained by culturing animal/human cells or tissue engineering methods known in the art. Preferably the film is perforated to create woven fiber like structure like shown in FIG. 2G. The precursor composition is then deposited using methods disclosed in this invention or inkjet printer-like device at a desired place on the perforated film and then crosslinked/cured to make an array-like device. In this device, the thread or thread mesh is replaced with the flexible perforated film for film strip or textile or prosthetic tissue sheet.

Many methods can be used to create a perforated film like mechanical cutting, embossing or stamping, laser cutting, selective etching or dissolution of film and the like. Many holes of various sizes and shapes can be created to create a perforated film. The shape of holes that creates perforation include but are not limited to triangular, rectangular, circular, pentagonal, hexagonal and the like. The film material includes many natural or synthetic polymers as described elsewhere in this specification. Preferred film materials include but not limited to: gelatin or its derivatives, collagen or its derivatives, polyvinyl alcohol or its derivatives, hyaluronic acid or its derivatives, cellulose derivatives like hydroxypropyl cellulose, hydroxymethyl cellulose, polyhydroxy acids, polylactones and their copolymers, polylactide and its copolymers, polycaprolactone and its copolymers, PLGA, sheet materials made from prosthetic human/animal tissue like pericardial tissue or submucosa tissue, human/animal tissue engineered sheet material and the like. The prosthetic tissue used can be biodegradable or biostable. The prosthetic tissue used can be crosslinked or non-crosslinked. Biostable prosthetic tissue is preferably obtained by crosslinking of tissue with glutaraldehyde.

The arrangement of array elements in beads can be in close packing format, with the minimum distance between elements or in close contact with each other (FIG. 2B). In the preferred mode, the thread length (x1) between the elements is the same or substantially the same in the array device. Up to 60-100 percent beads can have same thread length. In some array configurations two or more thread lengths may be used (FIG. 8C, x1 and x1−1 as an example) to construct an array. The arrangements of different lengths in the device may vary depending the array design used. For example, the higher length x1−1 may be used only one time as shown in FIG. 8C or it may be repeated with x1 length in an alternate fashion. The distribution of x1 and higher or lower thread length (x1−1) may vary from 1 percent to 99 percent preferably 50:75, 50:50, 75:50 percent. For illustration, we have used two array lengths, however it is not a limitation. 3, 4, 5 or more types of thread lengths between array elements also can be used. If an array is constructed using two different beads with distinct physical or chemical properties, their sequence can be varied in numerous ways. Physical properties and chemical properties include, but are not limited to, array element shape, size, appearance (such as color, texture, or opacity), porosity, chemical composition, swelling capacity, solvent or water content, crosslinking status, drug type and its concentration, and the like. The sequence can be symmetrical, asymmetrical, random, or a combination thereof in any proportion. Symmetrical sequence with equal particle distances is preferred. The preferred sequence exhibits a symmetrical pattern. Alternate sequences that are repeated one, two, three, or several times are most preferred.

The array particles/elements like disks/beads can be closely packed like shown in FIG. 2B or can be packed with a desirable length of thread between the array elements or disks (FIG. 2C, FIG. 2D and FIG. 5B). In the closely packed arrangement, it is preferred that to beads surface touch with the neighboring beads. The preferred thread length between two array elements (FIG. 2, X1, X3, Y3 and Z3) is 0.5 microns or higher. Preferably the length can vary from 0.5 micron to 30 mm, even more preferably 1 micron to 20 mm. The thread length at the terminal end (FIG. 2, X2) may be 1 mm or longer, preferably may range from 1 mm to 300 mm, preferably 2 mm to 200 mm. The circular shaped disks used in the array are for illustration only. The particles/elements of an array like beads or disks can have many sizes and shapes. The preferred average diameter/size of the bead/disk can range from 0.5 microns or higher. Preferably 1 micron to 15 mm, preferably 1 micron to 10 mm, most preferably 5 microns to 8 mm. The shape of the array element disk or bead can be symmetrical and non-symmetrical or their combinations in any proportion. Symmetrical shape is preferred. The shapes of the array elements, which can be disks or beads, encompass a wide variety of geometric forms. These include, but are not limited to: cylindrical, cubic, cuboid, conical, spherical, elliptical rectangular prismatic, triangular prismatic, hexagonal prismatic, square pyramidal, rectangular pyramidal, triangular pyramidal, hexagonal pyramidal, ring-shaped, toroidal or torus shaped, and similar configurations and their combinations in any proportion. The number of particles/elements in the array can depend on the size of particles and final size of the array. The array device can have 1, 2, 3, 4, 5, 6, 7, 8, 9, 10, 11, 12, 13, 14, 15, 16, 17, 18, 19, 20 or more particles/elements. The number of particles/elements in the array device can vary from one to several thousands, preferably 2 to 2000000 or more. The matrix-like array like shown in FIG. 2F, FIG. 2H, and FIG. 5C can have many sizes and shapes. For sheet like or 3 dimensional array devices, the length (L1), width (W1) and height (H1) can vary from 50 microns or higher, preferably from 100 microns to 500 mm. The shape of the matrix array can be symmetrical or non-symmetrical or their combination in any proportion. Symmetrical shape is preferred. Preferred shapes of matrix include but are not limited to: triangular, rectangular, pentagonal, hexagonal and the like. The array disks can be made from single layer or multilayer unibody materials like described in the related patent application. Preferably the number of layers can vary from 1 to 5. The preparation of two or more layered particles or disks have been disclosed earlier in the related patent application. The array elements such as beads or disks can be made from polymeric, ceramic, glass or metal-based materials. Polymeric materials are preferred. Polymeric materials used could be biostable or biodegradable. Biostable hydrogels could be more applicable to cell/enzyme encapsulation applications which are used to manufacture high value chemicals or pharmaceuticals. Monomers utilized in the synthesis of bistable hydrogels encompass a diverse range of monomers/compounds, including but not limited to: acrylamide, methacrylamide, acrylic acid, methacrylic acid, 2-hydroxyethyl methacrylate, 2-hydroxy propyl methacrylate, n-vinyl pyrrolidinone, and macromonomers derived from polydimethyl siloxane, polyethylene oxide derivatives, and polyethylene glycol derivatives. These monomers can be combined in various proportions with other monomers known in the art to form homopolymers or copolymers. In controlled drug delivery applications, biodegradable substances are the preferred choice.

Hydrogels and organogels are particularly favored materials. The most desirable arrays are constructed from hydrogels or organogels based on synthetic polymers, natural macromolecules, or those that break down through enzymatic processes in the body. It is possible to utilize combinations of these hydrogels in conjunction with threads composed of synthetic or natural polymers, or prosthetic tissue. Following combinations of thread and hydrogels/organogels is preferred and include but not limited to: synthetic polymer thread and synthetic polymer hydrogel/organogel, synthetic polymer thread and natural polymer hydrogel/organogel, synthetic polymer thread and enzymatically degradable hydrogel/organogel, natural polymer thread and synthetic polymer hydrogel/organogel, natural polymer thread and natural polymer hydrogel/organogel, natural polymer thread and enzymatically degradable hydrogel/organogel, prosthetic tissue and synthetic polymer hydrogel/organogel, prosthetic tissue and natural polymer hydrogel/organogel and the like. The thread used in combinations as discussed above may be either loaded/coated with drug or may not contain any drug. The preferred thread is used in above combinations is colored. Organogels whose preparation generally involve use of organic solvent are preferably used in combination with natural threads like gut suture. Biodegradable hydrogels or organogels are most preferred for implantable devices or controlled drug delivery applications. In one embodiment an illustrative biodegradable plastic polylactide was used to make the bead or disk. Since such beads require high temperature processing like injection molding or extrusion and the like, it is preferred that such disks are coated with organogel or hydrogel or biodegradable polymer composition without dissolution of the bead. Other biodegradable polymer plastic beads can also be used in place of plastic polylactide. The preferred biodegradable polymers are listed in the defined biodegradable polymer part of this application. The plastic beads preferably have holes like shown in FIG. 1B1, FIG. 1B2 and FIG. 1B3. Beads and array elements can also be made from biodegradable metals or glass known in the art. The biodegradable metal used could be any metal that degrades safely in the body in a period from few days to few years, preferably in 7 days to 270 days. Many alloys of calcium, magnesium, aluminum, zinc, manganese, iron and other metals are known to degrade safely upon implantation. Preferred polymers or metals that do not create extreme basic or acidic local environments upon implantation. Preferred metals or glasses that are safely removed by the body via biodegradation process like hydrolysis, slow dissolution or other corrosion type mechanisms.

Metal alloys based on magnesium are preferred due to its long history of use. When metal, plastic, ceramic or glass based beads are used, it is preferred that such beads are coated or infused with biodegradable controlled drug delivery compositions such as shown in FIG. 1D.

In one embodiment, biodegradable polymer such as PLGA are procured as solid plastic beads and those beads are further processed to make array devices. In one illustrative embodiment, biodegradable beads or disks are prepared from commercially available biodegradable polymers or plastics such as poly(lactic-co-glycolic acid) (PLGA), polylactide (PLA), PEG-polylactone copolymers and the like. The beads can be manufactured through various methods, including extrusion, injection molding, and 3D printing and the like. For example, 2 mm diameter rods of PLGA (50:50) are extruded and cut into disks of 2 mm in height. Alternatively, low-melting biodegradable polymers like PEG-polylactone copolymers can be melted in molds or sintered from powders to produce beads of desired shapes and sizes, including configurations with central holes for threading. The beads can be further modified by incorporating holes using micro-drilling or laser drilling techniques, facilitating ease of threading onto sutures. The surfaces of these beads can be coated with drugs or visualization agents using crosslinked or non-crosslinked polymer coatings. Crosslinked polymer coatings can be achieved through free radical polymerization using PEG-based macromonomers or via condensation polymerization methods involving PEG-tetramine and NHS-ester terminated PEG derivatives. Crosslinked coatings may carry a drug carrier such as PLGA biodegradable polymer to assist in controlled release of drugs.

For non-crosslinked polymer coatings, synthetic biodegradable polymeric carriers such as PEG-polylactone copolymers are utilized, wherein the beads are incubated in drug-loaded organic solvent solutions, followed by drying to achieve a uniform coating. Natural non-crosslinked carriers, like gelatin, are employed by dissolving gelatin in hot water, incorporating therapeutic agents such as chlorhexidine, and subsequently incubating the beads in the gelatin solution with continuous stirring. After coating, the beads are dried through air drying or lyophilization. In another embodiment, a coating material that uses a synthetic biodegradable polymer is used.

Such coatings degrade by hydrolysis of ester bonds. In another embodiment, a natural polymer coating that degrades by enzymatic pathway upon implantation (gelatin) is used as a coating material. The preferred method of coating are spray coating or dip coating, but bother coating methods known in the art that provide uniform coatings also can be used without limitation. The coating is done on the array elements or beads before array device is made or after array device has formed. The preferred coating thickness is preferably greater than 5 microns. The coating thickness may range from 5 microns to 2 mm, preferably between 10 microns and 1 mm. Drug concentration on the coating may range from 0.1 percent to 40 percent relative to dry coating weight, preferably 0.5 percent to 30 percent. Biodegradable polymers that form uniform film via solvent casting are preferred. In some embodiments, crosslinked or non-crosslinked polymer beads are exposed to a mixture of solvent and non-solvent so that hard plastic bead can be softened via solvent exposure without dissolution. A list of solvent and non-solvent can be found in polymer handbook. Experimental conditions such as type of solvent and non-solvent used, their composition in a mixture, temperature, time of exposure and pressure can be altered to achieve a desired softness that is suitable for threading. In one embodiment, biodegradable polymer such as PLGA are procured as solid plastic beads and those beads are further processed to make array devices. In one illustrative embodiment, biodegradable beads or disks are prepared from commercially available biodegradable polymers or plastics such as poly(lactic-co-glycolic acid) (PLGA), polylactide (PLA), PEG-polylactone copolymers and the like. The beads can be manufactured through various methods, including extrusion, injection molding, and 3D printing and the like. For example, 2 mm diameter rods of PLGA (50:50) are extruded and cut into disks of 2 mm in height. Alternatively, low-melting biodegradable polymers like PEG-polylactone copolymers can be melted in molds or sintered from powders to produce beads of desired shapes and sizes, including configurations with central holes for threading. The beads can be further modified by incorporating holes using micro-drilling or laser drilling techniques, facilitating ease of threading onto sutures. The surfaces of these beads can be coated with drugs or visualization agents using crosslinked or non-crosslinked polymer coatings. Crosslinked polymer coatings can be achieved through free radical polymerization using PEG-based macromonomers or via condensation polymerization methods involving PEG-tetramine and NHS-ester terminated PEG derivatives. Crosslinked coatings may carry a drug carrier such as PLGA biodegradable polymer to assist in controlled release of drugs. For non-crosslinked polymer coatings, synthetic biodegradable polymeric carriers such as PEG-polylactone copolymers are utilized, wherein the beads are incubated in drug-loaded organic solvent solutions, followed by drying to achieve a uniform coating. Natural non-crosslinked carriers, like gelatin, are employed by dissolving gelatin in hot water, incorporating therapeutic agents such as chlorhexidine, and subsequently incubating the beads in the gelatin solution with continuous stirring. After coating, the beads are dried through air drying or lyophilization. In another embodiment, a coating material that uses a synthetic biodegradable polymer is used. Such coatings degrade by hydrolysis of ester bonds. In another embodiment, a natural polymer coating that degrades by enzymatic pathway upon implantation (gelatin) is used as a coating material. The preferred method of coating are spray coating or dip coating, but other coating methods known in the art that provide uniform coatings also can be used without limitation. The coating is done on the array elements or beads before array device is made or after array device has formed. Biodegradable polymers that form uniform film via solvent casting are preferred. In some embodiment, polymer beads are exposed to a mixture of solvent and non-solvent so that hard plastic bead can be softened via solvent exposure without dissolution. A list of solvent and non-solvent can be found in polymer handbook. Experimental conditions such as type of solvent and non-solvent used, their composition in a mixture, temperature, time of exposure and pressure can be altered to achieve a desired softness that is suitable for threading.

Hydrogels or organogels are most preferred array elements due to their soft nature and ease of suturing and high biocompatibility. Bead or disk preparation methods that use mild processing conditions like ambient temperature (15 to 40 degree C.) or pressures (atmospheric pressure) are most preferred. Such mild conditions (temperature, pressure and use of standard organic or aqueous solutions) are especially useful for encapsulation of live cells, drugs/bioactive compounds in the array particles/elements. Fast polymerization and crosslinking (crosslinking within 60 minutes, preferably within 5 minutes) of precursors is highly desirable and many embodiments disclosed in this invention crosslink under 2-10 minutes under ambient conditions.

One of the main advantages of organogels and hydrogels used in this invention is that the formation and drug incorporation is done under mild conditions like between 0-45 degree C., preferably around 10-40 degree C. and in a short amount of time. Many drugs can only tolerate mild processing conditions. If melt processing conditions like injection molding or extrusion are used, then it is preferred that polymer melting point is below 100 degree C. preferably below 60 degree C. In some illustrative embodiments drug compositions can be coated on the elements of the array. The hydrogels or organogels used in array preparation can be crosslinked or non-crosslinked. Hydrogels and organogels comprising polyethylene glycol or its derivatives like PEG-polylactone copolymers, collagen or its derivatives, cellulose or its derivatives, polyvinyl alcohol or its derivatives, hyaluronic acid or its derivatives, chitosan or its derivatives, and albumin and their combination in any proportion are preferred. The hydrogels or organogels can be biostable or biodegradable. The hydrogels or organogels used in the array can be hydrated or solvated or can be partially or completely dry. During array making processes like threading, solvated/hydrated materials are preferred due to their softness and ease of threading. Due to weak mechanical properties of many hydrogels or organogels are susceptible to breakage or tearing during threading or during the use of the device. The tear resistance is improved by reinforcing the hydrogel or organogel beads/elements using fibers or filaments or fiber mesh or woven/knitted fiber structures (FIG. 1A1). Biodegradable materials for hydrogel reinforcement are most preferred. Hydrophilic fibers like collagen fibers, cellulose fibers, protein based synthetic or natural fibers, prosthetic tissue or gut suture materials are preferred materials for hydrogel reinforcement. Hydrophobic biodegradable polymeric materials like materials used in synthetic sutures can also be used for reinforcement. The amount of water or solvent in the disk or bead at the time of threading or embedding can be 5 percent relative to total weight of disk or higher. The preferred amount of water or organic solvent in the disk or bead can range from 1 to 99 percent, preferably 5 to 95 percent. The preferred solvent in the organogel is biocompatible and water soluble. The preferred organic solvents that can be used but are not limited to: ethanol, isopropanol, glycerol, polyethylene glycol or its derivatives preferably with molecular weight less than 1000 g/mole, dimethyl sulfoxide, n-vinylpyrrolidone and the like. Some implantable array devices can have organogel biodegradable array elements that are partially or completely swollen in biocompatible water soluble organic solvent or their mixtures with desired amount of drug. Preferably such organogels are crosslinked materials. Upon implantation, the organic solvent is dispersed in the tissue leaving a biodegradable array element and drug which is then released in a controlled manner. The array preparation or threading can be done in the manufacturing facility, or it can also be done just prior to implantation in the surgical suit environment. This way the array size, sequence, number of drugs incorporated and the like is controlled by the user of the device. In some preferred embodiments, in situ casting of hydrogels or organogel beads/disks in the thread or the thread mesh has been disclosed. In the insitu casting method, the organic or aqueous precursor solution droplets are added on top of thread or mesh (with or without a mold). After partial or complete immersion of thread in the solution droplets, the crosslinking is initiated. After crosslinking, the crosslinked material is partially, substantially or completely embedded in the thread. Extremely small precursor droplet size/volume is possible due to various methods of droplet generation known in the art and such methods can be used without limitation. In situations where threading of array elements is difficult due to small size or physical properties of the materials, in situ casting of precursor solution on the thread is a preferred method to prepare array devices as described in this invention. In one illustrative embodiment, a microfluidic device based droplet generator was used. Other droplet dispensing devices known in the art also can be used without limitation. The surface coverage of precursor solution with the thread will depend on surface properties of the thread, the surface on which thread is kept for support and solvents/chemical composition used in droplet solution. Since many organic solvents or aqueous solution can be used for droplet generation, a desired amount of surface wetting of thread surface and support material surface can be achieved by the precursor solution. Alternatively, thread or support surface can be modified with surface treatments such a plasma modification or coatings known in the art to achieve a desired wetting of thread surface. The thread diameter and droplet size are controlled to achieve partial, substantial or complete immersion of thread in the droplet solution and then crosslinking is initiated. In some embodiments, a mold cavity is used to achieve a desired shape like disk shape.

Mold cavities of various sizes and shapes can be used to prepare array elements of desired size and shape. In another illustrative embodiment, the solution droplets are frozen first using many methods known in the art. In one illustrative method, solution droplets are first added in cryogenic baths like liquid nitrogen or helium and frozen. The nitrogen gas is evaporated and the collected frozen droplets are added on top of the thread of desirable size in a desired sequence at a desired location in the frozen state. The frozen solution droplets are melted by warming to ambient temperature or to warm temperature (below 60 degree C., preferably below 40 degree C.) and then exposed to crosslinker or exposed to light to crosslink the precursor solution. If gelatin or collagen or albumin solution is used, exposure to glutaraldehyde crosslinker solution (0.2 percent in PBS or water) can crosslink the protein solution. Alternatively, macromonomer solution with photoinitiator is exposed to UV or visible light, gamma radiation or electron beam to initiate crosslinking in the solution and to form an embedded array.

FIG. 6A and FIG. 6C show two additional preferred methods in making arrays from a precursor solution. FIG. 6A shows a method for making an array device using a photosensitive precursor.

FIG. 6A outlines a method for creating arrays from a precursor solution using a photolithography-like method involving a photosensitive precursor. A photopolymerizable precursor such as a gelatin methacrylate solution with a photoinitiator that polymerizes when exposed to 360 nm light (601) is applied over a thread (602) along with a photomask (603) that has a desired pattern of transparent and opaque areas. The precursor solution (601) is applied as a continuous layer along the thread, with the terminal portion of the thread remaining uncovered. Upon exposure to 360 nm light through the transparent portions of the mask (603), the precursor solution polymerizes and crosslinks only in the areas exposed to light, while the unpolymerized portion in the unexposed areas is removed by solvent washing or other methods, leaving behind an array with crosslinked precursor material (604) as shown in panel FIG. 6A2. This process can be repeated to add array elements using the same or different precursor compositions along the remaining portions of the thread. FIG. 6B presents an array where the thread is replaced by a thin strip of array element material, produced using a modified method described in FIG. 6A. This method uses a different photomask wherein white portions (606) between the crosslinked material (605) are shielded from light exposure during the polymerization process. The unpolymerized portion is subsequently washed away to produce a matrix-like array structure without thread but with thin strip of element material as shown in FIG. 6B.

FIG. 6C demonstrates a method for creating an array device where specific portions of the crosslinked materials are selectively removed to form an array structure. A photopolymerizable precursor (601) and thread (602) are provided, with a thin film of precursor solution (601) applied on the thread (602) except at the terminal ends. The precursor is crosslinked (607) embedding the thread except at the terminal ends (Panel 6C.1). Parts of the crosslinked material (607) are then selectively removed using techniques such as laser ablation, mechanical cutting, or selective dissolution/degradation, without affecting the thread, thus creating an array-like structure with thread positioned between two array elements (608, shown in Panel 6C.2). The device such as shown in panel 6C1 itself can be used for implantation. The fiber/thread and materials used in FIG. 6C1 enables to make variety of devices with different controlled drug delivery profiles.

Adding of precursor solution droplets on thread surface in a desired sequence and subsequently crosslinking them is the most preferred way to prepare array devices. Alternatively, commercially available positive photoresists (AZ 5214E, AZ 10XT, Polymethyl methacrylate and the like) and negative photoresists (AZ 125nXT, SU-8, AZ nLOF 2000 and the like) can be used along with appropriate masks to create desired patterns using photolithography methods known in the art. Devices created using such methods are considered part of this invention.

Generally, polymeric carrier and drug are coated on a flat surface using spin coating method. Photoresist is applied on the coated solution and the photoresist is exposed via mask to selectively remove photomask material. Pattern created is used to selectively remove polymeric carrier material to produce a pattern of polymeric carrier. In the preferred mode, photoresist and polymer carrier are replaced by biodegradable photopolymerizable compositions as described in this invention.

In one embodiment, an inkjet printer-like device with ink from the cartridge substituted with precursor solution. FIG. 3D shows a flow diagram for creating an array device using injectable or sprayable liquid precursor compositions. Panel 3D.1 displays a thread, and Panel 3D.2 shows a thread mesh with 25 thread junction points (312). Apparatuses in Panel 3D.3 possess the ability to form fine droplets or spray the desired amount of precursor solution. Panels 3D.31 and 3D.32 illustrate a print head or a syringe with needle (313 and 316), filled with precursor solutions (314 and 317) that can be injected or sprayed via nozzles (315 and 318). The solutions are deposited/sprayed along the thread in a desired sequence (Panel 3D.4) or at the junction point 312 on Panel 3D.2 mesh (Panel 3D.5). The deposited solution is then crosslinked to produce either a single thread array device (Panel 3D.6) or a matrix-type device (Panel 3D.7), wherein the crosslinked compositions (318 and 319) are attached/embedded in the thread or at the intersection of two threads. In one embodiment, a polymer precursor solution (1 microliter to 50 microliter) is manually added on a suture thread using a standard calibrated micropipette device.

A distance of 5 mm is left between each droplet on the suture. The droplets are then exposed to light to produce an array with embedded crosslinked material. Alternatively, droplets from the micropipette device can be added on a fiber matrix (Panel 3D.2) at the junction of intersecting fibers (312). The droplets are then crosslinked to make a matrix like embedded array. Inkjet printer cartridge wherein the ink is substituted with precursor solution 314 or 317. The inkjet printer along with its printing software is used to deposit/spray the precursor solution at a desired location in the thread or thread mesh and then crosslinked. In some embodiments, two precursor solutions (precursors that polymerize by condensation polymerization) are first mixed in situ and then deposited using the inkjet cartridge or micropipette device. Alternatively, cartridges can be substituted with a droplet generator or syringe with needle and one or more generated droplets are added at the desired location and then crosslinked to make an array. The average diameter of droplet size used in depositing the precursor solution may range 0.05 microns to 1 mm, preferably 0.1 micron to 500 microns. The total volume of injectable composition deposited on the thread is preferred be about 0.5 femtoliters or higher, preferably about 10 picolitres or higher and most preferably 1 nanoliter or higher. The total deposited volume per array element may vary from 0.5 femtoliter to 0.5 ml, preferably from 10 picolitres to 0.3 ml and even more preferably from 1 nanoliter to 0.1 ml. The deposited solution is preferred to be in an array format as shown in FIGS. 2 to 3.

In one embodiment, the present invention provides a method for producing an array wherein liquid precursor droplets are initially frozen using a cryogenic bath such as liquid nitrogen or helium. Other cryogenic methods known in the art, including those based on solid carbon dioxide or aqueous solutions with salts, may also be utilized, with liquid gases such as nitrogen gas being preferred. The liquid precursor compositions are introduced into the cryogenic bath, during which the liquid nitrogen evaporates, resulting in the rapid freezing of the droplets. The frozen compositions are then arranged on a thread in a desired sequence and spacing, with or without the assistance of a mold, depending on the intended configuration. The frozen compositions may subsequently be allowed to warm to room temperature, where they return to their liquid state and partially or substantially embed into the thread. Once properly positioned, the liquid compositions are crosslinked and/or lyophilized to lock them in place, forming a stable and functional array. This method ensures precise arrangement and secure attachment of the array elements to the thread, enabling the creation of highly customizable and versatile arrays for various applications.

In one embodiment, the present invention provides a method for producing an array using melted polymer. Polymer with a melting point below 60° C. is preferred. It is preferred that polymer melts below the shrink temperature of gut suture material. A melt-dispensing device is employed to form droplets of the polymer in a suitable size, which are then added onto a thread, either directly or within a mold cavity, depending on the desired configuration. The size of the droplets, their arrangement, and spacing on the thread are selected based on the intended array design. Once positioned, the melted polymer is allowed to partially, substantially, or completely embed into the thread. The polymer is then cooled to solidify and secure its attachment to the thread.

After cooling, the formed array is removed, resulting in a stable and customizable structure suitable for various applications. In one embodiment, commercially available 3D printers that utilize melted polymer to create three-dimensional structures can be adapted for use in the melt method described herein. These printers, which are widely used for precision fabrication, employ controlled heating and dispensing mechanisms to melt polymer filaments or resins. The melted polymer is then extruded through a nozzle to form droplets or layers that solidify upon cooling.

This technology can be leveraged to precisely control the size, spacing, and arrangement of polymer droplets/melts on a thread to produce arrays with high accuracy and repeatability. By programming the 3D printer to dispense the polymer onto a thread, either directly or within a mold cavity, the method ensures consistent placement and embedding of the polymer droplets.

The printer's capability to regulate temperature, deposition speed, and spatial resolution enables precise customization of the array configuration. The melted polymer is allowed to partially, substantially, or completely embed into the thread before cooling and solidifying to form a stable array. This approach combines the precision of 3D printing with the versatility of the melt method, making it suitable for producing complex and tailored arrays for a wide range of applications.

Threading of premade disks or array elements and in situ casting of precursor solution or melts are preferred methods of array preparation. One advantage of in situ precursor casting method is that the embedding of thread locks the position of the array element (disk) in the array and therefore the array device does not need holes or knots, and threading operations to prepare the array device. Threading of premade array elements or disks is generally preferred for the macro size beads/disks with sizes from 0.5 millimeters or more, preferably 0.8 to 1 mm or more. For, micron size array elements like beads/disks, preferably sizes 1 mm or less, in situ casting using precursor or polymer solution is preferably used. Other methods of bead incorporation in the array device known in the art or yet to be discovered can also be used without any limitation. Devices with micro size beads based are generally preferred, but not limited for human therapeutic/surgical applications. Devices with macro size beads are preferred, but not limited for veterinary therapeutic/surgical applications.

In certain embodiments, the array device is designed with a configuration wherein every alternate array element is fixed or immobilized, leaving one or more free, movable elements positioned between the immobilized elements. This arrangement facilitates the easy removal of the free elements from the thread, enabling the user to adjust the drug dosage based on the number of elements retained in the array. The presence of free, removable elements enhances user flexibility and facilitates customization of the therapeutic regimen according to specific clinical needs. Removal of an element creates a segment of empty thread, which can be utilized for attachment to the surgical site or other therapeutic purposes, ensuring the structural integrity of the device is maintained. The alternate placement of immobilized and free elements is provided as an illustrative example, and the configuration may include varying numbers of movable elements—ranging from one to ten or more for larger devices such as devices for human or veterinary use in larger animal. For smaller elements, less than one mm in average diameter, this could 1 to several thousands, preferably 1 to 10000 between immobilized elements. This flexibility accommodates diverse dosing and anatomical requirements. In another embodiment, only the terminal elements are immobilized, with all intermediate elements designed to be free or movable. This configuration ensures ease of removal and reconfiguration while maintaining the stability of the device at its terminal ends. Such adaptable designs provide enhanced utility and versatility for the array device in surgical and therapeutic applications. The disclosed configurations are not limited to the embodiments described herein but encompass all variations that achieve the intended functionality of adjustable dosage and surgical attachment.

Surgical array devices comprising live therapeutic cells are also disclosed in this invention. Such devices are preferred to be made just before implantation. Array elements or disks containing live therapeutics cells can also be prepared first with cells (viability greater than 70 percent) and then used in array-like devices. Array-like devices with live cells can be stored at cryogenic temperatures (liquid nitrogen temperature, −192 degree C.) using cell storage methods known in the art. Typically such methods use a cell preserving agent like dimethyl sulfoxide to maintain cell viability and controlled rate of cooling to achieve cryogenic storage temperatures (liquid nitrogen temperature). In one illustrative embodiment, live cells are first encapsulated in the disks or beads and then used in making the array or necklace-like device. The viability of cells in the disk can range from 30 to 100 percent, most preferably 50 to 99.9 percent. The array elements/disks can have 1 live therapeutic cell to several million cells per array element or disk.

Number of live cells per disk can range from 1 cell to 100 million cells, preferably 2 cells to 10 million cells. Disks or beads with different types of cells in a single device can also be used. In a necklace-like array device, 1, 2, 3, 4, 5 or more types of therapeutic cells can be incorporated. Each disk or bead in the necklace type device can have the same or different type of therapeutic cells depending on the intended use.

In some embodiments, array elements like disks or beads with 1, 2, 3, 4 and 5 or more holes can be used. Disks with 1 to 5 holes are preferred. The holes are preferably used for threading while making the array/necklace like device. The holes in the disks are made while casting the precursor solution or can be made using mechanical or laser drilling or other operations after the bead or disk has been made. The different colors in necklace-like devices represent a different chemical or physical composition and associated physical and chemical properties like drug/visualization agent, crosslinking density, swelling capacity, biodegradation mechanism drug release rate or profile and the like. Color can also represent a drug or bioactive compound, visualization agent, sensor or tag like radio-frequency identification tag (RFID). The array-like devices described in this invention have the ability to release a drug in a controlled manner over a period of 1 hour to 3 years, preferably 3 hours to 2 years and most preferably 6 hours to 1.5 years.

The surgical array-like device disclosed in this invention can exist in at least two forms or configurations. One of the configurations is unmodified or compressed form and the other is expanded form. There can be other configurations between these two configurations. The conversion of the device from unmodified/expanded form to compressed form and reversing back again to an unmodified/expanded form is an important property of this device. The flexibility of the thread within the interconnected array elements enables this flexibility to the device. FIG. 5D shows a schematic of a linear array device wherein several array elements are interconnected by a thread. The device (FIG. 5D) is folded along the length at five folding points (506) to produce a compact, sine wave, or spiral-like shape array (FIG. 5D1). The device (Panel FIG. 5D1) is further compressed along the length to reduce its overall length and to produce a more compact form (FIG. 5D2). The pitch (507) of the device in FIG. 5D2 is less than the pitch of the device shown in FIG. 5D1. The number of folds and pitch length are varied to produce various compact and expanded forms of the array device (FIG. 5D). The preferred array device has ability to fold one or more times. The preferred device can have one or more folding point/s (506). Number of fold or folding points in the device can range from 1 to 100, preferably 1 to 50.

The pitch (507) in a folded device can range from 1 micron to 40 mm, preferably 5 microns to 20 mm. FIG. 2D shows an exemplary array wherein the array has six particles or disks separated by flexible thread (205) and terminal thread 204 for attachment. When this device is compressed along the axis of the thread, the thread between the elements (205) folds (FIG. 2D1, thread is folded) bringing the particles/disks in the array closer to each other forming a compressed form (FIG. 2D1). The compressed form (FIG. 2D1) has significantly less length/volume compared to uncompressed/expanded form (FIG. 2D). If desired, the compressed form can be re-expanded and used. This ability to form compressed form is especially useful in implanting these devices using minimally invasive surgical techniques like laparoscopy, thoracoscopy, arthroscopy, intraluminal endoscopy, endovascular techniques, catheter-based cardiac techniques (like balloon angioplasty), and interventional radiology and the like. In one illustrative example, six 3 mm diameter and one mm height gelatin methacrylate disks were cast and crosslinked into a mold. A surgical suture is threaded through the disk while maintaining 10 mm distance between the disks.

A 10 mm thread on both terminal ends (similar to shown in FIG. 2D) was also added. The device had a total thread length about 76 mm including height/thickness of the disks. This array is compressed along the length of the thread including terminal ends that were also folded. Upon folding, the length of the array device is reduced to about 6-8 mm. Thus, upon compression, the length of the device has reduced from about 76 mm to about 8 mm, about a 9 times reduction in length of the device. The above example is for illustration only. The surgical array devices with expanded form can be compressed 5 percent or more, preferably 20 percent or more, even more preferably 50 percent or more to make a compressed form. The preferred compression ratio (compressed form length/expanded form length*100) is 5 percent to 95 percent, most preferably 10 percent to 90 percent.

FIGS. 7A to 7H display a variety of array configurations with diverse shapes and sizes, each designed to cater to specific functional and therapeutic needs in accordance with one or more embodiments. FIG. 7A shows a linear array with beads, which can be used in various therapeutic applications due to its simple yet versatile design. FIG. 7B depicts a rectangular hollow frame (701), which serves as the structural backbone for more complex array configurations. FIG. 7C and FIG. 7C1 illustrate several linear arrays with hollow tubing or thread (shown as in FIG. 2B1) that are attached to the frame 701. The connection point 702 provides an open fluidic connection between the hollow frame and hollow thread/tube (2041), facilitating the transfer of gases, nutrients, or waste products if hydrogel beads (used in FIGS. 201 and 202) encapsulate live cells or active enzymes. FIG. 7D highlights linear array elements looped around several times within a solid frame (703), with attachment points (704) used to increase the density of beads/elements per frame, optimizing the functional capacity of the array. FIG. 7E shows a single point spiral shaped array in a circular frame (705) with 706 as thread attachment point to the frame. Some of the arrays disclosed in this invention can have a branched structure, as depicted in FIGS. 7F and 1L. The array shown in FIG. 7F comprises a main or central branch (709) and five branches (710) on either side of the main branch. One of the branches contains three array elements (202), while the other branch contains a single terminal array element. The branches can be severed along the cutting plane 711 without affecting other branches. This feature may be advantageous in adjusting a drug dosage, as desired. The branched array depicted in FIG. 7F serves solely as an illustration. Variables such as the number of branches, including the central branch; the number of array elements in the main branch and side branch; the sequence and arrangement of array elements; and similar parameters can be adjusted to suit the intended application. Artisans should recognize that numerous variations of branched array structures are feasible, and these variations are considered integral to this invention. The number of branches in the array can exceed two and range from two to 1000, preferably from three to 500.

FIG. 7G shows a linear array that is spirally placed/wound around tubular organ such as artery (708) or device such as vascular stent or graft. FIG. 7H shows a linear array device in a spiral configuration wherein all array elements are closely packed with each other. This patent is not limited to shapes shown in FIG. 7, 8 or 2. Many 2 or 3 dimensional shapes can be used without limitation and such shapes are parts of this invention. These shapes can be symmetrical or non-symmetrical or any combination thereof in any proportion. The shapes include but not limited to are: cylindrical, cubic, cuboid, conical, spherical, elliptical rectangular prismatic, triangular prismatic, hexagonal prismatic, square pyramidal, rectangular pyramidal, triangular pyramidal, hexagonal pyramidal, ring-shaped, toroidal, and the like.

FIG. 8 schematically represents various array configurations designed to address diverse biomedical applications. These arrays incorporate spherical elements, threads, and functional modifications such as drug delivery and mechanical adaptability. The number of elements in the array, device length and array shape shown is for illustration only and does not limit the invention in any way. Each design emphasizes specific attributes like spacing uniformity, modularity, or controlled material properties. While only a limited number of elements are illustrated for clarity, this is not a limitation, as practical arrays can include more or fewer elements tailored to the intended use. FIG. 8A introduces an array with spherical elements interconnected by a single thread, ensuring uniform spacing (x1) between the elements. The uniformity simplifies device characterization, ensuring predictable spacing and loading during implantation. The inclusion of a terminal thread (204) at one end facilitates tissue anchoring, while the absence of a terminal thread at the opposite end allows flexibility in attaching additional modules or extending the array. This straightforward design is ideal for applications requiring consistent spacing and uncomplicated handling. FIG. 8B extends the concept of FIG. 8A by incorporating terminal threads at both ends, providing enhanced fixation and support in diverse surgical scenarios. This design allows the array to be securely attached to two distinct anchor points, ensuring stability FIG. 8B1 evolves further by connecting both terminal threads at a central point (801) to form a loop, creating a circular or elliptical configurations or their combinations. Such designs are particularly advantageous in applications like vascular grafts, vascular stent or stent graft, where tensioned arrays are essential for structural integrity and function. Another design feature of the array is the incorporation of variable spacing between beads at specific locations (FIG. 8C). In these regions, the distance between two beads is reduced/increased by 10% to 2000% compared to the standard spacing. This longer thread length in designated sections facilitates cutting the array into smaller segments while leaving sufficient thread on each part for secure attachment. This feature enhances the modularity and adaptability of the array for different surgical applications and dosing requirements. This design incorporates at least two distinct thread lengths (x1 and x1−1) between array elements, with x1−1 being approximately twice the length of x1. The variation in spacing allows the array to adapt to complex geometries or functional requirements. For instance, areas requiring greater flexibility or reduced density can utilize longer thread sections. If desired the array can have 2, 3, 4, 5 or more different types of spacing between elements. The ability to cut the segment the array along plane 802 into separate arrays (C1 and C2) demonstrates modularity, enabling device customization for varied medical applications, such as localized drug delivery or tailored tissue integration. It is preferred that array elements are migration resistant and cutting does not unravel some or all the beads in the device. The cutting of array provides the user precisely control the drug dose if incorporated in the array. After cutting, the device still has one or more anchoring threads for tissue attachment. FIG. 8D introduces the concept of arrays loaded with embedded drugs and/or visualization agents. The design leverages color gradients to represent drug concentration variations, with darker shades indicating higher concentrations or vice versa. This controlled loading allows for precise therapeutic or diagnostic functions, enabling gradual drug release or improved imaging contrast. Such arrays could find applications in targeted drug delivery systems or intraoperative visualization aids or loading an analytical agent with known concentrations for calibration and other analytical parameters. FIG. 8E highlights an array with one or more elements (803) larger than the others. The variation in size allows for differentiated functions, such as increased mechanical support or localized drug reservoirs. This design ensures that the array can address specific clinical challenges, like reinforcing weaker tissue regions while maintaining a compact overall structure. It can also indicate certain signals such as array cutting marker. For example, a higher size element may be placed every 3-10 normal size elements provide visual clues to the user. Either size or its color can be varied in the array. FIG. 8F presents an array resembling 8A but with elements (804) engineered for enhanced swelling capacities. These elements are designed to absorb water upon implantation, expanding to increase size and surface area, as depicted in FIG. 8F1 (805). This feature is beneficial for applications requiring mechanical adaptation post-implantation, such as tissue sealing irregular anatomical spaces or secure anchoring or migration resistance at implantation site. The illustrative example shows only one terminal array element has this ability and is not a limitation of the design. Such beads can be placed at any location in the array and any number of elements can be added to the device depending the requirement of end user. FIG. 8G depicts arrays with drugs embedded in both the thread (806) and the array elements, offering dual pathways for drug release. The drug in array elements and thread can be same or different. Drug concentration and its controlled release characteristics may be same or different. This design is typically achieved by incubating the entire array in a drug solution and coating/infusing thread and array elements as described elsewhere in the specification. In contrast, FIG. 8G1 limits drug loading to the array elements (806), achieved through embedding or threading, leaving the thread (807) uncoated and without drug. These variations provide clinicians with options for precise drug delivery tailored to specific treatment regimens. FIG. 8H shows an array similar to FIG. 8A but with a continuous composition of array elements along the thread length, enhancing uniformity and structural integrity. FIG. 8H2 adds dual terminal threads for more secure fixation. FIG. 8H3 evolves this design further by incorporating multiple threads or matrices and additional anchoring threads (204) at terminal or lateral positions, enabling robust attachment and diverse mechanical properties. FIG. 8I is similar to FIG. 8C but has one, two or more knots (809) are added on the suture to serve as visual or tactile indicators during usage. Some of the beads in the array be used as a sham (no drug) or as a spacer to adjust a desired drug level per unit length of the device. The PLGA beads (210) shown in FIG. 2J can be considered as spacer or sham. The number of sham elements in the device can vary depending the intended use. Number of sham elements in the array may vary from 0 to 100000. The percentage of sham elements may vary from 0 percent to 100 percent, preferably 0 percent to 80 percent and most preferably 0 percent to 50 percent.

A single threaded array device such as shown in FIG. 5D can be configured into a rectangular shape by looping around in a frame (FIG. 7D). This embodiment uses single thread as an illustration, but one, two, three up to 50 or more interconnected arrays can also be used. If desired, the devices may be attached to a rectangular or circular or elliptical, or semicircular or triangular or pentagonal or hexagonal and the like shaped frames (FIG. 7B as an illustrative rectangular frame). The array may be attached to various points to the frame using external thread and knots using desired attachment locations (702, 704 and 705) to preserve the shape/geometry created. Two or more frames along with array device may be stacked to create a desired three-dimensional shape. The density of array elements/beads in such two or three-dimensional frames is preferably 1 array element/bead per centimeter square or more, preferably 10 array elements per centimeter square and even more preferably 50 or more. Total number of array elements in such frames may be 3 or more, preferably 10 or more even more preferably 1000 or more, even more preferably 100000 or more and it can be 1 million to 10 million or higher. The frames may be solid or hollow. For medical devices, frames could be made from biostable or biodegradable materials. Biodegradable polymeric and non-polymeric frame materials including thread used for array attachment are most preferred.

The particles/elements of the surgical array are preferably prevented from migration during storage and use. In some preferred embodiments, this is achieved mechanically by putting knots on both sides of the threaded devices (FIG. 1G, 108; FIG. 2E, 207). Among the different types of knots that can be used in the art, knots that are not easy to unravel are preferred. Knots used in surgical implantation of permanent implants such as vascular grafts or heart valve are preferred.

In one embodiment a rigid tube/disk is placed between the two array elements. FIG. 2J shows a schematic of an array containing a disk-like element (210) like biodegradable hydrogel that is separated by solid rigid hollow cylindrical biodegradable polymer plastic tube/bead like PLGA tube (211). 204 shows the terminal thread portion of the device. The rigid tube 211 helps to lock the position of 210 hydrogel elements in the array and make it migration resistant. The “in situ” casting of particles or disks in the thread or thread junction generally embeds the thread in the crosslinked matrix and therefore makes it resistant to migration. The methods described above are preferred methods to prevent migration/movement of particles/elements/disks of the array.

Other methods like putting an adhesive between the disk and thread can also be used. This invention is not limited to methods as described as above for introducing migration resistance to elements or array. Other methods known in the art or yet to be discovered can also be used and such methods can be used without any limitation.

The present invention addresses the challenges associated with threading hard plastic or crosslinked polymer beads into an implantable surgical array, as depicted in FIG. 1I. Due to the inherent hardness of these beads, threading them onto a flexible thread becomes difficult. To overcome this, the beads are temporarily softened by exposing them to a biocompatible aqueous or organic solvent solution for a sufficient period until sufficient softening that is suitable for threading. Solvent or water acts a softening agent. If needed other agents preferably biocompatible and biodegradable agents such as plasticizers, antioxidants, stabilizers and the like also that can be added during incubation. Other conditions of incubation such as temperature and pressure can also be adjusted. Preferred temperature range is −10 degree C. to 70 degrees C. and preferred pressure is atmospheric pressure. If needed, inter gas atmosphere such as nitrogen or argon gas atmosphere can be used. This softening process makes the beads pliable and suitable for threading without compromising their structural integrity. After threading, the solvent is removed, allowing the bead to substantially restore its original size and shape, as shown in FIG. 1I. This reversible softening process ensures the mechanical properties and functionality of the bead are preserved while facilitating its incorporation into the array. FIG. 1J describes one of the processes for delivering a drug from a hollow bead or disk as an array element. A spherical hollow bead (112), with 113 as its outer casing of desired thickness and 114 as an empty space, is provided. The empty space (114) is filled with a drug delivery composition/visualization agent, either liquid or solid (116). 117 illustrates an opening/hole on bead 115, preferably a pinhole in the 113 casing wall, utilized for the controlled release of the drug or to infuse the drug delivery composition inside the space (114). Alternatively, the particle 115 may be an osmotic pump-based drug delivery device. The size of the hole will depend on the size of the bead.

Preferred diameter of hole may range from 10 microns to 1 mm. This design enables precise and sustained delivery of therapeutic agents, making the beads highly functional in drug delivery systems within the array. In certain medical applications, such as radiation therapy for prostate surgery, it is critical to provide temporary protection to healthy surrounding organs from unintended exposure to radiation. The present invention offers a solution through the use of implantable arrays made from hollow, biodegradable metal beads, such as magnesium alloy beads. These arrays can be strategically deployed to shield healthy tissues while allowing targeted radiation to reach the intended treatment site. The hollow structure of the beads further enhances their functionality by enabling them to be filled with a radio-opaque composition. This filling serves dual purposes: it allows real-time monitoring of the array's coverage and positioning during deployment, and it provides an indication of the beads biodegradability over time. This innovative approach ensures precise and temporary organ protection while maintaining compatibility with the body's natural processes, making it particularly beneficial in sensitive surgical and therapeutic procedures.

The array-like devices disclosed herein can also be made by embedding the thread in a precursor solution prior to crosslinking. FIGS. 3A to 3E demonstrate various methods for creating interconnected particle-based array devices, showcasing a range of techniques and material manipulations in accordance with one or more embodiments. FIG. 3A illustrates a flow diagram of a process to manufacture array devices using liquid compositions and/or precursors utilizing molds. Panel 3A.1 details a mold body (301) with multiple cylindrical cavities (302) and an optional line-shaped groove or cavity for placing a suture or thread (303). Panel 3A.2 shows a thread (304) positioned within the 303 mold cavity. Panel 3A.3 depicts the mold cavities (302) partially or completely filled with different liquid precursor compositions (305 and 306), with the thread (304) partially or completely immersed in these compositions. These precursor compositions are effectively polymerized by exposure to light or other crosslinking mechanisms (307 and 308), securing the thread (304) within the polymerized and crosslinked compositions, as shown in Panel 3A.4. The final polymerized beads/disks with embedded thread (array device) are then removed from the mold, wherein the crosslinked beads/disks (307 and 308) are embedded in the thread, as illustrated in Panel 3A.5. FIG. 3B presents a mold for preparing a sheet/mesh-like array device using a modified version of the method described in FIG. 3A. The mold body (309) features multiple mold cavities (310) arranged in a matrix format (6 columns by 6 rows). Optionally, the mold has a groove to place a thread (311). Threads and precursor compositions are added to the cavities of the mold and then polymerized and crosslinked as described in FIG. 3A.

FIG. 3C displays a matrix-like array device removed from the FIG. 3C mold, showing a 6 by 6 array matrix wherein all beads are interconnected by suture thread and the crosslinked compositions (307 and 308) are located at the intersection of two thread junctions.

FIG. 3D shows a flow diagram for creating an array device using injectable or sprayable liquid precursor compositions. Panel 3D.1 displays a thread, and Panel 3D.2 shows a thread mesh with 25 thread junction points (312). Apparatuses in Panel 3D.3 possess the ability to form fine droplets or spray the desired amount of precursor solution. Panels 3D.31 and 3D.32 illustrate a print head or a syringe with needle (313 and 316), filled with precursor solutions (314 and 317) that can be injected or sprayed via nozzles (315 and 318). The solutions are deposited/sprayed along the thread in a desired sequence (Panel 3D.4) or at the junction point 312 on Panel 3D.2 mesh (Panel 3D.5). The deposited solution is then crosslinked to produce either a single thread array device (Panel 3D.6) or a matrix-type device (Panel 3D.7), wherein the crosslinked compositions (318 and 319) are attached/embedded in the thread or at the intersection of two threads.

Array-like devices also can be made on top of sheet, film, filament or thread. FIG. 4D and FIG. 4E show an array-like device made on top of a sheet. FIGS. 4A to 4E outline methods for creating drug delivery devices using biodegradable threads or films, each method incorporating various drug-loaded compositions, demonstrated using one or more embodiments. FIG. 4A details a method for assembling a drug delivery device wherein several microparticles are connected to biodegradable threads/filaments or strips. Panel 4A.1 displays biodegradable strip/s or filament/s, such as sections of sheep submucosa tissue (401), coated with a precursor composition like gelatin methacrylate solution (402) in water with a photoinitiator (Panel 4A.2). The strip is subsequently cooled to 0 to −10 degrees Celsius to freeze the aqueous precursor composition. Separately, precursor compositions comprising gelatin methacrylate or PEG methacrylate with drug/visualization agents are filled in cylindrical mold cavities and frozen. The frozen precursor compositions (403 and 404) are then placed atop the coated strip 402 in a desired sequence, as depicted in Panel 4A.3. The entire assembly is then exposed to long UV light under frozen conditions to effectively polymerize and crosslink 403, 404, and the coating on the strip (402) to form crosslinked compositions (405 and 406) and a crosslinked coating composition (407), covalently linking the polymerized disks (405 and 406) to the tissue strip via the crosslinked coating 407, forming an array device (Panel 4A.4). FIG. 4B shows an array device similar to that depicted in Panel 4A.4, where disks are replaced with microneedles. FIG. 4C displays an image of the hydrogel array prepared according to one or more embodiments, wherein circular hydrogel disks are arrayed with desired thread length between the disks. FIG. 4D illustrates a drug delivery device where biodegradable spheres or microspheres comprising drug/visualization agents (409) are attached/bonded to the biodegradable prosthetic tissue (410) or biodegradable polymer film/textile via crosslinked coating (407) to create a drug delivery patch. FIG. 4E is similar to FIG. 4H, wherein microspheres are replaced with an array of microneedles (411).

Drug/bioactive compounds or visualization agents can be added to the elements of an array. Various exemplary embodiments suitable for the incorporation of pharmaceutical agents or drugs have been provided. In one preferred embodiment, precursors that produce crosslinked organogels are used to make an array element. Free radically polymerizable precursor like PEG10KL5A, organic solvent (DMSO), free radical initiator, a drug carrier or drug encapsulant (PLGA copolymer endcapped with acetate end groups) and optionally, a drug (bupivacaine base) is mixed to make a homogeneous solution. This homogeneous solution is then added into a mold of desired size and shape and then the precursor in the solution is crosslinked using photopolymerization. The crosslinked photopolymerized disks are extracted from the mold and used to make the array device using a thread as previously described. The drug carrier used in above embodiment (PLGA) preferably does not interfere in crosslinking process and is not covalently bonded to the organogel. Drug carriers like PLGA are physically trapped in the crosslinked organogel. Solvent removal from the disk precipitates the drug carrier (PLGA) within the disk encapsulating the drug (if added) within the precipitated polymer. The controlled release of the drug occurs as the carrier polymer undergoes biodegradation upon implantation in the body. The drug concentration within the bead/disk or array element can range from 0.1 percent to 60 percent, with a preference for 0.5 percent to 40 percent. In the preferred embodiment, the drug concentration is calculated based on the length, area or volume of the device. Drug concentration in the device may be 0.01 percent to 1000 percent of desired drug per centimeter or square centimeter or cubic centimeter of the device, preferably 0.1 percent to 200 percent of the drug. This way, user can adjust the drug dose by choosing appropriate length or area or volume of the device. If a lower dose is needed, then the device can be cut to adjust the drug dose and if more drug is needed, then two or more devices can be joined together such as shown in FIG. 1H to adjust the dose. In addition, the device length can be adjusted or some of the drug loaded elements may be removed or added prior to implantation to adjust the drug dose.

The ability to adjust drug dose during implantation is an important aspect of the device/invention. In the preferred array design, configuration each element of the array is mechanically secured and is migration resistant and therefore device elements will not unravel if the device is cut to adjust the dose. Device features such as terminal thread length and length of thread between each array element is used for cutting and joining devices and adjust its dosing.

The drug is loaded into both the crosslinked material and carrier materials in any proportion. Various polymeric or non-polymeric drug carriers can be used to make organogel/hydrogel based array devices. The drug carriers can be solid, semisolid, low melting solid (melting point below 60 degree C.), neat liquid or gel. Preferred drug carriers can be synthetic polymers or natural polymers. Polymers include homopolymers, copolymers, dendrimers and oligomers or combination thereof. In some applications, natural polymers like proteins with electrostatic/ionic groups that slow drug release via electrostatic/ionic interaction with the carrier polymer are preferred. Biodegradable polymers mentioned in the defined terms part of this application are preferred in controlled drug release applications. Biodegradable polymers that degrade by hydrolysis under conditions found inside the human or animal body (37 degree C. and pH 7.4) are highly preferred. Biodegradable polymers, either synthetic or natural, that degrade by action of natural enzymes (enzymatic degradation) found in the body are also preferred. Synthetic hydrophobic biodegradable polymers are most preferred as drug carriers. Preferred hydrophobic polymers that are made by polymerizing cyclic lactones or carbonates or polymers obtained by polymerizing hydroxy acids are preferred. Preferred synthetic polymers include but not limited to: polymers made from cyclic lactone/carbonate monomers like glycolide, DL-lactide, D-lactide, L-lactide, caprolactone, dioxanone and trimethylene carbonate and the like. Other preferred biodegradable synthetic polymers include but not limited to: polylactic acid; polyglycolic acid; polycaprolactone, polytrimethylene carbonate, polydioxanone, poly(glycerol sebacate), polycarbonate like poly(hexamethylene carbonate), tyrosine-derived polycarbonates, polyarylates and polyethers, PEG-co-polylactone copolymers and the like. Commercially available biodegradable synthetic polymers include but not limited to: poly(dI-lactide-co-glycolide, lactide:glycolide ratio 50:50), poly(dI-lactide-co-glycolide, 65:35), poly(dI-lactide-co-glycolide, 75:25), poly(dI-lactide-co-glycolide, 85:15), poly(dI-lactide-co-caprolactone, 25:75), poly(dI-lactide-co-caprolactone, 80:20) or polymers used in synthetic suture manufacturing can also be used. Preferred drug carriers/polymers have terminal end groups that are capped (endcapped) to improve shelf life and to prevent taking part in precursor crosslinking. The polymer terminal groups include but not limited to: acetate, iodinated compounds, synthetic or natural dyes and the like. The molecular weight of preferred polymers can range from 500 g/mole to 3 million g/mole, preferably 2000 g/mole to 2 million g/mole. Polymers and copolymers obtained by polyethylene glycol-initiated polymerization of cyclic lactone/s or carbonate/s with various degrees of polymerization are also preferred. The degree of cyclic lactone or carbonate polymerization can range from 1 to 300, preferably 2 to 200 depending on PEG molecular weight used. The PEG molecular weight in the copolymers can range from 400 to 35000 g/mole. The PEG used in copolymer formations may have one, two, more hydroxy or other lactone polymerization initiating groups per molecule. The PEG used as an initiator in lactone polymerization may be branched with 2, 3, 4, 5, 6, 7, 8 or more branches. The PEG used as an initiator in lactone polymerization also could be dendrimer. PEG-polylactone polymers that are soluble in water or aqueous buffered/solutions (solubility greater than 1 g/100 g water) are preferred in some applications. The preferred PEG-polylactone polymers are also soluble in alcohols like methanol, ethanol, isopropanol, propanol, PEG or its derivatives, glycerol, butane diol, propane diol or their combinations with each other and water in any proportion and the like.

The PEG-polylactone water/alcohol soluble polymers are especially useful as a carrier for biodegradable hydrogels. The quantity of biodegradable polymer that can be added as a drug carrier can be one percent or higher relative to crosslinked polymer weight. The percent weight of biodegradable polymer carrier in array element varies from 1% to 1000%, with a preference for 5% to 500%, relative to the weight of the crosslinked polymer. The drug can be added in the organogel element prior to polymerization and crosslinking, provided it retains significant biological/therapeutic activity under crosslinking conditions used. Drug incorporation into the organogel can also be achieved after the crosslinking process is completed. The drug can be preferably added via a solvent diffusion process. Organic solvents that have the ability to swell the crosslinked network or organogel at least by five percent or more relative to its original weight/volume and have inability to dissolve the thread are preferred for drug infusion. It is also preferred that the drug (bupivacaine base as an example) and drug carrier used (PLGA) are soluble in the organic solvent used for drug infusion. The solubility for a carrier polymer and constituents of crosslinked gel can be found in chemistry literature databases or in polymer handbook. Alternative it can be also found doing laboratory experiments using standard procedure known in the art. In the illustrative embodiment, the organic solvent used has the ability to swell the organogel by more than ten percent (crosslinked PEG10KL5A) was used.

DMSO is also a solvent for PLGA and the drug bupivacaine base. The disks are incubated in a bupivacaine solution of DMSO for 5 minutes to 24 hours. Variables like pressure, temperature, drug concentration and the like can be altered to make the infusion process more efficient. The amount of drug infused is monitored over a period of time and incubation is stopped when sufficient concentration of drug in the disk/array is achieved. For some compositions, swelling and drug carrier dissolution in the organic solvent takes significant more time, in such situations, it is preferred that the drug infusion takes place in two steps. In the first step, the organogel is incubated in the solvent without the drug. When sufficient swelling and dissolution of the carrier is achieved, then the swollen hydrogel is incubated in a drug solution of organic solvent. Care is taken to ensure that PLGA or PEG-Polylactone (drug carrier if used) is substantially retained in the organogel during the incubation process. It is preferred that at least 40 percent of the drug carrier is retained upon exposure to solvent, preferably greater than 50 percent and most preferably greater than 80 percent drug carrier is retained in the organogel. Those skilled in the art can recognize that the crosslinked density of the crosslinked polymer and drug carrier molecular weight are used as preferred variables to retain the drug carrier inside the organogel during the infusion process. High molecular weight drug carriers/polymers with molecular weight greater than 10000 g/mole preferably greater than 20000 g/mole are preferred if drug infusion process is used. High molecular weight polymers take longer time to diffuse out and therefore are preferred. Crosslinking density can be increased by increasing the number of polymerizable double bonds in the macromonomer and reducing the molecular weight of macromonomer. In some preferred embodiments, non-polymeric carrier and low molecular weight liquid drug carriers like vitamin E, sucrose acetate isobutyrate, and the like can be infused via solvent infusion method in the organogel along with the drug. When desired drug carrier and drug concentration is achieved, solvent is removed leaving behind the liquid carrier and the drug inside the disk. Solvents can be removed through solvent exchange with other solvents, like water or ethanol and the like. Solvent removal by air drying, vacuum drying or lyophilization or their combinations is most preferred. The organic solvent that can be used for organogel preparation or drug infusion include but is not limited to: dimethyl carbonate, methyl ethyl ketone (MEK), tert-butyl acetate, acetone, acetonitrile, cyclohexanone, dimethyl sulfoxide (DMSO), n-methyl pyrrolidinone (NMP), dimethylacetamide (DMAC), dimethyl formamide (DMF), methanol, ethanol, isopropanol, PEG (molecular weight 400-1000 g/mol), PEG endcapped with methyl ether (molecular weight 200 to 1000 Daltons), PEG-based solvents (linear or 3-25 branched), dichloromethane, trichloromethane, chloroform, dioxane, ethyl acetate, dimethyl ether (DME), tripropionin (triprop), tetraglycol, ethyl lactate, triacetin, triethylene glycol dimethyl ether (triglyme), glycerol formal, ethylene glycol monoethyl ether acetate, benzyl alcohol, tributyrin, benzyl benzoate, acetic acid, diethylene glycol dimethyl ether (diglyme), ethyl benzoate, dimethyl isosorbide (DMI), polyethylene glycol dimethyl ether, glycofurol, glycerol, ethyl acetate, 1,3 propanediol, 1,4 butanediol, 1-6-hexanediol, tetrahydrofuran (THF), and the like. Crosslinked polymers derived from macromonomers are presented for illustrative purposes only. Other macromonomers capable of forming crosslinked hydrogels with varying degradation profiles, crosslinking densities, and mechanical properties can also be used. For further details on the synthesis of macromonomers, their biodegradation profiles, crosslinking density, and other pertinent properties, reference is made to the U.S. Pat. No. 6,306,922, herein incorporated by reference for illustrative purposes only. Precursors leading to biodegradable polymers via condensation polymerization can also be used in making organogels or hydrogels. Crosslinked hydrogels synthesized from precursors comprising electrophilic and nucleophilic groups are preferred. In the preferred compositions, total number of electrophilic and nucleophilic groups is greater than five. Crosslinked polymers comprising polyethylene glycol or polyethylene oxide are highly preferred. In one illustrative embodiment, PEG based precursors with 4 amine groups and 4 n-hydroxysuccinimide were used to form crosslinked biodegradable polymers. Total number of nucleophilic and electrophilic groups in this embodiment is 8 (4 nucleophilic+4 electrophilic) which is higher than 5. In some embodiments, precursors made from natural polymers or macromolecules or their derivatives including but not limited to: albumin, collagen, gelatin, fibrinogen, hyaluronic acid, chitosan, hydroxyethyl cellulose, hydroxypropyl cellulose, keratin, silk protein and the like are crosslinked using a crosslinker or its solution. The crosslinker and protein solution are added to the mold just after mixing and before substantial crosslinking or gelation. For example, using a two barrel syringe, gelatin or albumin (10 percent in PBS, pH 7.4) and glutaraldehyde (0.2 percent in PBS) are dispensed in a mold. Both solutions are mixed in the syringe needle before dispensing in the mold. The crosslinking reaction takes place in the mold and crosslinked gel is formed. Similarly hyaluronic acid can be crosslinked using diepoxy or polyepoxy based crosslinker like poly(ethylene glycol) diglycidyl ether. PEG derivatives endcapped with n-Hydroxysuccinimide (NHS) and n-hydroxysulfosuccinimide esters also can be used as protein crosslinkers. Albumin, collagen and other proteins also can be crosslinked using crosslinking catalyzed by 1-ethyl-3-(3-dimethylaminopropyl carbodiimide) hydrochloride (EDC). The EDC crosslinking is generally known as “zero length cross-linking” in the protein modification chemistry art that promotes reaction between carboxylic acid-amine and carboxylic acid-hydroxyl groups in the protein to form amide or ester bond respectively. Water soluble carbodiimides are most preferred. Zero length crosslinkers that can be used in protein crosslinking or to promote between PEG based precursor comprising electrophilic and nucleophilic groups (provided total number of electrophilic and nucleophilic groups is five or more) include but not limited to: 1-(3-dimethylaminopropyl)-3-ethylcarbodiimide methiodide; 1-(3-dimethylaminopropyl)-3-ethylcarbodiimide; 1-cyclohexyl-3-(2-morpholinoethyl) carbodiimide metho-p-toluenesulfonate; 1-(3-dimethylaminopropyl)-3-ethylcarbodiimide and the like. In one embodiment, albumin, EDC and n-hydroxysuccinimide or n-hydroxysulfosuccinimide as a co-catalyst were mixed in a PBS or morpholino ethanesulfonic acid (MES) (pH 5.5 to 6.5) at around zero to 10 degree C. degree C for 5 to 600 minutes to effectively crosslink the albumin in a mold or in situ on a thread as discussed before to form an array element like disk. In some illustrative embodiments, natural polymer solution in organic or aqueous medium like collagen or albumin is frozen first and then frozen solution is exposed to a crosslinker solution like glutaraldehyde solution preferably around freezing point of the solution to crosslink in the frozen state. The preferred natural polymer crosslinkers include but not limited to: di or polyaldehydes like glutaraldehyde, di or polyisocyanate like hexamethylene diisocyanate, di or polyepoxides like 1,4-butanediol diglycidyl ether or poly(ethylene glycol) diglycidyl ether, zero length crosslinker like EDC and the like. Array elements can also be made from natural polymers such a fibrinogen crosslinked or gelled using enzymatic reactions of thrombin and other protein factors found in blood (natural blood clot or fibrin glue components). Other enzyme based protein crosslinking methods known in the art also can be used to make array elements without any limitation. Some synthetic polymers like PEG derivatives can be made with peptide sequence that are recognized and degraded by natural enzymes like collagenease found in the body. Such synthetic polymers are part of enzymatically degradable polymers that can be used as array elements. For additional information regarding crosslinked hydrogels polymerized via condensation polymerization, reference is made to U.S. Pat. Nos. 6,534,591 and 7,009,034, and the references cited therein, all of which are incorporated herein by reference for informational purposes only. In certain embodiments, the biodegradable polymer carrier that exhibits thermosensitive gelation properties is used as a drug carrier in an array element. In one illustrative embodiment, a thermosensitive biodegradable carrier (PEG2KL-1-2) was utilized as a drug carrier. PEG2KL-1-2 exists as a thermosensitive gel at a concentration of 20% and a temperature of approximately 37 degree C. in water or PBS (pH 7.4). Drugs can be entrapped in the thermosensitive polymer during disk preparation or after disk preparation. Other thermosensitive polymers known in the art and include, but not limited to, Pluronic or PEO-PPO copolymers, reverse Pluronic/s or Tetronic/s, polyacrylamides like poly-isopropyl acrylamide and their copolymers or their derivatives, gelatin (various grades), chitosan-based compositions and its derivatives, cellulose derivatives, various PEG-polylactone copolymers, PEG-PLA, PEG-PLGA, PEG-polyhydroxy copolymers, cellulose derivatives and similar compounds, can also be employed. For additional information regarding thermosensitive hydrogels please refer to US Patent Publication numbers US 2019/0046479 and 2022/0118416, and the references cited therein, all of which are incorporated herein by reference for informational purposes only.

In another illustrative embodiment, a neat liquid or low melting solid (with a melting point below 60 degree C.) serves as a drug carrier in the disk. In one specific illustrative example, sucrose acetate isobutyrate (SAB) is utilized as a biodegradable non-polymeric liquid carrier. SAB is mixed with precursor macromonomers in a suitable solvent such as DMSO or ethanol prior to crosslinking optionally with the drug, and the resulting mixture is crosslinked. The SAB remains entrapped within the crosslinked polymer matrix along with the drug. Both polymeric and non-polymeric liquid or low melting carriers can be incorporated into the crosslinked polymers or organogel disk array elements, which include, but are not limited to, biocompatible organic solvents like DMSO, PEG, PEG endcapped with various inert groups, polymeric liquids like low molecular weight polylactone PLGA, or polycaprolactone, ethyl lactate, vitamin E or its derivatives, vitamin E acetate, liquid polylactones or polyhydroxy polymers or copolymers, liquid or low melting PEG-polylactone copolymers, liquid or low melting PEO-PPO-PEO polylactone copolymers, as well as natural or synthetic biodegradable oils and fats, fatty acids, fatty alcohols, oleic acid and its derivatives, and the like. For additional information regarding liquid carriers please refer to US Patent Publication numbers US 2019/0046479 and 2022/0118416, and the references cited therein, all of which are incorporated herein by reference for informational purposes only. While bupivacaine is presented as an illustrative drug in the array, the scope of applicable drugs extends to, but is not limited to: anti-infectives (including antibiotics, antifungal, antiviral, and antibacterial agents), antipruritics, anticancer agents, antipsychotics, cholesterol or lipid-lowering agents, cell cycle inhibitors, antiparknsonism drugs, HMG-CoA reductase inhibitors, anti-restenosis agents, anti-inflammatory agents, antiasthmatic agents, anthelmintics, glucagon-like peptide 1 (GLP-1), immunosuppressives, muscle relaxants, antidiuretics, vasodilators, nitric oxide and nitric oxide-releasing compounds, beta-blockers, hormones, antidepressants, decongestants, calcium channel blockers, growth factors (including bone growth factors and bone morphogenic proteins), wound healing agents, analgesics and analgesic combinations, local anesthetic agents, antihistamines, sedatives, angiogenesis modulators, tranquilizers, and the like. Additionally, therapeutic cellular elements, including but not limited to mammalian cells like stem cells, cellular components or fragments, enzymes, DNA, RNA, mRNA and genes, are also encompassed as bioactive components or drugs. An exhaustive list of potential bioactive compounds or drugs is available in U.S. Pat. No. 8,067,031, herein incorporated by reference for informational purposes only. In some embodiments, drugs can be infused after a desired array device has been made using methods and compositions described in this invention or other methods known in the art or yet to be developed. Preferably such devices have hydrogels and organogels with drug carrier like PLGA entrapped in the array elements. The entire device is incubated in the drug solution until desired drug concentration is achieved in all incubated array elements/beads. The solvent is then removed leaving behind the drug entrapped in the array element and drug carrier if used. It is understood the drug, thread and drug array elements used in the array device can tolerate the infusion conditions as described before. Threads made from gut suture are preferred due to its insolubility in commonly used organic solvents and if organic solvent based compositions are used for drug infusion.

In another illustrative embodiment, drug encapsulated microspheres or microparticles were used as drug carriers. Such drug-encapsulated microspheres are particularly advantageous when employed along with hydrogel-based array elements. In the illustrative embodiment, a biodegradable macromonomer solution (PEG10KL5A in PBS), is mixed with rifampin encapsulated PLGA microspheres along with photoinitiator. This suspension is photopolymerized inside the mold to form into array elements like disks. The disks are then assembled with biodegradable suture material to create an array device. Many macromonomers can be used to make biodegradable hydrogel disks. Crosslinked hydrogels disks derived from condensation polymerization or by free radical mechanism can also be used for encapsulating drug-loaded microspheres. Please refer to the section of organogel preparation methods and related citations for additional information about various precursors to make a variety of biodegradable crosslinked polymer hydrogels. While rifampin is presented as an illustrative drug, the scope of applicable drugs extends to, but is not limited to other drugs mentioned in the previous section. Preferred biodegradable polymers used for preparation of drug encapsulated microspheres are given in defined terms as part of this application. Please refer to preferred polymers in the organogel section for polymers preferably biodegradable polymers that can be used for drug encapsulation. Drug loading within the microspheres can vary from 0.1% to 50%, preferably from 0.2% to 40%, relative to the total microsphere weight. The microsphere concentration within the disk can range from 1% to 80%, preferably 5 to 70 percent relative to the disk's weight. The microsphere size should be under 2 mm, with a preference for sizes between 0.1 microns to 2 mm, and more preferably between 0.2 microns to 1 mm. It is preferred that the microsphere size used is at least 20 percent smaller than that of the disk/bead used in the array device, preferably 80 percent smaller than the disk, more preferably 99 percent smaller, and even more preferably 99.999% smaller or less.

In certain embodiments, the drug present in the array elements or on the array thread may be identical or different, with tailored drug elution profiles tailored to meet specific therapeutic requirements. For instance, when the drug is the same, its release profile can be modified to achieve rapid release (controlled release over 1 hour to 72 hours) or sustained slow release (ranging from 3 days to 1 year). This can be accomplished by varying the formulation of the drug within the thread or array elements, enabling precise control over the timing and duration of drug delivery. Two or more array elements may incorporate the same drug, different drugs, or identical drugs with distinct elution profiles. For example, drug derivatives such as bupivacaine base and bupivacaine hydrochloride, which have differing water solubility and elution profiles, can be used within the array to create a desired release pattern. Additionally, the use of biodegradable polymers or crosslinked polymers as matrices or carriers entrapped in the array element material further enables customization of drug release profiles. Those knowledgeable in the art will recognize that various factors can be manipulated to achieve a specific elution profile, including the type of biodegradable polymer or copolymer and their blends, the molecular weight and distribution of the polymer, its physical state (solid, liquid, or semi-solid) at body temperature, the crosslinking density of the polymer network, and the solvent used during the formulation process. Such parameters or their combinations in any proportions can be precisely controlled to optimize drug release kinetics, providing a highly adaptable platform for diverse medical applications.

Many embodiments use crosslinked hydrogels or organogels to prepare the array device. Crosslinked hydrogels are preferred in many cases but this invention is not limited to crosslinked hydrogels or organogels. One illustrative embodiment depicts preparation of non-crosslinked hydrogels and organogels for preparation of array devices as described in this invention. In one illustrative embodiment, a non-crosslinked gelatin-based hydrogel is used to prepare the array device. A gelatin solution is lyophilized in a mold and the lyophilized gelatin disks are removed then used in array preparation. Preferably such disks are loaded with drug encapsulated microspheres. In another embodiment, non-crosslinked collagen and hyaluronic acid sodium salt is used to make the array device. Yet in another embodiment, a synthetic non-crosslinked hydrogel is used to prepare the array device. Polyvinyl alcohol was used as an illustrative synthetic hydrogel polymer to make the array device. In another embodiment, gelatin based organogel is used. Gelatin was dissolved in organic solvent like DMSO to make a homogeneous solution preferably with a drug carrier such as PLGA. The solution is added in a mold of desired shape and size and the solvent is removed. The gelatin disks with entrapped polymer carriers can be loaded with drug either during the disks preparation or after the preparation using solvent diffusion method. Since many of the hydrogels and organogels preparation do not involve crosslinking, the drug can be preferably added at a desired concentration during their preparation. Gelatin solution in DMSO is added with bupivacaine base as an illustrative drug.

Some embodiments use drug without encapsulation in non-crosslinked hydrogels. Preferably such drugs have low water solubility. Drugs like paclitaxel have low water solubility and can be used without any carrier or encapsulation matrix. The slow dissolution of drug crystals from the array element provides controlled drug delivery profile.

When an array device is used for surgical use, it is preferred that the array device is in solid state, preferably the array elements like hydrogels or organogels are substantially or completely dry.

Preferably array elements have 30 percent less water/solvent, preferably 95 percent less water/solvent and most preferably 99 percent less water/solvent relative to its fully hydrated/solvated state. Some dehydrated organogels or hydrogels, especially those that undergo biodegradation via hydrolysis process, have better shelf life in dry state. Upon implantation of the device, the array elements absorb water present at the implantation site like tissue fluids, blood or other biological fluids and become fully hydrated. The array elements of the device can absorb 5 percent or more preferably 10 percent of more water relative to its original dry weight or volume upon implantation. In some embodiments, organogels and hydrogels of the array device can be completely hydrated or solvated. Array elements that degrade by enzymatic pathways like collagen, gelatin or hyaluronic acid and array threads made from gut suture can be stored in hydrated form. Some organogels and hydrogels array elements can be hydrated/solvated with water soluble biocompatible organic solvents like DMSO, polyethylene glycol or its derivatives, n-methyl pyrrolidinone, ethanol, glycerol and the like. When implanted, organic water-soluble solvents are displaced by the tissue fluid and are safely removed by the body.

To improve the implantation of the array device, the thread and array elements of the device are intentionally colored. The color helps to improve visibility of the device and therefore helps the device to implant at a desired location. Any biocompatible color can be used. Colors that are approved by the government regulatory agencies for pharmaceutical products or for implantable biostable or biodegradable medical devices are preferred. Colors that are safely removed after implantation are most preferred. Preferred list of colored compounds is provided in the definition section and such compounds and their combinations may be used in any proportion. It is preferred that color of the thread is different than the color of array elements. Preferred colors are blue, green, yellow, orange, red or their dark or light and other shades. If an array element has a two or more types of drugs, then such elements/beads may be differentiated by color. For illustrative purposes, if an array element contains bupivacaine as a drug, it will be intentionally colored blue. Conversely, if an array element contains an antibiotic as a drug, it will be intentionally colored red. If both drugs are present in the same array device as separate beads or elements, their presence and location within the device will be visually differentiated or verified using red and blue colors. It is preferred that the color intensity will reflect the drug concentration in the array element. Lower drug concentration is preferred to have lighter shades of color and higher drug concentration have darker shades of color or vice versa.

The array device disclosed in this invention can have array elements that are porous in nature. The term “porosity” is defined as the presence of pores, voids, cavities, grooves, pockets and indentations within a material or on its surface or both. The porous structure of beads can be used in many ways. It can be used to enhance ultrasonic visibility or it can be used to load drug or visualization agents. Artificial porosity in the array particle may be created in many ways as known in the polymer foam preparation art. The porosity preparation methods include but not limited to: creating a gas source inside the array particle matrix such as carbon dioxide gas by decomposition of sodium bicarbonate; removal of solvents by freeze drying; adding a porogen such as water soluble salt and leaching it out from the solid matrix and the like.

Porosity can be incorporated many methods known in the tissue engineering scaffold preparation or polymer foam preparation art. Non-limiting illustrative embodiments are provided an Example 8. Two widely used techniques to induce porosity are salt leaching and lyophilization. In one embodiment, a finely ground sodium chloride is mixed with a polymer solution, frozen, and lyophilized. Subsequent incubation in water dissolves the salt, leaving behind voids that form the pores. By varying the salt particle size and concentration, the pore size and porosity can be precisely controlled. This method is exemplified by porous structures made from Poly(L-lactide-co-caprolactone). Another approach involves leaching organic soluble polymers. Polymer microspheres, such as poly(ethyl methacrylate), are embedded in a matrix and extracted with a solvent, creating spherical voids. Many types of polymeric beads in various sizes are available commercially and such beads may be used to induce porosity. This method is demonstrated with collagen and Poly(L-lactide) matrices, producing spherical pores that can be used for drug delivery or other applications. Similarly, mechanical or laser drilling introduces porosity by creating small holes, grooves, cavities on polymer rods or molded sections of biostable or biodegradable polymers. This technique offers surface porosity customization for biodegradable plastics materials like polylactic acid and polycaprolactone. For hydrogels, porosity is created by mixing crosslinkable macromonomers (e.g., PEG derivatives) with porogen like sodium chloride or dissolvable microspheres. After crosslinking and porogen removal, the hydrogels exhibit interconnected porous networks. Crosslinking can occur via photo-initiators or electrophilic and nucleophilic interactions, as demonstrated with PEG-based hydrogels. Another method involves acid removal of carbonate salts embedded in protein solutions like albumin. The salts are dissolved in mild acidic solutions, leaving behind porous protein microspheres. Many hydrogels have 30-90 percent water in the structure that is removed by lyophilization process and porosity thus created is used for drug loading and other applications. The size or average diameter of porogen must be less than the array element size, preferably less than 80 percent, even more preferably less than 50 percent and even more preferably less than 10 percent. The preferred materials include materials that have well defined particle size and distribution which may include but not limited to: commonly used salts and compounds such as, sodium bicarbonate, calcium carbonate, sodium chloride, potassium chloride, sugar, glucose, PEG of various molecular weights and the like. In choosing the porogen, it is essential that it can be extracted from array element without affecting the size, structure and shape of the array element. Many inorganic and organic water/organic solvent soluble salts are commercially available with size ranging from nanometer to millimeters and such salts also can be used without limitation to induce porosity. Average size of pores may be higher than 100 nm, and range from 100 nm to 2 mm, preferably 0.5 microns to 1 mm. The porosity of particle or array element may range from 5 percent to 95 percent, preferably 10 to 90 percent. Many hydrogels have 30-90 percent free water in the structure. The water may be removed by lyophilization to create porosity. The preferred porous structure is interconnected pores where pores can be filled with drug delivery compositions. The porous hydrogel array elements may be made from natural polymers like gelatin, collagen, chitosan, hyaluronic acid, alginate and the like. The porous biodegradable hydrogel microparticles may be made from synthetic polymers such as polyvinyl alcohol, polyvinyl pyrrolidinone, polyamino acids, polypeptides, PEG based hydrogels, PEG-polyhydroxy acid copolymers or PEG-polytrimethylene carbonate based copolymers and the like. The porous hydrogel elements in the array used may be crosslinked or not crosslinked. Crosslinked hydrogels are preferred because they can be insoluble in many organic solvents, which can help to maintain its physical integrity in porosity creation and drug infusion step. PEG based crosslinked porous hydrogels may be obtained by crosslinking of water soluble macromonomers (PEG-lactone based macromonomers) or condensation polymerization of PEG based precursors/crosslinkers and other compounds are preferred. PEG based crosslinked hydrogels can be obtained with various degradation times ranging from few days to few years depending on the hydrolizable component used in the PEG composition. Standard porous synthetic biodegradable plastics such polylactide, polylactide-polyglycolide copolymers, polycaprolactone and the like can be made porous using techniques described earlier. Such plastics may be first cast or injection molded or extruded or 3-D printed and porosity is created using laser drilling or mechanical drilling disclosed in this invention. For threading purposes, it is preferred that such materials are soft and can be easily threaded. For hard materials, holes in the materials must be created and used for incorporating in the array.

The porosity created may be used to fill drug delivery compositions for sustained drug delivery. In one illustrative embodiment. The drug loaded particles can be cast in a desired shape. Alternatively porous particles are prepared first and drug may be infused in the pores of the particles. A biodegradable carrier polymer may be used along with the drug to control the release rate from the device. If drug is infused without the carrier, the drug is preferred to have low water or physiological fluids solubility. Drug solubility is generally is less than 1 g/100 g of water, preferably 0.5 g/100 g of water. Many synthetic or natural biodegradable polymers and non-polymers can be used a carrier. The list of preferred biodegradable polymers is mentioned in definition section of this invention. PEG-polylactone and polylactones/polyesters are most preferred carriers. The drug carrier in the pores may be solid, semisolid, wax, liquid, thermosensitive or pH sensitive gel. Preferred liquid and thermosensitive gels are discussed in earlier section. Preferred drug concentration, array particle size and shape have discussed in earlier section of the document.

In certain embodiments, the array device is designed to incorporate visual or tactile indicators or cues to assist in its handling, placement, and customization during surgical procedures or therapeutic use. These visual or tactile indicators serve as intuitive guides for users, providing specific instructions or markers include but not limited to such as “cut here for a particular drug dose” or “form a loop at this point.” Such indicators can be integrated into both the array elements and the connecting threads. Examples of visual cues include but not limited to varying thread lengths, as depicted in FIG. 8C, where distinct thread lengths signify different actions or configurations; bead size variations, as shown in FIG. 8E, where a bead (803) with a larger size relative to neighboring beads acts as a marker; or the presence of knots, as illustrated in FIG. 8I, where double knots provide a visual and tactile indication for a specific action. Additional visual or tactile cues may involve differences in bead color, shape, or spacing, which are not limited to the examples provided but can encompass any modifications detectable by the unaided human eye or by human touch (knots and number of knots can be assessed by touch, an example of tactile non-visual clue). These visual indicators enhance the usability and precision of the array device, enabling healthcare professionals to customize and implement the device effectively for favorable/tailored therapeutic outcomes and ease of use during surgical procedure.

The array devices described in this invention can be made sterile by using sterile techniques known in the art. The entire array preparation operation is performed in a sterile manner wherein the thread and precursors are pre-sterilized before using. Subsequent operations like in situ casting and lyophilization can be done in a sterile clean room environment. The array device, preferably with lyophilized or dry or dehydrated array elements can be post sterilized and packaged using methods known in the art but not limited to like: ethylene oxide sterilization, hydrogen peroxide sterilization, radiation or electron beam sterilization and the like. The array elements in the array device as may have different size, shapes, drug release profiles and the like. Following array element properties are preferred: at least one of the array element has drug/visualization agent; at least one of the array element that degrades by enzymatic degradation; at least one of the array element that is substantially dehydrated; at least one of the array element has encapsulated live cell, bacteria, active synthetic or natural enzyme or catalyst; at least one of the array element is made from crosslinked polymer; at least one of the array element is porous; at least one of the array element has capacity to absorb 10 percent of more water relative to its weight at the time of implantation; at least one of the array element has different size; at least one of the array element has different shape; at least one of the array element has different thread length between two array elements (x1); at least one of the array element is hydrogel, at least one of the array element is organogel, at least one of the array element is biostable polymer or plastic, at least one of array element is metal, at least one of array element is glass, at least one of the array element has liquid drug carrier, at least one of the array element is thermosensitive or pH sensitive gel as drug carrier; at least one of the array element has synthetic biodegradable polymer carrier, at least one of the array element has crosslinked polymer that is produced by precursor comprising electrophilic and nucleophilic groups; at least one of the array element has crosslinked polymer that is produced by free radical polymerization mechanism. The combinations of above element types in any proportion are also preferred.

If an array has two or more elements with different physical or chemical properties, then following combinations of physical or chemical properties are preferred without any limitations: at least one of the array element has different drug than the other elements; at least one of the array elements has different drug concentration than the other elements; at least one of the array elements releases the drug at least five percent faster or slower rate than the other elements; at least one of the array element has different crosslinking density than the other elements; at least one of the array elements has different biodegradation mechanism than the other elements; at least one of the array elements has different size than the other elements; at least one of the array elements has different color than the other elements; at least one of the array elements has different shape than the other elements; at least one of the array elements has different length of thread between two elements (x1) than the other elements; at least one of the array elements has different water (10 percent or more) absorption capacity than the other elements; at least one of the array elements has different radio-opaque property than the other elements; at least one of the array elements has different density than the other elements; at least one of the array elements has different drug carrier than the other elements; at least one of the array elements has liquid drug carrier than the other elements; at least one of the array elements has thermosensitive or pH sensitive polymer as drug carrier than the other elements; at least one of the array elements has synthetic biodegradable polymer as a drug carrier than the other elements; at least one of the array elements is made from crosslinked polymer; at least one of the array elements is made from non-crosslinked polymer; at least one of the array element is cast from a liquid/solution of a precursor or polymer/macromolecule, at least one of the array element has a drug carrier and the drug carrier may be solid, liquid, or gel.

Ability to retrieve a deployed drug delivery is an important aspect of this invention. Since drug loaded microparticles are connected via thread, it offers ability to retrieve some or part of the device to be retrieved or removed from the surgical site. FIG. 10B illustrates a minimally invasive surgical (MIS) device equipped with operational components (204) designed to perform various surgical tasks. These components may include instruments for deploying MIS tools, integrated cameras, or cleaning fluid delivery lines. The array device is stored in a spool (1005) and deployed through a trocar or tube (1004) via a pulley mechanism (1006) that enables precise control over the movement of the array. The spool and pulley system facilitates forward and backward motion, allowing the array to be extended or retracted as needed during the procedure. The array device is specifically designed to fit within the dimensions of the trocar (1004), ensuring seamless deployment and retraction. This compact and efficient design enables surgeons to introduce and manage functional elements with high precision, enhancing the versatility and utility of the MIS device in various surgical applications.

In some embodiments, the implantable array device is integrated with various sensors, preferably electronic sensors, to enhance its functionality for medical, industrial, or research applications. The array may include a power supply, such as an electrical battery, preferably positioned at the terminal end or in the middle section. In such embodiments, the interconnecting thread serves a dual purpose: providing structural support for the array and acting as an electrical conductor to transmit signals between the sensors and the power source or external monitoring equipment. The array device may incorporate sensors to monitor physiological parameters for medical applications. These sensors can include, but are not limited to, blood oxygen level sensors, heart rate monitors, electrocardiogram sensors, blood pressure sensors, insulin level detectors, blood alcohol concentration sensors, and other diagnostic tools. Such a configuration enables real-time monitoring and data collection within the body, supporting advanced therapeutic interventions and patient management. For arrays used in bioreactors or other industrial processes, the integrated sensors can measure critical process parameters, including temperature, pressure, pH, solvent concentration, product concentration, light absorbance for turbidity analysis, oxygen concentration, water concentration, and more. These sensors facilitate precise monitoring and control of the bioreactor environment, optimizing conditions for cell growth, enzymatic reactions, or chemical production. The incorporation of sensors within the array provides a highly versatile platform for diverse applications, enabling the measurement, monitoring, and response to real-time alterations in the surrounding environment. Such sensor-integrated designs are not confined to the specific embodiments described herein but encompass all configurations that effectively achieve the intended diagnostic or process control functionality.

In certain embodiments, one or more array elements possess magnetic properties, enhancing the device's functionality in both medical and industrial applications. The inclusion of magnetic elements facilitates the retrieval of the device post-implantation using external magnetic tools, thereby improving the device's safety and practicality. In one embodiment, a macromonomer solution or suspension is prepared comprising a photoinitiator and magnetic ferrite powder or iron oxide as a magnetic filler. The solution is filled into a cylindrical mold cavity and exposed to ultraviolet light to initiate polymerization and crosslinking. After approximately five minutes of exposure, the crosslinked gel disks with magnetic particles entrapped within the gel are removed and tested for magnetism using a laboratory magnet. Subsequently, the magnetic gels are utilized to construct an array device as previously discussed. For applications in bioreactors, the magnetic property enables efficient separation of the array from the reaction medium, simplifying downstream processing and device reuse.

For devices incorporating soft hydrogel-like elements, there exists a potential risk that the interconnecting thread may function as a cutting tool, potentially compromising the integrity of the elements during the manufacturing, transportation, storage, or user handling process. To mitigate this risk, several approaches are disclosed. A preferred method involves applying a coating, preferably a biodegradable coating, to the thread. This coating increases the thread's diameter, thereby reducing its cutting propensity and enhancing the overall structural integrity of the device. An alternative approach involves designing specialized packaging to immobilize the device during storage and transportation. By restricting the movement of the thread and elements within the package, the likelihood of damage is significantly minimized. In another preferred embodiment, the softness of the hydrogel-like elements is reduced by substantially or completely removing solvent or water from the elements. This is achieved through lyophilization or drying, resulting in elements that are structurally more stable and less susceptible to mechanical damage.

Furthermore, additional methods known in the art or developed in the future for addressing the cutting action of the thread are also contemplated as part of this invention. These approaches collectively ensure the integrity and functionality of the device are maintained throughout its entire lifecycle.

### Medical Applications of Arrays

### Application as a Surgical or Biopsy Marker

The array devices disclosed herein facilitate the encoding and decoding of information by leveraging the composition, arrangement, and sequence of array elements to generate a unique identification code. This code enables precise device identification post-implantation, particularly for surgical markers such as non-magnetic breast biopsy markers. These markers are identifiable by their distinctive shapes and imaging signatures in ultrasonic or X-ray images. The inventive arrays utilize the sequence and positioning of elements, along with their physical or chemical properties, to produce numeric or alphanumeric codes for identification. FIG. 9 illustrates the schematic representation of these arrays. FIG. 9A outlines the process of deriving a code from an array. The array contains elements with distinct properties, such as X-ray absorption, fluorescence, or shape. These properties are measured through imaging techniques like X-ray or infrared/visible light cameras, ultrasonic imaging and the like with differences assigned alphanumeric values (e.g., 0 or 1). By altering the sequence of elements and repeating the measurements, unique numeric codes are generated. These codes serve to uniquely identify the implanted devices, such as biopsy markers. An example array in FIG. 9B comprises gold (901) and titanium alloy (Nitinol) beads (902) arranged in a specific sequence. Upon X-ray imaging, the gold bead (901) appears brighter due to its higher density and X-ray absorption compared to Nitinol. This difference assigns 0 to the gold bead and 1 to the Nitinol bead, generating a code of 0101 for configuration B1. Rearranging the sequence in B2 results in a code of 1001. FIG. 9C1 shows an array with spherical (903) and cross-shaped (904) Nitinol elements. X-ray imaging assigns codes based on shape, yielding 1010, while rearranging the sequence in C2 results in 1001. The elements are designed with smooth shapes to minimize tissue irritation or injury during physical activity. FIG. 9D depicts an interconnected disk-shaped array without threads, where elements (905-908) have distinct properties. These elements are encased in a biocompatible outer casing (909) made from materials such as high-density polyethylene, polytetrafluoroethylene, polyethylene terephthalate, or polymethyl methacrylate, ensuring long-term implantation safety. These materials, preferably approved by regulatory agencies, do not interfere with imaging, allowing property-based identification. Encoded measurements from these properties yield values such as 0, 1, 2, or 3. The arrays illustrated in FIG. 9B, FIG. 9C, and FIG. 9D are examples, with preferred configurations comprising two or more, preferably three or more, and most preferably six or more imaging elements. Suitable 3D shapes include spheres, cubes, and cylinders, which may be symmetrical, asymmetrical, or random. The array elements are configured into compact shape so that they can be easily deployed using MIS techniques and re-expanded “in situ” if needed. Non-magnetic metals or alloys, such as gold, silver, titanium, and their derivatives and the like, are preferred materials. Additional features, such as adjustable distances between elements and non-migration characteristics, are also disclosed, making these arrays versatile and highly functional for clinical applications.

### Application in Surgical and Non-Surgical Wound Management

The drug-loaded array device can be effectively used in managing surgical or non-surgical wound sites. The drug loaded array infused with anti-infective agent is placed and sutured at the site. Terminal threads or thread between array elements is used to attach the device at the wound site. Preferably the device is placed and attached at junction wound closure wherein probability of infection is higher. This attachment assures local drug delivery at the device attachment site.

The user can adjust drug dose by cutting a section of the device or extending the length by two or more devices as described in this invention. Other drugs that may be delivered via array device include anti-scarring agents to minimize scar formation, antibiotics or germicides to prevent local infections, and local anesthetics for pain relief lasting 1-5 days. The beads within the array can be designed to contain multiple medications in a single device or separate arrays can be used for each specific medication. This modular and customizable approach ensures precise and efficient delivery of therapeutic agents tailored to the needs of the wound site, promoting faster healing and improved patient outcomes.

### Veterinary Applications

For veterinary applications, particularly for large farm animals such as horses, cows, and pigs, the array device is meticulously designed with enhanced dimensions and structural attributes to accommodate their larger anatomical features. The array elements, comprising beads or microparticles, are notably larger in size, ranging from several millimeters to a few centimeters in diameter. This increased size facilitates a higher drug-loading capacity, enabling the delivery of therapeutic agents in quantities proportionate to the body mass of these animals. The interconnecting threads are constructed from durable, biocompatible materials with enhanced thickness and tensile strength to withstand the mechanical stresses of deployment, handling, and in vivo movement. The thread lengths between array elements can be customized to ensure optimal spacing and secure anchorage at target sites. The beads are engineered with tunable porosity, adjustable between 2% and 90%, to regulate the release kinetics of bioactive agents and to accommodate the dynamic tissue fluid environment of larger animals. The array device is designed for minimally invasive deployment, with the capability to compactly fold for insertion and expand to its functional form post-deployment. To ensure retrievability, the device incorporates extended threading and reinforced attachment points. Furthermore, visual markers, such as color coding or physical patterns, are integrated to indicate drug types, concentrations, or placement zones, facilitating ease of identification and customization during surgical or therapeutic procedures. This modular design enables the creation of interconnected segments that can be scaled and adapted for various species or specific anatomical regions. The device supports single or multi-drug configurations to address a comprehensive range of veterinary requirements, including but not limited to anti-inflammatory, antibiotic, or wound-healing therapies and the like. These multifaceted features render the disclosed array device an advanced solution for localized and sustained drug delivery in veterinary medicine, offering enhanced therapeutic outcomes and adaptability for diverse clinical scenarios.

### Spiral Application on Vascular Grafts or Stent Grafts

The array device can be adapted for use in vascular graft surgeries or stent graft surgeries or other surgeries involving tubular organs such as arteries, veins, small or large intestine and the like, particularly in peripheral or small-diameter synthetic graft or natural tissue graft procedures. The device is placed at the junction of the grafting site and sutured in position. The device can be looped around or spirally attached multiple times if needed to adjust the drug dose. It can be loaded with one or more anti-restenosis agents, such as paclitaxel, rapamycin, everolimus zotarolimus, biolimus A9 and the like. These agents are released locally at the graft site, providing targeted therapy to reduce the risk of restenosis. This localized drug delivery is expected to enhance graft longevity by mitigating restenosis-related failures, which are a common complication in vascular graft surgery.

In another application, the array device is designed to be spirally wound around synthetic or natural vascular grafts, particularly expanded PTFE (ePTFE) small or medium-sized grafts or stent grafts. This configuration enhances the mechanical and therapeutic properties of the graft. The thread component of the array provides anti-kinking properties, ensuring structural integrity and proper blood flow, while the beads deliver localized anti-restenosis agents, such as paclitaxel or rapamycin. This dual functionality not only expected to prevent restenosis through localized drug therapy but also improves the graft's durability and functionality during and after the surgical procedure. Due to high flexibility of array device, the drug loaded array device can also be mounted or attached to biodegradable or biostable stents or balloons. Coronary, peripheral and other stents and stent grafts can be fitted or modified with drug loaded arrays. This way drug coating on the stent itself may be avoided. Stents with drug loaded array elements can serve as an alternative to drug coated stents.

### Application in Dental Care for Gum Disease Management

The array device can also be adapted for use in dental applications, specifically for the management and treatment of gum diseases. The device can be positioned in the interdental spaces or along the gum line, filling the space between the gum and teeth. By delivering localized therapeutic agents, such as antibiotics, anti-inflammatory drugs, or germicides, the device targets the bacterial infections and inflammation associated with gum diseases. The beads within the array can be designed to release these agents gradually over time, ensuring sustained and effective treatment. This configuration not only addresses active gum disease but also aids in maintaining oral hygiene and preventing disease progression. The flexible and modular nature of the array allows it to conform to the unique anatomy of the gum and tooth interface, making it a valuable tool for personalized dental care.

### Application in Reducing Surgical Adhesions

The array device can be particularly effective in reducing the formation of surgical adhesions, a common complication following surgeries. Its high flexibility, biodegradability, and ability to be securely anchored make it an ideal solution for placement in areas prone to adhesion formation, such as abdominal, pelvic, or cardiovascular surgical sites. The device can act as a physical barrier, preventing tissues from adhering to one another during the healing process. Additionally, the array can be loaded with therapeutic agents, such as anti-inflammatory drugs or anti-fibrotic agents, to actively inhibit the biological processes that lead to adhesion formation. Its biodegradability ensures that the device naturally dissolves once it has fulfilled its function, eliminating the need for a second procedure for removal. By combining physical and pharmacological strategies, the array device provides a multifaceted approach to preventing surgical adhesions, improving post-operative outcomes, and enhancing patient recovery. Its adaptability and ease of placement further underscore its utility in managing this challenging surgical complication.

Applications in Production of Industrial Chemicals and Therapeutic Agents by Encapsulated Active Enzymes, Live Bacteria or Mammalian Cells.

The incorporation of enzymes within hydrogel matrices in array devices presents significant advantages for industrial chemical production. The industrial enzymes that can be utilized in arrays include, but are not limited to, lipase, trypsin, amylase, glucose isomerase, papain, pectinase, protease, cellulase, xylanase, nitrile hydratase, transaminase, monoamine oxidase, and penicillin acylase, among others. One or more enzymes per array can be employed to achieve a desired industrial chemical. The interconnected array configuration enables concurrent or sequential catalytic reactions, thereby enhancing process efficiency and product selectivity. The biostable hydrogel environment sustains enzyme stability and activity by providing a hydrated, biocompatible setting that maintains the natural structure of the encapsulated enzymes. Biostable hydrogels utilized in array preparation can be specifically tailored for a particular industrial process. For instance, porosity of the hydrogel, chemical composition, molecular permeability, array spacing, array element density, bead size, array length and the like can be employed to customize the hydrogel for a specific industrial process. Crosslinked hydrogels are particularly favored in this application. Array properties, such as the size of the array element, bead arrangement, the number of array elements, the number of arrays used, and the spacing between arrays, can be tailored to suit a particular industrial process. Furthermore, immobilization simplifies enzyme recovery and reuse, thereby reducing operational expenses and minimizing downstream processing challenges. Implementing these hydrogel-based enzyme arrays in industrial contexts, such as food processing, pharmaceuticals, paper manufacturing, detergents, and biofuel production, enables the creation of compact, efficient, and scalable bioreactors. For instance, in food production, immobilized amylase and glucose isomerase can be combined to transform starch into high-fructose syrups. In biofuel manufacturing, cellulase and xylanase arrays can facilitate the breakdown of lignocellulosic biomass into fermentable sugars. The modular nature of array devices facilitates the customization of enzymatic pathways tailored to specific production requirements, thereby enhancing flexibility and adaptability across various industrial applications. Additionally, the utilization of hydrogels as immobilization matrices offers advantages such as biocompatibility, adjustable porosity, and ease of functionalization, further optimizing the performance of the encapsulated enzymes. Bacteria, particularly those belonging to the Streptomyces genus, are pivotal sources of antibiotics and other bioactive compounds. Prominent examples include Streptomyces griseus, which produces streptomycin, an effective antibiotic for treating tuberculosis; Streptomyces aureofaciens, known for producing tetracycline, a broad-spectrum antibiotic; and Streptomyces venezuelae, which generates chloramphenicol, used to combat severe infections. Antifungal medications such as nystatin and amphotericin B are derived from Streptomyces noursei and Streptomyces nodosus, respectively. Anticancer agents like doxorubicin and bleomycin, derived from Streptomyces peucetius and Streptomyces verticillus, respectively, play a crucial role in chemotherapy protocols. Furthermore, immunosuppressants such as sirolimus (from Streptomyces hygroscopicus) and tacrolimus (from Streptomyces tsukubaensis) are essential in preventing organ rejection in transplant recipients. Streptomyces avermitilis has produced ivermectin, a widely utilized antiparasitic drug. These bacterial products have revolutionized modern medicine, providing treatments for infections, cancer, and other intricate disorders. Bacteria utilized in pharmaceutical product manufacturing can be encapsulated within the array devices disclosed herein. The hydrogel-based arrays disclosed herein can be tailored to encapsulate live bacterial cells, and the array properties can be customized for specific bacteria.

Mammalian cell cultures have become indispensable in the biopharmaceutical industry, particularly for the production of monoclonal antibodies, hormones, and therapeutic proteins. Chinese hamster ovary (CHO) cells, murine myeloma cells, and other mammalian systems facilitate the production of biologics that require intricate post-translational modifications. Monoclonal antibodies such as adalimumab (for autoimmune conditions), trastuzumab (for HER2-positive breast cancer), and rituximab (for lymphomas) are generated in mammalian cells. Fusion proteins like etanercept, used in managing autoimmune diseases, and aflibercept, a treatment for macular degeneration, are also derived from mammalian cells. Hormonal drugs such as erythropoietin, which stimulates red blood cell production, and follicle-stimulating hormone (FSH), which aids in fertility treatments, owe their availability to this technology.

Clotting factors like Factor VIII (for hemophilia A) and enzymes such as idursulfase (for Hunter syndrome) are similarly produced. These biopharmaceuticals represent significant advancements in treating chronic and life-threatening conditions, underscoring the crucial role of mammalian cell cultures in improving healthcare. The hydrogels arrays disclosed in this invention can be utilized to encapsulate live mammalian cells, such as CHO cells, murine myeloma cells, and other mammalian cells. Various array configurations and array element properties can be tailored to optimize a specific process, thereby achieving highly efficient biologic drug production.

### Application in Clinical Diagnostics

Hydrogel-based arrays provide a flexible platform for clinical diagnostics, allowing for concurrent detection and measurement of multiple biomarkers in a single test. Each hydrogel element in the array can secure a specific antibody or analytical agent, enabling multiplex analysis of protein expression, function, or interactions. This feature aids in identifying disease-specific biomarkers, evaluating patient outcomes, and tracking treatment responses, making these arrays highly beneficial in clinical environments. The three-dimensional, water-loving nature of biostable hydrogels creates an environment similar to a solution, which enhances the stability and activity of immobilized biomolecules, thereby improving detection sensitivity and specificity. The adjustable porosity of hydrogels facilitates efficient diffusion of target analytes, optimizing diagnostic assay performance. This characteristic makes hydrogel-based arrays particularly well-suited for analyzing complex biological samples, such as blood, serum, plasma, urine, sweat, saliva and tissue lysates and the like, with high precision and consistency. By incorporating various antibodies or analytical agents into individual hydrogel elements within a single array, comprehensive profiling of multiple biomarkers associated with different diseases becomes possible. This approach streamlines diagnostics, conserves sample material, and reduces testing time. The capacity to perform multiplexed diagnostics on a single array supports applications ranging from early disease detection to personalized medicine strategies, highlighting the significant potential of hydrogel-based arrays in advancing clinical diagnostics.

### Application of the Array in Radiation Protection

The array device presents substantial potential for radiation protection applications, particularly in procedures such as prostate surgery, where shielding surrounding healthy organs from radiation is paramount. The array, composed of hollow or solid biodegradable hydrogel or metal beads, such as magnesium alloys beads, can be deployed to construct a protective barrier around sensitive tissues. Its compact and continuous design enables it to fill large spaces uniformly, ensuring comprehensive coverage and effective radiation shielding. For deployment, the array can be stored on a spool outside the body and delivered via a minimally invasive surgical (MIS) device (FIG. 10B). FIG. 10B illustrates a minimally invasive surgical (MIS) device equipped with operational components designed to perform various surgical tasks. These components may include instruments for deploying MIS tools, integrated cameras, or cleaning fluid delivery lines.

The array device is stored in a spool (1005) and deployed through a trocar or tube (1004) via a pulley mechanism (1006) that enables precise control over the movement of the array. The spool and pulley system facilitates forward and backward motion at the rate of 0.1 mm per minute or more, preferably at 0.1 mm to 500 mm per minute, allowing the array to be extended or retracted as needed during the procedure. The array device is specifically designed to fit within the dimensions of the trocar (1004), ensuring seamless deployment and retraction, highlighting its utility in surgical interventions and patient care. This method facilitates precise placement into target cavities with minimal patient discomfort. The spool-based design facilitates controlled deployment, and the flexible nature of the array ensures it conforms to irregular shapes for seamless coverage. If necessary, the array can be adjusted by cutting or joining segments without the risk of unraveling, providing surgeons with the flexibility to tailor its size and configuration to the specific therapeutic requirements. Furthermore, the hollow beads can be filled with a radio-opaque composition, enabling real-time monitoring of the array's position, coverage, and biodegradability during and after deployment. This feature enhances the device's functionality and ensures its effectiveness as a temporary radiation shield that biodegrades safely after fulfilling its purpose.

### Ophthalmic Applications of Arrays

The arrays disclosed in this invention demonstrate substantial potential for enhancing ophthalmic applications, offering innovative solutions for both therapeutic and diagnostic purposes. These arrays can be tailored for incorporation into devices such as punctal implants, contact lenses, and ophthalmic drug delivery systems, addressing critical needs in ophthalmic care. One notable application of the arrays is in punctal implants, where the design is optimized for localized drug delivery and enhanced anchoring. Arrays, as illustrated in FIG. 8F, incorporate elements (804) engineered for increased swelling capacities. Upon implantation, these elements absorb water and expand, as shown in FIG. 8F1 (805), which increases their size and surface area. This swelling capability is particularly advantageous for punctal implants, where the terminal array elements can swell more than the others to secure the implant in place while maintaining functionality. Additionally, some array elements can be intentionally colored or fluorescent to facilitate visibility and monitoring after implantation. For example, fluorescein or similar ophthalmic dyes can be covalently linked or encapsulated in the array, ensuring safe, biocompatible, and traceable devices. These implants, typically measuring 0.2-0.8 mm in diameter and 1-2 mm in height, can also serve as localized drug delivery systems. Drug-loaded arrays, such as those carrying dexamethasone, enable sustained release of medication directly at the site, minimizing systemic exposure and improving therapeutic outcomes for conditions like inflammation and dry eye. The invention also expands the functionality of contact lenses through the integration of arrays into their design. Arrays embedded in contact lenses are crafted using biostable and transparent hydrogel materials to ensure durability, biocompatibility, and optical clarity. These hydrogels resist degradation in aqueous environments, making them suitable for prolonged use and storage in typical lens solutions. The arrays are strategically placed around the periphery of the lens, leaving the central optical zone unobstructed to preserve normal visual functionality. This peripheral positioning enables the lens to maintain its primary role in vision correction while adding advanced features such as drug delivery or biosensing. For instance, drug-loaded arrays can gradually release therapeutic agents to the ocular surface, providing a dual-purpose lens that combines vision correction with localized treatment. In some ophthalmic applications, the ophthalmic drug is loaded via solvent diffusion just prior to use by the user or physician. Various embodiments for drug loading in the array via aqueous/solvent diffusion has been disclosed in earlier section and such methods can be used to load/infuse the drug just before using the ophthalmic device.

The following non-limiting examples are intended to illustrate the inventive concepts disclosed in this document. Those skilled in the art will appreciate that modifications can be made to these examples, drawings, illustrations, specifications and claims, which are intended to fall within the scope of the present invention.

### Materials and Methods

### Materials

All chemicals used were of high purity grade. Gelatin powder, methacrylic anhydride, triethylamine, 2-hydroxy-4-(hydroxyethoxy)-2-methylpropiophenone, vinyl pyrrolidone, PEG35K, toluene, triethylamine, n-hexane, polyethylene glycol, hexamethylene diisocyanate, 2-hydroxyethyl acrylate, dimethyl sulfoxide (DMSO), magnesium carbonate, dibutyltin dilaurate, polyvinyl alcohol, coumarin dye, psyllium husk, acryloxy terminated ethylene oxide-dimethylsiloxane-ethylene oxide aba block copolymer (DBE-U-12), stannous octoate, dl-lactide, acrylic acid, sodium hydroxide, disodium hydrogen phosphate, sodium dihydrogen phosphate, sodium chloride and sucrose acetate isobutyrate were sourced from Loba Chemie, India; Tokyo Chemicals Limited; Molychem, India; Lancaster synthesis; Sigma Aldrich; Tokyo Chemicals Limited; Gelest, USA; Alfa Aesar USA; Merck India; SD Fine and Chemicals Limited India. Eosin Y, ethyl eosin, acrylic acid n-hydroxysuccinimide ester, polyethylene glycol, polyethylene oxide and polypropylene oxide block copolymers, 2-Hydroxy-1-[4-(2-hydroxyethoxy)phenyl]-2-methyl-1-propanone (Irgacure 2959), n-hydroxysuccinimide, were purchased from Sigma-Aldrich. Multifunctional hydroxyl and amine-terminated polyethylene glycols were purchased from Dow Chemicals, BASF, Huntsman, Texaco, Creative PEG Works. Disuccinimidyl glutarate (DSG), sulfosuccinimidyl suberate (DSS) and N-hydroxysulfosuccinimide (NHS) were purchased from Pierce or Sigma-Aldrich. PEG based monofunctional, difunctional, trifunctional, tetra-functional and octa-functional NHS esters and other crosslinkers/derivatives were sourced from commercial sources like Creative PEG Works, Winston Salem, NC, USA; Jenkem Technology USA, Allen, TX, USA.; BOC Sciences, Shirley, NY USA; Laysan Bio, Inc. Arab, AL; NOF America, Corporation, White Plains NY USA and Sigma Aldrich, USA. They can also be synthesized by procedures described in illustrative embodiments reported in this invention or using methods known in the art. Trilysine acetate salt (Cat No 402495) was purchased from Bachem Americas. Lipase (Type VII, Cat No L1754) is obtained from Sigma. Various monomers are obtained from commercial sources like Sigma Aldrich, Polysciences, Sartomer USA, Exton PA, and Gelest Morrisville, PA, and Scientific Polymer Products, Ontario NY.

Many crosslinked products are generally added/filled with inorganic fillers like silica (HI-SIL® silica product from PPG) to improve mechanical and other physical properties. In addition to fillers, crosslinked compositions can comprise other additives like antioxidants, stabilizers, coloring pigments and the like. Many reagents, solvents can be purchased from commercial sources like, by way of example, and not limitation, Polysciences, Fluka, ICN, Sigma-Aldrich and the like. SYLGARD™ 184 Silicone Elastomer Kit is purchased from local suppliers to cast silicone rubber molds. Small laboratory equipment and medical supplies can be purchased from Fisher or Cole-Parmer.

Cell culture experiments were performed using a standard mammalian tissue culture laboratory or microbiology laboratory capable of handling and growing mammalian and human cell cultures. Unless mentioned, live cells, blood or plasma or serum related experiments are done in a sterile manner. Commercial sources like Sigma-Aldrich, Gibco, VWR, Thermo Fisher and the like are used to obtain cell culture medium. American Type Culture Collection (ATCC) and other commercial vendors are used to obtain mammalian cells. A clean room or sterile hoods are generally used to conduct the live cell culture experiments and to maintain sterility.

Long UV light lamps (model UV-300, Wenzhou Aurora Technology Company Ltd., Wenzhou, China, 365 nm light with intensity around 26,500 μW/cm² when held 1 inch above the surface) or Black-Ray UV lamp, (360 nm light filter, about 10000 mW/cm2 intensity when held about 1 inch above the surface) is used. Argon laser emitting at 513 nm is used for visible light polymerization initiated by Eosin. Solid state laser sources emitting at 520 nm, 532 nm, 445 nm, 488 nm with total power output from 0.05 Watts to 25 Watts can be purchased from Laser Lab Source, Bozeman, MT and other commercial sources.

The size, shape and distribution of microparticles or microspheres can be assessed by laboratory microscopes with varying magnification power or with scanning electron microscopes. The distribution of various sizes in a mixture is assessed by a particle size analyzer.

Molecular weight is determined by gel permeation chromatography (GPC); or NMR (proton) or mass spectrophotometry.

Biocompatibility is assessed by several USP tests recommended by the US FDA. People skilled in the art know that commercial laboratories are available which perform routine biocompatibility tests for their clients. These tests include implantation and subsequent histological testing for immunogenicity, inflammation, drug release and degradation studies.

In vitro degradation of the polymers/gels is monitored gravimetrically at 37 degree C., in aqueous buffered medium like, by way of example, and not limitation, phosphate buffered saline (pH 7.4). In vivo biocompatibility and degradation life times are assessed after subcutaneous implantation. The implant is surgically inserted into the animal body. The degradation of the implant over time is monitored gravimetrically or by chemical analysis. The biocompatibility of the implant is assessed by standard histological techniques. Chemical analysis like, by way of example, and not limitation, structure determination is done using nuclear magnetic resonance (proton and carbon-13), Raman spectroscopy, x-ray diffraction and infrared spectroscopy.

Chemical analysis such as, by way of example, and not limitation, structure determination is done using nuclear magnetic resonance (proton and carbon-13), Raman spectroscopy, x-ray diffraction and infrared spectroscopy.

High-pressure liquid chromatography or UV-visible spectrophotometry is used to determine drug elution profiles.

Thermal characterization like, by way of example, and not limitation, melting point, shrink temperature and glass transition temperature are done by differential scanning calorimetric analysis. The aqueous solution properties like, by way of example, and not limitation, self-assembly, micelle formation and gel formation are determined by fluorescence spectroscopy, UV-visible spectroscopy and laser light scattering instruments.

Drug release studies are conducted in PBS under sink conditions at 37 degrees C. and the drug elution is monitored by HPLC or UV-VIS spectrophotometer.

### Example 1

Syntheses of Various Precursors/Macromonomers and their Solutions.
Preparation of Phosphate Buffered Solution (PBS, pH-7.4)

1.195 g disodium hydrogen phosphate, 0.091 g sodium dihydrogen phosphate, and 4.052 g sodium chloride were dissolved into distilled water and made to 500 ml. The pH of the solution was then adjusted to around 7.4 by using dilute hydrochloric acid or sodium hydroxide while monitoring using a pH meter.

### Preparation of About 1% Polyvinyl Alcohol (PVA) Solution.

5.012 g PVA was dissolved in 500 ml of distilled water. The solution was used as a mobile phase in the droplet generator for the preparation of crosslinked microspheres.

### Preparation of Photoinitiator Solution (PS).

0.501 g of 2-Hydroxy-4-(hydroxyethoxy)-2-methylpropiophenone (Irgacure 2959) was dissolved in 1 ml vinyl pyrrolidone. Small quantity of this solution was mixed with monomer/macromonomer solution and then irradiated at 360 nm to initiate polymerization.

### Example 1A

Synthesis of Precursors that Form Crosslinked Materials.

Precursors that form crosslinked material by chain growth or free radical polymerization.

### Synthesis of Gelatin Methacrylate (GM).

0.199 g of gelatin was dissolved in 1 ml of buffer (pH 9), 20 microliters of methacrylic anhydride was added followed by 20 microliters of triethylamine. Reaction mixture was kept at 40 degree C. in the oven for 1 hour 20 mins. In some embodiments this solution was used directly which has unreacted methacrylic acid and/or methacrylic anhydride monomer as impurities. In most cases, the solution was subjected to dialysis (10000 g/mol cutoff membrane) for 72 hours with daily changes of water medium to remove small molecular weight components from the reaction mixture (with molecular weight less than 10000 g/mole) in distilled water followed by separation of gelatin methacrylate by lyophilization or air drying followed by vacuum drying while protecting from ambient light.

1 ml of 10 percent of gelatin methacrylate in the PBS solution was added with a small amount of red ink to provide a red color. 10 microliters of photoinitiator solution (PS) were added to the red gelatin solution (referred as red colored gelatin methacrylate photopolymerizable precursor, RGM). About 20 microliter solution of RGM was exposed to long UV light (365 nm) for 1 minute and the solution formed a red colored crosslinked gelatin methacrylate hydrogel. Using a similar procedure as above, red color ink from the RGM solution was substituted with blue, green and purple colored ink to obtain blue colored precursor solution (BGM), green colored precursor solution (GGM), purple colored precursor solution (PGM) and no color precursor solution (NGM) respectively and all solutions polymerized and crosslinked within 120 seconds upon exposure to long UV light.

### Example 1B

### Synthesis of PEG-35K-Methacrylate (PEG35KDMA).

10.004 g PEG with 35000 g/mole molecular weight (PEG35K) was dissolved in 100 ml of toluene in a round bottom flask, 20 ml of toluene was distilled out from reaction mixture under an inert (nitrogen) atmosphere. The solution was then cooled to room temperature and 0.133 ml triethylamine followed by 0.170 ml methacrylic anhydride was added. This mixture was then refluxed for 2 hours under inert conditions. After 2 hours, the reaction mixture was cooled to room temperature and then poured into 150 ml cold hexane while stirring continuously. The precipitated monomer (PEG35KDMA) was removed by filtration followed by vacuum drying under vacuum for 16 hours.

Synthesis of Polyethylene Glycol 20000 g/Mole (PEG20K) Urethane Acrylate (PEG20KUA).

3.001 g PEG20K was dissolved in 130 ml dry toluene in a round bottom flask. Toluene was distilled out under an inert (nitrogen) atmosphere and the initial 30 ml portion was discarded. 50 ml dry toluene was collected in a 250 ml beaker and 0.145 ml of hexamethylene diisocyanate was added to the dry toluene, followed by a drop of dibutyltin dilaurate. The PEG-20K solution (cooled to RT) was added slowly and with constant stirring to this mixture and then refluxed under nitrogen for 2.5 hours. The reaction mixture was cooled to RT and 105 microliter of 2-Hydroxyethyl acrylate was added to it. The reaction mixture was again refluxed under nitrogen for 2.5 hours. Reaction mixture was cooled to RT and decanted in a large excess of cold hexane. Precipitated product (PEG20KUA) was filtered and dried in vacuum overnight. Using a similar procedure PEG-35K urethane acrylate (PEG35KUA) was prepared using 5.016 g PEG35K, 0.140 ml of hexamethylene diisocyanate, 100 microliter of 2-Hydroxyethyl acrylate, drop of dibutyltin dilaurate and toluene as a solvent.

Using a similar procedure as used for PEG20KUA and PEG35KUA, other PEG based urethane macromonomers were synthesized. PEG with molecular weights 1000, 2000, 3000, 4000, 10000 and 100000 g/mole were used to obtain PEG1KUA, PEG2KUA, PEG3KUA, PEG4KUA, PEG10KUA, PEG100KUA macromonomers respectively. Pluronic F127, Pluronic F68 were also used in place of PEG to obtain urethane acrylate derivatives F127UA and F68UA respectively.

### Example 1C

### Synthesis of PEG-Polylactone Biodegradable Macromonomer (PEG10KL5A)

Part 1: In 250 ml flask 30 g PEG 10000 was dissolved in 100 ml toluene. 20-30 ml toluene was distilled out under nitrogen atmosphere. The solution was cooled to room temperature and 4.320 g dl-lactide (3,6-Dimethyl-1,4-dioxane-2,5-dione) and 0.1 ml stannous octoate were added sequentially in the solution under nitrogen at room temperature. The solution mixture was refluxed for 4 hours under nitrogen atmosphere, cooled and then poured in cold hexane to separate synthesized product. Hexane was removed by decantation. The synthesized product PEG1000-lactide copolymer (PEG10KL5) was dried under vacuum overnight and used in the next reaction.

Part 2: End-capping of PEG10KL5 with polymerizable or crosslinkable group.

30 g of polyethylene glycol lactate polymer (PEG10KL5) prepared above was dissolved in 250 ml dry toluene. About 50 ml of toluene was distilled out under an argon atmosphere to remove traces of water from the reaction mixture. The solution was cooled to room temperature, 0.59 g of triethylamine and 0.52 g acryloyl chloride were added. The reaction mixture was then warmed to 50-60 degree C. and stirred for 30 minutes at 50-60 degree C.; cooled to room temperature and filtered. The product (macromonomer) was precipitated by adding the filtrate to 2,000 ml cold ether. The precipitated macromonomer (PEG10K-LACTATE-5-diacrylate; PEG10KL5A) was recovered by filtration. It was then dried under a vacuum for 12 hours at 50 degree C. The macromonomer synthesized has PEG 10000 Daltons as a central block that is extended with polylactide as a biodegradable block (about five lactide units on both sides of PEG chain) and then terminated with an acrylate polymerizable group. The polylactate group is between acrylate and PEG.

In another modification as above, 30 g of PEG 20000 was reacted with 2.160 g dl-lactide using 30 mg of stannous octoate in the first part to produce PEG 20000-lactate copolymer (PEG20K5L). 30 g of PEG20KL5 was then reacted with and 0.50 g acryloyl chloride and 0.59 g triethylamine in the second part to produce PEG20K-lactate-diacrylate (PEG20KL5A).

In another modification as above, 30 g of PEG 35000 was reacted with 1.234 g dl-lactide using 30 mg of stannous octoate in the first part to produce PEG 35000-lactate copolymer (PEG35KL5). 30 g of PEG35KL5 was reacted with 0.3 g acryloyl chloride and 0.3 g triethylamine in the second part to produce PEG35K-lactate-diacrylate (PEG35KL5A).

In another modification as above, 30 g Pluronic F127 was reacted with 3.6 g dl-lactide in the first part to produce Pluronic F127-lactate copolymer (F127L5) which was then reacted with 0.48 g acryloyl chloride and 0.54 g triethylamine in the second part to produce Pluronic F127-lactate-diacrylate (F127L5A).

For additional examples of macromonomers, biodegradable crosslinked compositions and free radical polymerization initiating systems please refer to U.S. Pat. Nos. 5,410,016, 5,573,934, 6,201,065, 6,566,406, 9,023,379, 6,387,977, 9,789,073, and cited art therein; cited herein for reference only.

### Example 1D1

### PEG-Polylactone Copolymer as a Biodegradable Carrier for Drug/Visualization Agent.

Synthesis of PEG 10000-Lactide Copolymer with 1:2 Ratio of PEG to Lactone Weight Ratio (PEG10KL-1-2)

In 250 ml flask 1.0214 g PEG 10000 was dissolved in 100 ml toluene. 20-30 ml toluene was distilled out under nitrogen atmosphere. The solution was cooled to room temperature and 2.011 g Dl-lactide (3,6-Dimethyl-1,4-dioxane-2,5-dione) and 0.1 ml stannous octoate were added sequentially in the solution under nitrogen at room temperature. The solution mixture was refluxed for 4 hours under nitrogen atmosphere. Then cooled and poured in cold hexane to separate synthesized products. Hexane was removed by decantation. The synthesized product was dried under vacuum for 16 hours and kept in a desiccator.

### Example 1D2

### Synthesis of PEG 2000-Lactide Copolymer Thermosensitive Biodegradable Carrier (PEG2KL-1-2)

Synthesis of PEG 2000-Lactide Copolymer with 1:2 Ratio of PEG to Lactone

A 250 ml flask equipped with heating mantle and condenser was added 2.551 g PEG 2000 followed by 100 ml toluene and stirred until complete dissolution. About 30 ml toluene was distilled out under nitrogen atmosphere. The solution was cooled to room temperature and 5.0 g dl-lactide and 0.2 ml stannous octoate were added in solution and the mixture was refluxed for 4 h under nitrogen atmosphere. The solution was cooled and poured in cold hexane to precipitate the PEG-polylactone copolymer (PEG2KL-1-2) product. Hexane was removed by decantation and additional 50 ml cold hexane was used for washing the product. The product was filtered and dried in a vacuum oven overnight. In a 10 ml glass vial, 0.212 g of dry product was dissolved in ice-cold 1.0 ml of PBS. The solution was tested for thermoreversible gel formation. Briefly, the solution vial was kept in an oven maintained at 40-degree C. The solution formed a gel at 40 degree C. (the contents of the vial did not flow when the vial was inverted). The vial is taken out and cooled to zero-degree C. The gel formed is converted back into a polymer solution (solution flows when the vial is inverted) indicating thermoreversible gelation nature of the polymer.

### Example 1E

Precursors that Form Crosslinked Polymers by Condensation Polymerization.

### Polymerization of Precursors Comprising Electrophilic and Nucleophilic Groups to Produce Crosslinked Gels.

In a 15 ml glass vial, 1 g of 4 arm polyethylene glycol with molecular weight 10000 g/mole and terminal amino groups per arm (PEG10K4 ARM tetramine) was dissolved in 9 ml of PBS (pH 7.4). In another 15 ml glass vial, 1 g of 4 arm polyethylene glycol with molecular weight 10000 g/mole glutarate ester terminated with n-hydroxysuccinimide (PEG10K4ARM glutarate NHS ester) prepared as above was dissolved in 9 ml of PBS (pH 7.4). 0.5 ml of PEG10K4 ARM tetramine and 0.5 ml of PEG10K4ARM glutarate NHS ester solutions were mixed in 2 ml glass vial and immediately loaded into several 3 mm diameter and 1 mm height cylindrical cavity molds. The pH (pH range 6 to 8, substantially equimolar) of the reaction medium was adjusted to slow the crosslinking reaction so that there would be sufficient time to pour the mixture in the mold before crosslinking. The formed gel was soft and elastic, indicating effective crosslinking.

In a 15 ml glass vial, 1 g of thermosensitive biodegradable polymer (PEG2KL-1-2) was dissolved in 3 ml of cold PBS solution. To this solution 0.3 g of PEG10K4ARM tetramine was added and shaken until complete dissolution. In a 15 ml glass vial, 0.3 g of commercially purchased glutarate NHS ester (PEG10K4ARM) was dissolved in 3 ml of PBS. 100 microliters of each of the above solutions were mixed and added in 3 mm mold cavities to form a crosslinked polymer at 0 to 10 degree C. with addition of one drop of triethanolamine solution (10 percent in PBS) as a catalyst. The crosslinked gel had thermosensitive biodegradable polymer (PEG2KL-1-2) as a drug carrier entrapped in the crosslinked polymer. 102.1 mg of glycerol ethoxylate (Molecular weight 1000), 0.2 ml of Toluene, 19 μl of glutaryl chloride and 42.0 μl of triethylamine were mixed well and filled in mold cavities. After 30 minutes, sticky organic solvent gels were observed in the mold cavities. In another example, 37.5 mg of glycerol ethoxylate, 0.1 ml of toluene, 9 μl of glutaryl chloride were mixed and this mixture was poured into the mold cavity and then 33 μl of triethylamine was added and mixed.

Delayed addition of triethylamine helped to delay the gelation which helped to give sufficient workup time to fill the cavities in the mold before crosslinking and gelation. The gel was removed from the mold and washed with water to remove triethylamine hydrochloride and other unreacted products. In another embodiment as above, 102.1 mg of glycerol ethoxylate and 50 mg of PLGA (molecular weight, acetate endcapped) were reacted with glutaryl chloride as above to prepare an organic solvent gel (organogel) with PLGA entrapped in the gel. The stoichiometry between electrophilic and nucleophilic groups varied until gelation time was obtained between 2 to 10 minutes. Equivalent or very close to equivalent stoichiometry between electrophilic and nucleophilic groups in the precursors was most preferred for effective polymerization and crosslinking of precursors.

For additional examples of crosslinked polymers made via condensation polymerization, please refer to U.S. Pat. Nos. 9,498,557, 7,009,034, 6,201,065 and references cited therein, cited herein for reference only.

### Example 2

### Preparation of Composite Microspheres.

### Example 2A

Preparation of Microspheres Comprising Crosslinked Hydrogel and Hydrophobic or Hydrophilic Biodegradable Polymer as a Drug Carrier Wherein Drug is Incorporated in Crosslinked Polymer and/or Biodegradable Polymer Carrier.

Organic solvent gelation method for preparing (organogel preparation) biodegradable microspheres with drug/visualization agents.

In a 20 ml glass vial, 0.250 g of PEG10KUA, 1.505 g PLGA were dissolved in 5 ml dichloromethane (DCM). 20 microliters of photoinitiator solution were added. About 20 microliters of the solution was exposed to long UV light and formed a gel within 60 seconds.

This solution was filled in a 10 ml syringe and the syringe was attached to the syringe pump. 1 percent polyvinyl alcohol was filled in a 60 ml syringe and the syringe was attached to a second syringe pump. Microspheres were prepared using 1% PVA solution as mobile phase and PEG20KUA-PLGA solution using an inhouse created microfluidic droplet generator. The syringes were connected to a microfluidic chip. The microfluidic chip was primed with PVA solution to remove air bubbles. The PVA solution was pumped at 100 ml per hour and the precursor PLGA solution was pumped at 2 ml per hour through the chip. The solution droplets were collected in a glass beaker which was exposed to long UV light. In some embodiments, droplets were irradiated in line as they formed and/or also collecting them in the beaker. The collected microsphere underwent polymerization and crosslinking and maintained their spherical shape and size without agglomeration or breakage of solution droplets. Microspheres (average size 446 microns) were then washed with water and dried to remove dichloromethane. A small quantity of microspheres was taken in a 10 ml beaker and washed with 1 ml DCM. After decantation, the microspheres were soaked in 1 ml DCM containing a few micrograms of coumarin as a model drug for about 10 minutes. The excess solvent was then removed, microspheres were washed with two portions of 1 ml each of methanol and then once with distilled water. The microspheres were stored in water for 24 h and photographed. The crosslinked PEG10KUA swells in water and was seen as a clear transparent hydrogel surrounding the core of PLGA with fluorescent properties (fluorescence due to encapsulated coumarin in the PLGA). Coumarin is also used herein as a model hydrophobic drug.

### Example 2B

Preparation of Microspheres Comprising Crosslinked Hydrogel with Liquid Polymeric or Non-Polymeric Liquid Carrier.

### Preparation of Microspheres Comprising Photo-Crosslinked PEG Based Hydrogel and Sucrose Acetate Isobutyrate as a Liquid Carrier.

Using a similar method as above, sucrose acetate isobutyrate was used as a liquid carrier in place of PLGA polymeric carrier. A solution of 10% PEG20KUA along with photoinitiator and 5 percent sucrose acetate isobutyrate in DCM was used as carrier and 1% PVA solution as a mobile phase. A 22 G IV cannula with built in 3-way stop cock was used instead of microfluidic chip. The needle-in-needle arrangement in the IV cannula wherein the inner tube is fed with macromonomer solution in DCM (flow rate 1.5 ml per hour) and outer tube is fed with PVA solution (flow rate 100 ml per hour). The droplets of DCM solution were collected and irradiated with UV light. Changing flow rates of organic macromonomer solution and mobile phase solution droplets of various sizes were obtained. The outlet and inner diameters of the device were also varied to obtain desired size microspheres. Solvent was removed from the crosslinked microspheres with trapped sucrose acetate as a liquid carrier was obtained. Drugs can be added in DCM solution before polymerization. Drugs also can be added later via solvent diffusion technique. The microspheres can be suspended in saline and injected in the body for sustained drug delivery.

### Example 2C

### Preparation of Microspheres Comprising Crosslinked Hydrogel and Thermosensitive Polymer.

Preparation of microspheres comprising photo-crosslinked PEG based hydrogel and thermosensitive biodegradable gel wherein the thermosensitive polymer exists as a gel in the crosslinked polymer.

Using a similar procedure as in 8 A and/or B, microspheres with PEG2KL-1-2 as a carrier were made in place of PLGA polymer. The polymer PEG2KL-1-2 has thermosensitive gelation properties wherein it forms a thermosensitive gel around 37 degree C. at a concentration of 10-40 percent in water or in aqueous buffer like PBS. The experimental conditions used in above experiments wherein the PEG10KL-1-2 polymer was present as a thermosensitive gel at 37 degree C. in PBS inside the microspheres, simulating the in vivo environment found inside human or animal body.

### Example 2D

### Microspheres by Solid State or Substantially Solid State Polymerization of Precursors.

### Microspheres by Spray Drying of Precursor Solutions.

### Microspheres by Spray Drying of Precursor Solutions in Volatile Organic Solvents.

In a 20 ml glass vial, 0.252 g of PEG10KUA, 1.517 g PLGA were dissolved in 5 ml dichloromethane (DCM). 20 microliters of photoinitiator solution was added. About 20 microliters of the solution was exposed to long UV light and formed a gel within 60 seconds.

The solution was sprayed using the standard laboratory sprayer and the substantially dry microparticles/microspheres (about 30-70 microns in size with small/trace amount of solvent) were collected in a beaker. The collected microspheres were then exposed to long UV light for 10 minutes to polymerize and crosslink particles. The crosslinked particles were then further dried to remove the solvent. The crosslinked PEG based particles were stored in the refrigerator and in dry atmospheres until further use. The particles thus formed had crosslinked PEG based material with PLGA entrapped in the particles. The particles are then incubated in drug solution for desired amount of time until sufficient drug is incorporated in the crosslinked particles without substantial loss of PLGA from the microparticles via diffusion. The solvent is removed by lyophilization or vacuum drying and the drug loaded microspheres are stored until further use.

In another modification of the above example, 0.242 g of PEG10KUA, 1.498 g PLGA and 148 mg of bupivacaine are dissolved in 5 ml dichloromethane (DCM). 20 microliters of photoinitiator solution is added and the solution is sprayed and crosslinked as described above.

These crosslinked microspheres have drug bupivacaine encapsulated in the crosslinked PEG based polymer as well as in PLGA entrapped in the crosslinked macromonomer (PEG10KUA).

### Example 3

### Preparation of Biodegradable Beads/Disks and Buttons of Various Sizes and Holes.

### Example 3A

Rods with a diameter of 2 mm, composed of PLGA (50:50) or polylactide (PLA), are commercially obtained or extruded from raw polymer materials. These rods are cut longitudinally to produce disks with a diameter and height of 2 mm each. Alternatively, biodegradable polymers such as polylactide are injection molded or 3D printed using commercially available equipment. Low-melting biodegradable polymers (melting point below 100° C.), such as PEG-polylactone copolymers, are melted within molds of the desired size and shape, or their powders are sintered near the melting point to form beads and disks of various sizes, shapes, and holes. In another modification, biodegradable polymer tubes, such as PLGA tubes with an outer diameter of 2 mm and an inner diameter of 1 mm, are procured or custom-extruded and cut into 2 mm sections. These cylindrical segments, featuring a 1 mm central hole, are utilized as-is in making arrays or coated with drugs or visualization agents for subsequent array formation.

### Example 3B

Beads with Holes

Using a mechanical microdrill equipped with a 300-micron drill bit (typically used for printed circuit boards), a 300-micron hole is drilled into a disk of 2 mm diameter and 2 mm height prepared as described above. Alternatively, two holes of 300 microns each are drilled around the center, with a 500-micron distance maintained between them.

### Example 3C-1

### Crosslinked Polymer Coating Via Free Radical Polymerization

Beads are coated with a biodegradable polymer, with or without a drug or visualization agent. In a 20 ml glass vial, 0.494 g of PEG20KUA, 0.251 g of PEG10KL-1-2, and 10 mg of bupivacaine base are dissolved in 5 ml of methanol (a non-solvent for the bead polymer). Fifty microliters of a photoinitiator solution are added. A disk with a 2 mm diameter and 1 mm thickness is dip-coated in the PEG20KUA solution and exposed to UV light for 2 minutes to polymerize the precursor on the bead surface. Since methanol does not dissolve the bead material, it does not affect the surface during crosslinking. The coating comprises a crosslinked polymer, PEG2K, as a biodegradable carrier, and bupivacaine base as the drug. A 100-micron hole is drilled at the center of the coated disk using an infrared laser.

In an alternative embodiment, disks with one or more holes are dip-coated or spray-coated, ensuring the holes remain substantially unobstructed after coating.

### Example 3C-2

### Crosslinked Polymer Produced by Condensation Polymerization.

In a 15 ml glass vial, 1 g of 4-arm polyethylene glycol (PEG10K4 ARM tetramine) with terminal amino groups (molecular weight: 10,000 g/mol) and 100 mg of bupivacaine hydrochloride are dissolved in 9 ml of PBS (pH 7.4). Separately, 1 g of 4-arm polyethylene glycol glutarate ester terminated with N-hydroxysuccinimide (PEG10K4ARM glutarate NHS ester) is dissolved in 9 ml of PBS (pH 7.4). Equal volumes (1 ml each) of these solutions are loaded into a double-barrel syringe fitted with a sprayer instead of a needle. The mixed solution is sprayed onto the surface of PLGA beads prepared as described previously, resulting in crosslinking on the bead surface and drug entrapment. Coating thickness ranges from 5 microns to 2 mm, preferably between 10 microns and 1 mm.

### Example 3C-3

Coated Array Beads/Elements with a Drug or Visualization Agent.
Coating with Synthetic Non-Crosslinked Drug Carrier.

### Coating of PEG-Polylactone Copolymer on Bead Surface

In a 20 ml glass vial, 1 g of PEG-polylactone copolymer (PEG2KL-1-2) is dissolved in 10 ml of methanol, followed by the addition of 100 mg of rifampin. PLGA beads, prepared as per Example 3A, are added and incubated with constant stirring for 30 minutes. The beads are then removed, and the solvent is evaporated by air drying, followed by vacuum drying. The process leaves a PEG-polylactone copolymer coating containing the drug on the bead surface.

Alternatively, cold aqueous solutions of PEG2KL-1-2 can also be used with drug and water is removed via lyophilization.

### Example 3C-4

Coated Beads/Elements with a Drug or Visualization Agent.
Coating with Natural Non-Crosslinked Drug Carrier.

### Coating of Enzymatically Degradable Polymer (Gelatin)

In a 20 ml glass vial, 10 g of gelatin is dissolved in 100 ml of hot water. After cooling to room temperature (25-30° C.), 1 g of chlorhexidine acetate is added. PLGA beads, prepared as described in Example 3A, are added and incubated with constant stirring for 30 minutes. The beads are then removed and dried either by air or lyophilization, resulting in gelatin-coated beads with chlorhexidine acetate on their surfaces.

### Example 3C-5

### Coating on Array Devices

Ten PLGA beads (50:50, molecular weight approximately 70,000 g/mol) with a 2 mm diameter and 1 mm height, each featuring a central hole (0.3 mm diameter), are procured or prepared as previously described. A cut gut suture is threaded through the central holes of the beads, secured with knots on both sides to prevent migration, leaving 5 cm of suture thread on each end. The assembled array is incubated in a methanol solution of PEG2KL-1-2 for 30 minutes, then air-dried to allow the coating to form on both the beads and the suture thread.

### Example 3C-6

### Solvent Softened Beads for Threading

Ten PLGA beads (50:50, molecular weight approximately 70,000 g/mol) with a 2 mm diameter and 1 mm height, without holes, are procured. These beads are incubated in a 50:50 mixture of methanol (a non-solvent for PLGA) and tetrahydrofuran (THF, a solvent for PLGA) at ambient temperature. The incubation conditions (time, solvent mixture composition, and temperature) are adjusted to achieve the desired bead softness. After achieving a desired softness, A cut gut suture is threaded through the softened beads, secured with knots on both sides to prevent migration, leaving 5 cm of suture thread on each end. After air and vacuum drying, the array device is coated with drugs or visualization agents as previously described.

### Example 3C-7

### Biodegradable Metal Based Beads and Arrays.

A 200 mm length, 500 mm width and 0.25 mm thick magnesium alloy (AZ 31) foil containing About 96 percent magnesium, 3 percent Aluminum and 1 percent zinc is procured from Goodfellow, Pittsburgh PA (Goodfellow Product UOM Code: 963-383-57). From the foil, about thirty 3 mm diameter and 0.25 mm height disks and are cut. A center hole of 0.5 mm dia hole drilled at the center for each disk. In 1 20 ml glass vial, 1 g of PLGA, molecular weight 70000 g/mole, 100 mg bupivacaine are dissolved in 9 g of THF. About 10 AZ31 disks are incubated in PLGA solution and taken out after 10 minutes of incubation and air dried, followed by vacuum drying. 10 PLGA coated disks with bupivacaine base are threaded into Vicryl, polyglactin 910 suture thread. The thread is looped into disk hole and disk is locked into the thread with two knots. Ten disks are threaded and attached to the suture. A thread length of 10 mm is kept between each disk and for terminal disk, a length of 20 cm on both sides is kept. The array device thus prepared is sterilized using ethylene oxide and stored until use. For additional examples of magnesium based materials and device, please refer to U.S. patent Ser. No. 10/624,865 and references therein, cited herein for reference only.

### Example 3D

### Drug Delivery Devices Made Using Coated or Crosslinked Beads/Disks.

### Suture Threaded Crosslinked Drug Delivery Device.

Precursor Solution with Bupivacaine as a Model Drug.

0.052 g of PEG20KUA, 0.0515 g PLGA (50:50, molecular weight 20000 g/mole) and 0.015 g of bupivacaine base were dissolved in 0.5 ml dimethyl sulfoxide (DMSO). After complete dissolution, 10 microliter photoinitiator was added and one drop of the solution polymerized within 60 seconds.

Precursor solution with Iodixanol as a model visualization agent (x-ray contrast agent). 0.0513 g of PEG20KUA, 0.0510 g PLGA (50:50, molecular weight 20000 g/mole) and 0.0151 g of Iodixanol were dissolved in 0.5 ml DMSO. 10 microliter photoinitiator solution was added and one drop the solution polymerized within 60 seconds. Precursor with biodegradable carrier with no drug or visualization agent (control solution) 0.051 g of PEG20KUA and 0.052 g PLGA (50:50, molecular weight 20000 g/mole) were dissolved in 0.5 ml DMSO. 10 microliter photoinitiator solution was added and one drop the solution polymerized within 60 seconds. In a polypropylene mold with 3 mm diameter and 1 mm depth, solutions prepared above were filled completely and exposed to long UV light for 2 minutes to polymerize and crosslink to form 3 mm diameter and 1 mm height disk/cylinders. 4 gels from each of the compositions were prepared. The disks with no drug or visualization were incubated in a DMSO solution of rifampin (5 percent weight/volume) for 5 minutes to infuse rifampin in the disk. Disks with bupivacaine were stained with methylene blue solution to impart blue color to the disks.

Iodixanol comprising gels were used as is (uncolored or semitransparent).

### Example 3D1

### Threading Cylindrical Disks Through the Side.

Devices with Flexible Thread Between Disks/Beads.

### Use of Organogel Disks for Making the Device.

Catgut suture (Ethicon, India, size 4.0) with a needle was used to thread the all 12 discs (crosslinked organogel with solvent DMSO). 4 red rifampin comprising disks, 4 blue colored bupivacaine disks and 4 Iodixanol comprising disks were used (as described in previous Example 3D) to form a threaded multi-compositional organogel based controlled drug delivery device. All disks were organogel in nature (crosslinked polymer swollen in biocompatible water soluble organic solvent like DMSO) which makes the disks soft and easy to thread. Suture needle along with thread was inserted in the red disk via sideways (perpendicular to disk/bead axis) until it comes out on the other side of the disk along with the thread. A knot is placed before and after the disk so that disk does not move after array has been made. Second uncolored disk was inserted similar to the first disk and a 2-5 mm thread spacing was kept between the two disks. A third blue color bead was inserted with 2-5 mm thread distance between them. This sequence was repeated three times to make a drug delivery device with twelve disks. A suture length of about 30 mm on both ends was used. The drug delivery device thus created has bupivacaine as illustrative local anesthetic agent, rifampin as illustrative antibiotic and Iodixanol as illustrative visualization agent. The solvent from the disks can be removed under vacuum if desired. During closure of surgical wound, the drug delivery device can be implanted in the surgical incision/wound prior to closure and then sutured in place to immobilize the device at the wound site. The disk used can be swollen in biocompatible solvent such as DMSO or without solvent.

The disks in the device release antibiotics locally to control infection, release local anesthetic to reduce pain associated with surgical wounds. The x-ray contact agent can help to visualize the device using x-ray imaging if needed. Solvent DMSO in disks if used is safely removed in the tissue upon implantation.

In another modification of the above example, the solvent DMSO from organogel disks was first completely removed by air drying followed by vacuum drying and then disks were threaded in a desired sequence.

In another modification of the above example device, disks with rifampin only were used to make a device. Similarly, devices with disks comprising bupivacaine and Iodixanol were made separately. During implantation, 3 devices each comprising bupivacaine, rifampin and Iodixanol respectively were used.

In another modification, disks made via condensation polymerization reaction of precursors (precursor with nucleophilic and electrophilic groups) were used to make the threaded device as described earlier.

### Example 3D2

### Threading Cylindrical Disks Through the Side.

Devices with Little or No Space Between Disks/Beads

12 beads with three different compositions as described in above (3D1) were threaded as described above without any or very little space between the disks (closely packed beads). To prevent movement of beads, a knot was placed before the first disk and at the end of the last disk (12th disk). The knot prevents movement of beads along the thread. Terminal ends have about 30 mm of suture length which could be used for suturing the device to the tissue.

### Example 3D3-1

### Threading Cylindrical Disks Through the Side.

Devices with space between disks/beads and several knots to prevent disk migration. 12 beads with three different compositions as described in above (3D1) were made. For all 12 discs, a knot was placed at the entry point of the needle on the disk and also after the needle exit from the disk (knot on both ends). The knot before and after the disk is used to prevent movement/migration of disks after the device has been prepared. Terminal ends had about 30 mm of suture only that could be used for suturing the device to the tissue.

### Example 3D3-2

### Threading Cylindrical Disks Wherein Suture Thread is Parallel to the Cylindrical Axis.

Catgut suture (Ethicon, India size 4.0) was used to thread the all 12 discs (crosslinked organogel with solvent DMSO). 4 red rifampin comprising disks, 4 blue colored bupivacaine disks and 4 Iodixanol comprising disks were used to form a threaded multi-compositional organogel based controlled drug delivery device. All disks were organogel in nature (crosslinked polymer swollen in biocompatible water soluble organic solvent like DMSO) which made the disks soft and easy to thread. Suture needle as was inserted in the red disk from bottom to top side via circular planer surface (center of the plane, aligned to disk/bead axis). Second uncolored disk was inserted similar to the first disk and a 2-5 mm thread spacing was kept between the two disks. A third blue color bead was inserted with 2-5 mm thread distance between them. This sequence was repeated three times to make a drug delivery device with twelve disks. A suture length of about 30 mm on both ends was used. For all 12 discs, a knot was placed at the entry point of the needle on the disk and also after the needle exit from the disk (knots on both ends). The knots before and after the disk were used to prevent movement/migration of disks after the device had been prepared. Terminal ends had about 30 mm of suture to be used for suturing the device to the tissue. In another variation of the above example, cylindrical disks with one or multiple holes (FIG. 1 B1, B2 and B3) were used to make the device. A knot that has a bigger size than the hole was used before and after the needle entry and exit is used to prevent disk migration.

The flexible space between each disk in some embodiments can be used to compact and reduce the overall dimension of the device.

### Example 3D4

### Threading Cylindrical Hydrogels Disks Through the Side.

### Use of Synthetic and Natural Biodegradable Hydrogels Disks

### Use of Tear Resistant Hydrogels (Fiber or Prosthetic Tissue Reinforced Hydrogels)

Crosslinked disks PEG35KUA hydrogel with Iodixanol and also with submucosa tissue was prepared by polymerizing the precursor solution on top of the perforated submucosa tissue in a mold. The perforated tissue is completely covered and immersed inside the precursor solution. The precursor is then crosslinked by exposure to light. The formed disk is removed from the mold. The disk thus formed has perforated submucosa tissue as an illustrative fiber reinforcement to provide tear resistance to the disks. Red colored gelatin methacrylate crosslinked gels prepared using as described before. The hydrogel disks were threaded as described before to make hydrogel-based drug delivery devices. The hydrogel disks can be partially or completely dehydrated before using in the device.

### Example 3D5

Threading cylindrical hydrogels disks through special features like hooks or loops on the disk/bead. Frozen solutions of PEG35KUA precursor with iodixanol in disk shape and in ring shape (without iodixanol) are placed next to each other so that at least one surface is in contact with each other. The frozen shapes are then irradiated to make a unibody material wherein the disk. The suture thread (708) is passed through the ring to make a threaded device.

### Example 3D6

Threading Cylindrical Hydrogels Disks from Two Sides.

### Preparation of Sheet Like Device Comprising Crosslinked Drug Delivery Disks.

Catgut suture (Ethicon, India size 4.0) was used to thread the 16 gelatin methacrylate photo-crosslinked gels (4 each from red, green, blue and purple color, each color representing a different drug/visualization agent). Suture needle was inserted in the blue disk via sideways (perpendicular to disk/bead axis) followed by red colored, green colored and purple colored disks. A 2 to 5 mm thread spacing was kept between the two disks. Four of such devices were made. A second suture thread was inserted in the blue disk perpendicular to the first insertion point and the process was continued for remaining four beads and continued until all 16 beads were threaded in 4 by 4 grid format wherein each disk was at the junction of thread intersection point (FIG. 2H and FIG. 5D). The matrix of disks formed using threaded suture can be implanted for local drug delivery.

In another modification of the above device, the matrix of the disk-shaped device as prepared was wrapped around a cylindrical pipe/rod and ends of the suture were tied to make a cylindrical device (FIG. 2K) which can be used in tubular shaped organs found in the body.

### Example 3D7

Preparation of Array from Sheet Material or Cylindrical Rods

### Crosslinked Hydrogel or Organogels Biodegradable Sheets and Rods

10 g of PEG10K-LACTATE-5-diacrylate (PEG10KL5A, Example 1), 3 g of PLGA (50:50, molecular weight 20000 g/mole as a carrier) and 0.15 g of bupivacaine base are dissolved in 50 ml tetrahydrofuran. After complete dissolution, 100 microliter photoinitiator is added and one drop of the solution is polymerized within 60 seconds. The solution is filled in glass petri dish until a solution height of about 1 mm is obtained. The solution is exposed to light io initiate polymerization and crosslinking. After effective crosslinking and gel formation, using a 2 mm diameter cutting die, several cylindrical pieces with 2 mm diameter and 1 mm height are cut from the cast organogel. The cut pieces are used as is or are dried form an array using gut suture as described before. In another variation of above example, the precursor PEG10KL5A solution as above is first filled in 2 mm diameter and 12 inches long glass/quartz tube and polymerized inside the tube by exposing to light. The crosslinked gels are then removed from the capillary and cut into 1 mm thick slices. The cut cylindrical disks are used in array formation in solvated or dry form as indicated above. Using similar procedure, crosslinked sheets or rods are prepared from crosslinking material made by precursors comprising electrophilic and nucleophilic groups via condensation polymerization as described earlier section. Protein solution such as albumin, gelatin or collagen crosslinked using a crosslinker such as glutaraldehyde also can be used in making rods or sheets.

### Example 3E

### Drug Delivery Devices Made by Casting Precursor Solution Inside a Thread.

Precursor solutions with bupivacaine, Iodixanol and with biodegradable carrier (PLGA) with no drug or visualization agent were prepared according to method described before. A polypropylene mold with six 3 mm diameter and 1 mm depth cylindrical cavities with 8 mm distance between them (center to center) was used. Catgut suture (Ethicon, India size 4.0) was first applied on the mold cavities covering all cavities. Care was taken to ensure that thread followed cavity contours and an adhesive tape was used to immobilize the thread. Three precursor solutions were filled in the mold cavities sequentially and completely. Care was taken that the thread was completely immersed in the precursor solution inside the cavity. The mold was then exposed to UV light to polymerize and crosslink the precursor solution. The crosslinked disks with embedded suture thread were taken out of the mold. The crosslinked device having three different compositions with embedded suture thread was obtained.

In another modification, a modified mold is used wherein a space (groove) was created on the mold surface and was used for casting as described. The groove was used to hold suture thread in the mold (FIG. 3A).

In another modification, six 10 mm long suture threads were cut. The suture threads were arranged in a rectangular grid (1 cm by 1 cm grid size). At each junction, a knot was used to make a 6 by 6, 1 cm apart grid. The grid was placed inside the mold with 3 mm diameter and 1 mm depth cavities. The mold cavities had an identical pattern as suture grid. The suture grid was placed inside the mold such that the thread intersection was at the center of the cavity. The three precursor solutions, as described above were then added in a sequence in the mold cavity such that the suture grid junction is completely immersed inside the precursor solution. The solutions were then exposed to long UV light for 5 minutes to effectively polymerize and crosslink the solutions. The polymerized disks were then lifted from the mold. A 6 by 6 suture thread matrix wherein at each junction of the grid, a crosslinked disk (similar to shown in FIG. 3 E1) was obtained. In another embodiment, a 36-cavity mold in a grid pattern with grooves for suture thread (FIG. 3 A1) was used to obtain single or multi-compositional connected beads as described above.

In another modification of above embodiment, instead of suture, a perforated submucosa tissue was used to create a grid like pattern (6 by 6 matrix, fiber replaced with submucosa tissue) was used and the disks were cast on the junction of the tissue grid pattern. In another modification, a biostable fiber matrix with biostable hydrogel disks are prepared using an example as above.

This disk is loaded clinical diagnostics agents where each hydrogel in the matrix has different composition designed for a different clinical diagnostics test. Each disk in the array represents a separate clinical test. Such array can be used to analyze 2, 3, 4 or several more clinical test simultaneously.

### Example 3F1

### Drug Delivery Devices Made by Casting Precursor Solution on Top of Coated Thread or Filament.

A 3 mm wide, 20 cm long sheep submucosa tissue was cut. The strip was coated with gelatin methacrylate precursor solution with photoinitiator and then placed in the refrigerator to freeze the coating. Gelatin methacrylate precursor solutions with red, green and blue colors as described before were cooled inside the 3 cm diameter and 1 mm height mold cavities until frozen. The frozen precursor disks were taken out of the mold and placed on top of the frozen coated strip at a distance of about 10 mm in a sequence. Care was taken that the disks and coating on the tissue strip remain frozen during the stacking process. The arranged disks were exposed to long UV light for 5 minutes to initiate polymerization of precursor disks and coating. The crosslinking occurs in the disks as well as between disk and coating forming a unibody structure. After the crosslinking, the submucosa strip with a disk attached to one of its surfaces was immersed in water at room temperature. The disks maintained their shape and size without dissolution and without detaching from the tissue surface indicating firm attachment of disks to the tissue strip surface.

In another modification of the above example, the frozen disks were arranged on the tissue strip without leaving a gap between them (closely packed arrangement) to produce crosslinked disks with little or no gap between the disks.

In another modification of the above example, disks from the above example were replaced with frozen drug/visualization agent loaded with microspheres of desired size and shape. The frozen microspheres were arranged in a matrix format and then crosslinked with precursor coated tissue to form a composite material wherein microspheres embedded disk were attached to the tissue.

### Example 3F2

### Drug Delivery Devices Comprising Multiple Microneedle Arrays Attached to Thread/Filament or Strip.

20 percent gelatin methacrylate solution was prepared by dissolving 0.201 g dry gelatin methacrylate in 1 ml distilled water. 10 microliters of photoinitiator solution were added to it and one drop of the solution tested for gel formation under UV light. The solution formed a gel within 60 seconds. The above solution was divided into three parts. One part was colored using a red ink and another was colored using a green ink and the third was left as is (uncolored and plain).

Plain solution was then poured into the silicon mold with cavities in the shape of a microneedle array. The mold was centrifuged to remove air bubbles and then frozen around 20 degrees C. The array was then removed from the mold and then placed on a gelatin methacrylate coated strip similar to prepared as reported in previous example (temperature−20 degree C., under frozen conditions). Frozen microneedle arrays with red and green needles were also prepared and then placed on top of the strip. The entire assembly was exposed to long UV light in frozen condition so that gelatin methacrylate microneedle blocks and tissue precursor coating were polymerized and crosslinked. The tissue strip with multiple microneedle devices attached was incubated in water for 5 minutes. None of the blocks detached from the strip indicating successful attachment of the array to the strip. The strip was removed and lyophilized.

### Example 3G

### Array Devices Comprising Live Cells

All experiments were conducted in sterile conditions in a sterile hood. Chinese hamster ovary (CHO) cells (supplied by ATCC, CHO-K1 (ATCC® CRL-9618) or human foreskin fibroblasts (HFF) were thawed to 37 degree C. and transferred to a 75-centimeter square tissue culture flask containing 20 ml of ATCC formulated F-12K Medium with fetal bovine serum (final concentration of 10%). The cell lines were used as an illustrative mammalian cell line. The HFF cells were incubated at 37° C. in a suitable incubator that provided 5% CO2 in the air atmosphere. The medium was changed daily. After 2-3 days and reaching full confluence, the cell culture medium was removed and cells were rinsed with 0.25% trypsin, 0.03% EDTA solution. An additional 1 to 2 mL of trypsin-EDTA solution was added and was incubated at 37 degree C. until the cells detached from the flask surface. The cells were centrifuged, the supernatant was removed and cells were resuspended in 5 ml cell culture media. 1 g of gelatin methacrylate was dissolved in 9 g of cell culture medium and 30 microliter of initiator solution (IS) was added. The solution was sterile filtered. 1 ml of gelatin methacrylate sterile solution and 0.1 ml cell suspension were mixed and viability of the cell was measured using standard live-dead cell assay. The solution was added in a mold cavity (3 mm diameter and 1 mm depth) and exposed to long UV light for 60 seconds. The polymerized gel was removed from the mold and viability of cells was checked again. Viability of cells should be greater than 50 percent preferably 70 percent before proceeding to the next step. Several such disks with live cells were made. The disks were then threaded to form a device as described before with live cells which could be used for therapeutic use. The device was attached to the body or organ or a surgical site using a suture.

One or several hundreds or thousands of such devices with live cells could be fitted in a bioreactor or microfluidic array in desired 2-dimensional or 3-dimensional arrangement to produce therapeutics drugs or other commercially useful chemical compounds.

In another modification of the above example, PEG20KUA was used in place of gelatin methacrylate and cells were replaced with a commercially useful enzyme. PEG20KUA macromonomer and lipase enzyme were dissolved in TRIS buffer (lipase concentration 1 mg/ml, 100 mM TRIS buffer and 1 mM EDTA, pH 8) and polymerized to form disks as described above. The disks were then threaded and used in a bioreactor for enzyme assisted biochemical reactor to produce desirable compounds catalyzed by encapsulated enzymes in the disks.

### Example 4

### Inter Connected Superabsorbent Biostable Hydrogels or Organogels.

### Interconnected Superabsorbent Hydrogels.

1 g of acrylic acid and 1.2 g of water were put into a 25 ml beaker and purged with nitrogen; 0.83 g of aqueous 45% sodium hydroxide was weighed separately and added slowly into the aqueous acrylic acid solution under ice cooling conditions for neutralization. In a 10 ml glass vial 0.102 g of PEG35KUA was dissolved in 1 ml of sodium acrylate solution followed by 20 microliter of photo-initiator and one drop of the solution was polymerized under UV. The solution formed a hydrogel upon exposure to UV light. Polypropylene sheet with several 3 mm and 1 mm dip cavities were lined with a cotton thread and PEG35KUA-acrylic acid solution was added in the mold cavity until all cavities were filled completely. The solution was exposed to liquid nitrogen to flash freeze and then exposed to long UV light for 10 minutes under frozen condition (around −20 to −5 degree C.). The crosslinked gel was removed from the mold, washed with water to remove unreacted monomer and then dried. The PEG35KUA-acrylic acid sodium salt hydrogels were interconnected by cotton fiber. Using a similar procedure, the polyacrylic acid gels were made using interconnected cotton threads in a mesh format and the gels were placed at the junction of two fiber intersections. In the third variation, a woven/knitted cotton fabric was used to make several gels separated by about 3-10 mm distance.

### Example 5

Implantable arrays with disks with drugs that are encapsulated in a drug carrier.

Biodegradable hydrogel disks with rifampin encapsulated biodegradable microspheres.

### Example 5A

### Preparation of Drug Encapsulated Microspheres.

Rifampin encapsulated PLGA microspheres (size 10-50 microns, rifampin loading 10 percent relative to PLGA) were prepared by either spray drying or via emulsion methods as described in related patent application (US 20190046479) or from the literature. Briefly, rifampin, PLGA were mixed in dichloromethane and the mixture was spray dried to produce rifampin encapsulated microspheres. The microspheres were vacuum dried to remove the solvent.

Alternatively composite microspheres with rifampin also can be used as described in this invention.

### Example 5B

Incorporation of drug encapsulated microspheres in hydrogel disks.

1 g of PEG10K-LACTATE-5-diacrylate (PEG10KL5A, Example 1) was dissolved in 5 ml PBS (pH 7.4). 10 microliters photoinitiator solution was added and one drop the solution was polymerized within 60 seconds upon exposure to long UV light. 1 ml of the above solution was mixed with rifampin 0.2 g of rifampin encapsulated microspheres prepared as above. The suspension is filled in a polypropylene mold with 3 mm diameter and 1 mm depth and exposed to long UV light for 5 minutes to polymerize and crosslink monomer solution to form about 3 mm diameter and 1 mm height disk/cylinders. The biodegradable hydrogel disks containing rifampin encapsulated microspheres were taken out from the mold and lyophilized. 12 lyophilized disks were threaded with 0 size synthetic polymer suture (VICRYL, polyglactin 910 Suture) and used in making surgical array as described in this invention. Alternatively, the monomer solution with microspheres can be cast on the suture or thread to make an array device.

The device upon implantation releases rifampin from the microspheres in a controlled manner and hydrogel for local antibiotic therapy. Using a similar procedure as above, drug rifampin was replaced with bupivacaine to make an array device.

### Example 5C

Preparation of drug loaded organogel or hydrogel disks with drug carrier. The drug carrier is organic solvent soluble biodegradable polymer, biodegradable thermosensitive or pH sensitive polymer or neat liquid carrier.

### Example 5C-1

### Disks Containing Organic Solvent Soluble Biodegradable Polymer as a Carrier.

1 g of PEG10K-LACTATE-5-diacrylate (PEG10KL5A, Example 1), 0.3 g of PLGA (50:50, molecular weight 20000 g/mole) and 0.015 g of bupivacaine base were dissolved in 5 ml dimethyl sulfoxide (DMSO). After complete dissolution, 10 microliter photoinitiator was added and one drop of the solution was polymerized within 60 seconds. The solution was filled in a polypropylene mold with 3 mm diameter and 1 mm depth and exposed to long UV light for 5 minutes to polymerize and crosslink macromonomer solution to form 3 mm diameter and 1 mm height disk/cylinders. The biodegradable organogel disks containing bupivacaine base were taken out from the mold and solvent was removed by vacuum drying. The solvent removal precipitates the drug in crosslinked material as well as inside drug carrier PLGA in the disk. The precipitation of PLGA encapsulates the drug and is therefore released in a controlled manner as PLGA is degraded inside the crosslinked hydrogel via hydrolysis. 12 lyophilized disks were threaded with 4.0 size synthetic polymer suture and used in making surgical arrays as described in this invention.

### Example 5C-2

### Disks Containing Thermosensitive Biodegradable Polymers as a Carrier.

### Aqueous Solution Polymerization of Biodegradable Macromonomer and PEG-Polylactone or Thermosensitive Polymer as Drug Carriers in Aqueous Solution.

1 g of PEG10K-LACTATE-5-diacrylate (PEG10KL5A), 1 g PEG 2000-lactide copolymer thermosensitive biodegradable carrier (PEG2KL-1-2) (Example 1), and 0.015 g of bupivacaine hydrochloride were dissolved in 5 ml cold PBS (0-5 degree C.). After complete dissolution, 10 microliter photoinitiator was added and one drop of the solution was polymerized within 60 seconds. The solution was then filled in a polypropylene mold with 3 mm diameter and 1 mm depth and exposed to long UV light for 5 minutes to polymerize and crosslink macromonomer solution to form 3 mm diameter and 1 mm height disk/cylinders. The biodegradable hydrogel disks containing bupivacaine and thermosensitive biodegradable PEG-polylactone carrier (PEG2KL-1-2) were taken out from the mold. The drug is entrapped in crosslinked hydrogel as well as in the thermosensitive biodegradable carrier (PEG2KL-1-2). Depending on the temperature, (0-40 degree C.), the thermosensitive polymer will be in solution phase or thermosensitive gel phase. At body temperature, 37 degree C., the polymer exists as a thermosensitive gel. 12 disks were threaded with 4.0 size synthetic polymer suture and used in making surgical arrays as described in this invention.

### Example 5C-3

### Disks Containing Sucrose Acetate Isobutyrate as Liquid a Carrier.

1 g of PEG10K-LACTATE-5-diacrylate (PEG10KL5A, Example 1), 0.3 g sucrose acetate isobutyrate and 0.015 g of bupivacaine base were dissolved in 5 ml dimethyl sulfoxide (DMSO). After complete dissolution, 10 microliter photoinitiator was added and one drop of the solution was polymerized within 60 seconds. The solution was filled in a polypropylene mold with 3 mm diameter and 1 mm depth and exposed to long UV light for 5 minutes to polymerize and crosslink macromonomer solution to form 3 mm diameter and 1 mm height disk/cylinders. The biodegradable organogel disks containing bupivacaine base and sucrose acetate isobutyrate as a liquid carrier were taken out from the mold and solvent was removed by vacuum drying.

The drug was entrapped in crosslinked hydrogel as well as in liquid carrier sucrose acetate. 12 such disks were threaded with 4.0 size synthetic polymer suture and used in making surgical arrays as described in this invention. The array has disks containing liquid carriers from which drug is released in a controlled manner.

### Example 6

### The Use of Crosslinkers to Make Hydrogels and Organogels.

### Preparation of Array Elements/Disks Using Albumin as a Model Protein and Glutaraldehyde as a Model Crosslinker.

### Example 6A

A 10 percent solution of bovine serum albumin in PBS (pH 7.4). Glutaraldehyde stock solution purchased from the manufacturer is diluted in PBS to form a 0.2 percent solution. The solutions were loaded in a double-barreled syringe (Baxter: Deerfield, IL). A first syringe contained 1 mL albumin solution and the second syringe contained 1 ml glutaraldehyde solution. Both solutions were mixed in equal volume in the dispensing tip and dispensed the solution in silicone rubber cavity mold (2 mm diameter, 1 mm height) with fiber. Alternatively, solutions can be added in the mold in sequence and mixed. The crosslinked albumin gels were removed and used to make array devices as described before. Alternatively, the solutions can be dispensed on top of suture thread leaving a gap of 1 cm between dispensing solution droplets. The solutions are allowed to cure and the array device with crosslinked albumin disks is removed from the mold.

### Example 6B

The aldehyde solution of the previous example (14A) is replaced with 0.2 percent solution 4 arm polyethylene glycol with molecular weight 10000 g/mole glutarate ester terminated with n-hydroxysuccinimide (PEG10K4ARM glutarate NHS ester) in PBS. The disks produced as above are crosslinked with PEG10K4ARM crosslinker. Alternatively, solutions can be also added in the mold in sequence and mixed.

### Example 6C

### Use of Frozen Protein Solution to Make Crosslinked Gel.

Several drops (10 microliter) of 10 percent albumin solution in PBS are dispensed along the length of suture with 1 cm distance between each drop. The assembly is kept in the refrigerator until the albumin solution is frozen. The frozen albumin drops and suture are incubated in 0.2 percent glutaraldehyde solution (1 to 4 degree C.) until effective crosslinking of albumin. The crosslinked albumin array device is removed and washed with PBS several times to remove aldehyde solution and stored in the refrigerator.

The uncured precursor/s/macromonomer/s microspheres/microparticles as described in Example 5 and 6 can be arranged on a fiber or fiber mesh with a desired arrangement like shown in FIGS. 3E, 3E1, 2H, 2F, 2A and 2C) and then cured to produced interconnected microspheres/microparticles based devices as described in this invention. In the preferred mode, the uncured microspheres are in frozen or dry or substantially dry state. Alternatively, the liquid uncured precursor/s/macromonomer/s liquid/solution droplets can be deposited via inject printing process or droplet dispensers in a desired pattern on a fiber or thread or fiber mesh and then cured to prepare an interconnected microparticle array like devices.

### Example 7

### Example 7A

### The Use of Non-Crosslinked Hydrogels and Organogels as Array Elements.

### Preparation of Array Elements/Disks Using Gelatin and Drug Loaded Microspheres.

Use of Natural Polymers. Non-Crosslinked Gelatin Hydrogel with Drug Encapsulated Microspheres.

1 g of gelatin is dissolved in 9 g of warm 60-70 degrees C. PBS buffer and cooled to around 40-50 degrees C. without gelation. 2000 mg of PLGA microspheres loaded with 10 percent rifampin is added to the 10 ml gelatin solution. The microsphere suspension is added in a circular silicone rubber mold 2 mm dia and 1 mm height and lyophilized. The dry gelatin disk with rifampin encapsulated microspheres is used for array preparation as discussed before. If desired, the lyophilized gelatin disks are exposed to 0.2 percent glutaraldehyde solution in cold (0-10 degree C.) PBS for about 30-360 minutes to crosslink the gelatin. The crosslinked gels are washed with PBS and lyophilized again.

In another variation, gelatin solution is substituted with collagen. 5 ml of collagen solution (4 mg/mL in a 0.2M acetic acid solution, pH 3.0) is added with 1 g of rifampin microspheres. The suspension is added in the mold and lyophilized. The non-crosslinked lyophilized collagen disks with encapsulated microspheres are used in array preparation.

In another variation, 5 ml of 0.2 percent hyaluronic acid sodium salt (Sigma molecular weight 1000000-1250000 g/mole) in PBS is mixed with 1 g of rifampin encapsulated microspheres as above and the suspension is added in the mold and lyophilized. If desired the suspension can be mixed with di or polyepoxide crosslinker like 1,4-butanediol diglycidyl ether or poly(ethylene glycol) diglycidyl ether and then added in the mold. After effective crosslinking, the disks are lyophilized. The lyophilized disks are removed and used for array preparation as described in this invention.

In another variation, 10 cm of gut suture surgical thread or porcine submucosa strip (3 mm wide, 10 cm long) is kept on a cold plate surface (around −20 degree C.). Warm gelatin suspension solution (around 40 degrees C.) with microspheres is loaded in a warm syringe and 0.020 ml of suspension is dispensed from the syringe with a needle on the suture or strip at a distance of about 10 mm from one of the terminal ends. About 8 drops are added with about 8-10 mm distance between each droplet. The gelatin is converted into a soft gel in contact with the cold plate. The suture/thread with embedded gelatin hydrogel is removed and lyophilized. The lyophilization produces an array device with non-crosslinked gelatin hydrogel with drug encapsulated microspheres embedded in suture/strip.

In another variation of this experiment, gelatin is substituted with fibrin glue or fibrin sealant components. Fibrin glue components (fibrinogen and thrombin) are mixed with drug encapsulated microspheres and the suspension is added in the mold and allowed to crosslink. The crosslinked disks are lyophilized and used in array preparation. Alternatively, the 0.02 ml fibrin glue suspension before crosslinking with microspheres is added on the tissue strip as above.

Several droplets are added at a distance of 8-10 mm and the solution is allowed to crosslink (fibrinogen, thrombin, factor 8 reaction). After effective crosslinking (fibrin clot formation) the array is lyophilized. The array has crosslinked fibrin glue elements with encapsulated microspheres at a distance of 8-10 mm on the tissue strip.

### Example 7B

### Use of Synthetic Non-Crosslinked Hydrogels.

Polyvinyl alcohol (PVA, Sigma, molecular weight 146,000-186,000, 99+% hydrolyzed) 10 g is dissolved in hot 100 g of water to make a homogeneous solution. In a 5 ml PVA solution at room temperature, 1000 mg of PLGA microspheres loaded with 10 percent rifampin are added and mixed thoroughly. The suspension is poured in the mold and dried or lyophilized. The lyophilized disks are used for array preparation.

In one variation, the PVA suspension in the mold is frozen at −20° C. for about 20-24 hours and then thawed at 25° C. for 6-8 hours. This freeze-thawing is repeated for five consecutive cycles and then lyophilized. The lyophilized disks are used for array preparation. The freeze thaw cycles are believed to introduce physical crosslinks in the PVA hydrogel.

### Example 7C

### Use of Organogels.

1 g of gelatin, 0.2 g of PLGA (molecular weight 15000-25000 g/mole) or 0.2 g PEG 2000-lactide copolymer thermosensitive biodegradable carrier (PEG2KL-1-2) optionally with drug and 80 ml of DMSO are mixed to form a homogeneous solution. The solution is added in the mold and solvent is removed by air drying followed by vacuum drying. The dried disks are then used for array preparation. The lyophilized gelatin disks with PLGA or PEG 2000-lactide copolymer as a drug carrier can be loaded with drug using solvent diffusion method as described before.

### Example 8

### Preparation of Porous Beads/Disks.

### Preparation Porous Synthetic Biodegradable Bead/Disks/Array Elements.

Preparation of Porous Structure by Salt Leaching and/or Lyophilization.

### Example 8A

### Preparation of Porous Poly(L-Lactide-Co-Caprolactone Copolymer

In 250 ml beaker, 1 g of Poly(L-lactide-co-caprolactone) copolymer (70/30; PURASORB PLC 7015 from PURAC Biochem, Netherlands) is dissolved in 20 ml 1,4-dioxane. After complete dissolution, 9 g sodium chloride (finely grounded and sieved, less than 20-50 micron particle size fraction) is added to the mixture and the suspension is vigorously stirred. The stirred liquid suspension is immediately poured into petri dish with a liquid/suspension height of about a 1-3 mm. The suspension is quickly frozen using liquid nitrogen. The solvent dioxane is removed by lyophilization around −12 to 10 degree C. Using a 1-3 mm diameter cookie cutter, 1-3 mm cylindrical pieces are cut from the freeze-dried polymer. The cut pieces are incubated in distilled water to 24-72 h to remove sodium chloride and to induce porosity. The leaching of sodium chloride leaves voids where sodium chloride was present. Suspension could be first poured into glass or polyethylene tube, 1 mm dia and frozen and lyophilized to produce a 1 mm dia freeze-dried solid. The freeze-dried solid is cut into 2 mm sections and the sections are subjected exposed to water for sufficient time to extract sodium chloride substantially or almost completely or completely. By changing the particle size of porogen (sodium chloride) and its weight percent in the above example, porous solids with different amount of porosity and size of pores can be obtained.

### Example 8B

### Preparation of Porous Structure by Leaching Out Organic Soluble Polymer.

### Preparation of Porous Hydrophilic Biodegradable Polymer (Enzymatically Degradable Polymer Collagen).

In a 250 ml beaker, 100 mg Type I collagen is dissolved in 10 ml 0.5 M acetic acid. To this solution, 10 mg of poly(ethyl methacrylate) spherical beads are added (from Polysciences, Particle size 140-220 microns). The suspension is stirred and quickly frozen using liquid nitrogen. The frozen solution is lyophilized to remove water and then transferred into 100 ml methanol. The poly(ethyl methacrylate) is extracted out in methanol for 7 days with fresh methanol exchanged every day. The dissolution of poly(ethyl methacrylate) by methanol creates porosity in the collagen matrix. In a similar embodiment, 10 g Poly (L-lactide) PURASORB PL 18 is completely dissolved in 100 ml dioxane. 5 g poly(ethyl methacrylate) microspheres are added to the mixture and the mixture is quickly frozen and then freeze dried at −5 degree C. The freeze-product is then subjected to extraction with methanol wherein only microspheres are soluble and Poly (L-lactide) is not soluble. The extracted microspheres leave behind spherical empty void which can be used to fill controlled drug delivery compositions.

### Example 8C

### Porosity Using Mechanical or Laser Drilling or Injection Molding.

A high molecular weight polycaprolactone or polylactic acid cylindrical rod (1 mm diameter) is extruded or injection molded from a laboratory based plastic processing extrusion machine or injection molding machine. Alternatively, the rod can be cast from a solution in a cylindrical mold and solvent is removed. The extruded/injection rod is cut to 1 mm dia and 2-4 mm length sections and the cut pieces are subjected to laser drilling/mechanical drilling. Several 25-100 microns diameter and 50 microns in depth holes are drilled on the polymer rod surface. UV, infrared or visible light based laser systems are used to drill a hole. Laser drilling induces holes/cavities on the surface which can act as porosity.

Alternatively small holes can be mechanically drilled on the rod surface to create artificial porosity. A micro-drill bit with 0.001 inch to 0.004 inch dia is used to drill holes.

### Example 8D

### Preparation of Porous Synthetic Biodegradable Crosslinked Organogels or Hydrogels Example 8D-1

### Synthetic Porous Hydrogels Created by Crosslinking of Biodegradable Macromonomers

1 g of PEG10K-LACTATE-5-diacrylate (PEG10KL5A) was dissolved in 5 ml toluene. After complete dissolution, 10 microliter photoinitiator was added and one drop of the solution was polymerized within 60 seconds. The solution was mixed with 1 g of sodium chloride finely ground powder (porogen) to make to make a suspension. After stirring, the solution was filled in a polypropylene/glass mold with 2 mm diameter and 1 mm depth and exposed to long UV light for 5 minutes to polymerize and crosslink macromonomer solution to form 2 mm diameter and 1 mm height disk/cylinders. The solvent was removed by air drying followed by vacuum drying. The dried disks were incubated in distilled water to swell the crosslinked polymer network and to leach out sodium chloride porogen. After complete or substantial removal of porogen, the crosslinked hydrogel disks were lyophilized. The lyophilization and sodium chloride removal produces porosity in the crosslinked polymer. The dried disks were used for drug infusion and to form an interconnected array device as disclosed in this invention.

In another modification of above example, 0.3 g of PLGA (50:50, molecular weight 20000 g/mole) was dissolved along with PEG10KL5A in dichloromethane and the mixture is crosslinked and treated with water to create a porous structure in crosslinked polymer and as well as in entrapped PLGA.

In a 10 ml glass vial, 0.2 g of PEG2KL-1-2 polymer (Example 1D, water soluble PEG-polylactone polymer) and 1 g of PEG10K-LACTATE-5-diacrylate (PEG10KL5A, Example 1) are dissolved in ice-cold 3.5 ml of PBS. To this solution, 1 g of poly(ethyl methacrylate) spherical beads (porogen from Polysciences, particle size 140-220 microns) and 50 microliter photoinitiator solution are added and the suspension is stirred well. The suspension is added in the mold and crosslinked as described above. The crosslinked disks are incubated in methanol to leach out spherical beads as porogen. The porous hydrogel is then dried and used to make array device.

### Example 8D-2

### Synthetic Porous Hydrogels Created by Crosslinking of Precursors Comprising Electrophilic and Electrophilic Groups

In a 15 ml glass vial, 1 g of 4 arm polyethylene glycol with molecular weight 10000 g/mole and terminal amino groups per arm (PEG10K4 ARM tetramine) was dissolved in 9 ml of PBS (pH 7.4). to this solution, 1 g of poly(ethyl methacrylate) spherical microspheres (particle size 140-220 microns) were added as porogen. In another 15 ml glass vial, 1 g of 4 arm polyethylene glycol with molecular weight 10000 g/mole glutarate ester terminated with n-hydroxysuccinimide (PEG10K4ARM glutarate NHS ester) was dissolved in 9 ml of PBS (pH 7.4). 0.5 ml of PEG10K4 ARM tetramine suspension and 0.5 ml of PEG10K4ARM glutarate NHS ester solutions were mixed in 5 ml glass vial and immediately loaded into several 3 mm diameter and 1 mm height cylindrical cavity molds. After complete crosslinking, the crosslinked disks were incubated in methanol to remove the porogen and lyophilized. The crosslinked biodegradable polymer disks created has 140-220 size pores that can be loaded in with drugs.

### Example 8E

Porous Protein Microspheres (Crosslinked and not-Crosslinked)

1 g of bovine albumin is dissolved in 9 ml PBS buffer pH 7.2. After dissolution, 1 g of calcium carbonate or magnesium carbonate fine powder (as a porogen, size less than 1 micron) is added to the albumin solution. The suspension is loaded in a syringe with 20-to-32-gauge needle. The suspension is pushed out from the syringe and about 0.5 to 2 mm droplets (depending on the needle size used) are collected in the liquid nitrogen. The liquid nitrogen is evaporated and the frozen droplets are collected and lyophilized. The lyophilized albumin has porosity created by removal of free water. The lyophilized microspheres are exposed to acidic solution in 50 percent ethanol which is a non-solvent for albumin. Exposure of these microspheres to acidic solution dissolves carbonate salts in the microspheres leaving behind the empty space (porosity) created by carbonate salts. The lyophilized albumin particles are collected and stored under refrigeration.

In another variation of this method, frozen albumin solution droplets in liquid nitrogen were exposed to 0.2 percent cold (5-15 degree C.) glutaraldehyde solution to crosslink the albumin for 6 hours. After 6 hours, the crosslinked microspheres are separated and then exposed to dilute hydrochloric acid (0.05 molar in 50 percent ethanol) or acetic acid solution to remove the porogen carbonate salt. The crosslinked porous spheres are washed with distilled water several times to remove the acid and salts and then lyophilized. The removal of carbonate salts by acid wash leaves behind the empty space vacated by carbonate salts in crosslinked albumin microspheres which serve as a porosity. Additionally, lyophilization also removes free water from crosslinked albumin to induce additional porosity in the crosslinked microspheres. The amount of water and salt in the crosslinked microsphere preparation as mentioned above is used to control desired porosity in the microspheres.

### Example 9

### Infusing Controlled Drug Delivery Composition in the Pores of Array Elements Delivery Compositions.

### Drug Infused Porous Array Elements.

### Example 9A

### Drug Infusion Using Organic Solvents.

Deposition of drug crystals in microparticles without drug carrier. 100 mg of chlorhexidine diacetate salt hydrate bis(biguanide) (from Sigma, a model drug with low water solubility) is dissolved in 2 ml ethanol. 10 porous non crosslinked polymer beads/particles (Poly(L-lactide-co-caprolactone copolymer, Example 8A). Ethanol is non-solvent for Poly(L-lactide-co-caprolactone copolymer but solvent for chlorhexidine diacetate.

The disks are incubated for 1 hour. Particles are removed from the drug solution and the solvent is removed by air-drying followed by vacuum drying. Removal of solvent leaves behind the drug crystals in the porous particle. About 5 particles are placed in the 3 ml PBS solution at 37 degree C. to monitor the drug release under sync conditions. The drug is released due to slow dissolution of the drug in the aqueous solution. The PBS is exchanged every day and the drug release is monitored over a period 1-90 day, each time changing replacing the PBS solution. The drug concentration is monitored either by UV spectrophotometer or HPLC. It is important that the solvent chosen to load the drug does not dissolve the porous implant or affects its porous structure or structural integrity. The artisans can find solvent and non-solvent lists for various polymers in the Polymer Handbook (editor J. Brandrup, Wiley-Interscience) or other scientific literature or by doing laboratory experiments. This method is suitable for those drugs that have low solubility in water or physiological fluids. Drug solubility is generally is less than 1 g/100 g of water, preferably 0.5 g/100 g of water is highly desired.

### Example 9B

Filling the pores using synthetic biodegradable polyester or polylactone as a drug carrier In a glass vial, 10 ml tetrahydrofuran or 1,4-dioxane, 900 mg g Poly (PLGA, lactide-co-glycolide) (lactide:glycolide (50:50), molecular weight 30000 to 60000 g/mole.) and 90 mg (approximately 10 percent loading relative to weight of polymer) Latanoprost are mixed until homogeneous solution. A porous collagen implant made porous as above or commercial collagen punctal plug from Odyssey Medical, Inc (size 0.4 mm dia×2 mm length) is used. Two dry collagen porous implants are added in the THF drug solution and after complete penetration of the solution into the pores, the implants are removed from the solution, air dried followed by vacuum drying. If dioxane is used as a solvent; the implant can be frozen in liquid nitrogen immediately after complete penetration of the solution and then dioxane is removed by freeze-drying or lyophilization at cold temperatures (−20 to −5 degree C.). The drug loaded particles are used for array preparation as indicated above.

### Example 9C

### Filling of Drugs in the Pores of the Porous Implant Using a Water Soluble Biodegradable Drug Carrier

1 g of chlorhexidine diacetate salt hydrate bis(biguanide, 3 g Pluronic F127 and 4 ml methanol are transferred to a 15 ml glass vial. The solution stirred overnight until all the components are soluble. Expanded polytetrafluoroethylene (EPTFE, approximately 1 mm thick, 0.5 mm dia punched out from vascular graft, 6 mm dia by 30 cm length, C. R. Bard) is used as a porous biostable material. The Porous EPTFE is incubated in the methanol solution for 30 minutes. The EPTFE beads have porosity in the form of nodes and fiber structure with internodal distance from 10-100 microns). Free space in the implant is occupied by the drug solution. The implant is removed, air-dried and used in array preparation. The Pluronic F127 polymer dissolves in the aqueous or physiological environment releasing the model drug in a controlled manner. In another variation Pluronic F127 is substituted with thermosensitive biodegradable carrier (PEG2KL-1-2) using THF as a solvent.

### Example 10

### Example 10A

Drug Infused Array Device where Array Elements are Porous Materials and Pores are Partially or Completely Filled with Drug Delivery Composition.

### Mechanical Threading of Porous Particles Comprising Drug.

Array Device where Drug is Present Only Array Elements and not on Thread.

A 12 cm long gut suture thread with needle (USP size 5-0) is used to interconnect 10 porous beads prepared per Example 9 with drug. A 5 mm distance between each bead with knots on both sides was used. A 5 cm for free thread on both ends was left. The array prepared as above has drug infused in porous particles and not on thread used (FIG. 8G1).

### Example 10B

Array Device where Drug is Present Only Array Elements as Well as Thread.
Drug Infused Array where Whole Array Device is Incubated in the Drug Solution.

A 12 cm long gut suture thread with needle (USP size 5-0) is used to interconnect 10 porous beads prepared as shown above without a drug. A 5 mm distance between each bead with knots on both sides was used. A 5 cm for free thread on both ends was left. The interconnected device was then used for drug infusion. In a glass vial, 10 ml tetrahydrofuran or 1,4-dioxane, 900 mg g Poly (PLGA, lactide-co-glycolide) (lactide:glycolide (50:50), molecular weight 30000 to 60000 g/mole.) and 90 mg (approximately 10 percent loading relative to weight of PLGA polymer) rifampin are mixed until homogeneous solution. The array device including its thread and array elements are incubated for 30 minutes. After infusion of sufficient quantity of drug, the array is taken out and air dried followed by vacuum dried. Solvent removal keeps thin drug coating on gut suture. PLGA and rifampin is also coated/deposited in the pores of array elements (FIG. 8G). The device is sterilized with ethylene oxide and packaged. The device has drug rifampin present on thread as well as in porous array element.

### Example 11

### Preparation of Calcium Alginate Gel Particles

1 gram of Sigma-Aldrich grade sodium alginate suitable for microorganism immobilization and 50 milliliters of calcium ion-free HEPES buffer solution (pH 7.2) are added to a 250 ml beaker equipped with a magnetic stirrer. The mixture is heated to 45 degrees Celsius and stirred until complete dissolution occurs. Subsequently, the alginate solution is transferred into a 10-milliliter syringe fitted with a 31 G needle. The solution is manually extruded as liquid droplets and filled into the mold cavity with thread. The solution is frozen in refrigerator along with thread. The solidified frozen solution is then exposed to 300-milliliter calcium chloride (100 mM) and sodium chloride (145 mM) solution and the entire assembly is warmed to ambient temperature, resulting in the formation of calcium alginate gel particles that are embedded in thread. These particles are separated, used, and immediately utilized in the subsequent step. Another modification of this examples uses live cell suspension that is mixed with alginate solution along with dimethylsulfoxide as cryogenic protective agent. The solution is cooled in a controlled to maintain viability and then exposed to calcium solution for ionic crosslinking. Yet in another modification of above example, poly(allyl amine) hydrochloride (PAA-HCl) at a concentration of approximately 0.1-1 mg/mL in an appropriate buffer (pH 7-8) is prepared. The calcium crosslinked alginate beads are then exposed to the PAA-HCl solution for 30 minutes with gentle stirring. The ionic bonding between polyamine and calcium alginate further stabilizes the beads.

If necessary, additional layers of calcium alginate and PAA-HCl are added by repeating the procedure as outlined above.

### Example 12

### Preparation of Calcium Alginate Gel Particles

Array with Some or all Elements are Magnetic Array

In a 10 ml glass vial, 0.1 gram of PEG20KUA macromonomer is dissolved in 1 ml of DMSO. To this solution, 4 μl of initiator stock solution (ISS) is added. 100 mg of magnetic ferrite powder is subsequently added to the suspension, and the mixture is vigorously stirred. The suspension is polymerized in a mold along with a thread to form an array. The array is subsequently exposed to the magnetic field of a laboratory magnet. The array elements are attracted to the magnet and adhere to it, indicating the induction of magnetic properties in the array elements. In a modified version of the above example, magnetic iron oxide is first synthesized using a literature procedure and subsequently utilized in array formation as indicated above.

### REFERENCES

All references recited herein are incorporated herein by specific reference in their entirety.
1. J. Brandrup et. al., “Polymer Handbook”, John Wiley & Sons (2003).

### CITED US PATENTS AND PATENT APPLICATIONS

U.S. Pat. Nos. 5,410,016; 5,529,914; 5,874,500; 6,004,573; 6,534,591; 6,566,406; U.S. Pat. Nos. 6,887,974; 7,009,034; 7,740,877; 7,790,141; 8,067,031; 6,201,065; 9,023,379; 6,387,977; 9,789,073; 9,498,557; 6,201,065; 5,741,323; 8,506,856; 9,072,814; 7,790,141; 7,916,856; U.S. Pat. Nos. 9,074,865; 10,622,452; US Patent Application 2014/0256617; US Patent Application 2019/0046479; US Patent Application 2016/0166504; US Patent Application 2005/0069572; US Patent Application 2015/0060699; US Patent Application 2022/0118416; US Patent Application 2024/0399329; US Patent Application 2024/0252452; and US Patent Application 2008/0058787.

1. An implantable biodegradable array device, comprising:
an array comprising a plurality of array elements interconnected by a biodegradable thread, wherein:
the array includes three or more array elements;
at least 60% of the array elements are separated by substantially equal thread lengths;
the biodegradable thread comprises at least one terminal portion extending 2 mm or more beyond the connected array elements; and
both the biodegradable thread and the array elements are composed of biodegradable materials configured for degradation in a biological environment.

2. The array device of claim 1, wherein the array elements are approximately the same size.

3. The array device of claim 2, wherein the array elements are approximately the same shape, and the shape is symmetrical, non-symmetrical, or a combination thereof,
wherein optionally the shape is cylindrical, cubic, cuboid, conical, spherical, elliptical, rectangular prismatic, triangular prismatic, hexagonal prismatic, square pyramidal, rectangular pyramidal, triangular pyramidal, hexagonal pyramidal, ring-shaped, toroidal, or combinations thereof.

4. The array device of claim 3, wherein more than 50 percent of the array elements are fixed or immobilized on the thread to resist migration along a thread length.

5. The array device of claim 4, wherein each array element is embedded within the thread.

6. The array device of claim 5, wherein the thread and array elements are biodegradable, and the biodegradation occurs through an enzymatic process or hydrolysis.

7. The array device of claim 6, wherein the thread is selected from a braided, twisted, or monofilament structure.

8. The array device of claim 7, wherein the thread is composed of a synthetic biodegradable polymer or is derived from animal tissue.

9. The array device of claim 8, wherein the synthetic biodegradable polymer is selected from:
polymers, dendrimers, copolymers or oligomers of glycolide, dl-lactide, d-lactide, 1-lactide, caprolactone, dioxanone and trimethylene carbonate;
degradable polyurethanes;
degradable polyurethanes made by block copolymers of degradable polylactone, polycaprolactone, polycarbonate;
poly(hexamethylene carbonate);
tyrosine-derived polycarbonates;
tyrosine-derived polyacrylates;
polyamides;
polyesters;
polypeptides;
polyhydroxy acids;
polylactic acid;
polyglycolic acid;
polyanhydrides;
polylactones;
polyhydroxyalkanoates, which are polyesters produced by microorganisms;
poly(3-hydroxybutyrate);
3-hydroxyvalerate;
4-hydroxybutyrate;
3-hydroxyhexanoate,
3-hydroxyoctanoate;
polyethylene glycol-polyhydroxy acid;
polyethylene glycol-polylacone copolymers (PEG-PL copolymers);
polyvinyl alcohol-co-polylactide copolymers;
derivatives of cellulose;
collagen or modified collagen derivatives;
gelatin;
albumin or cross linked albumin;
fibrinogen;
keratin;
starch;
hyaluronic acid;
dextran; and
combinations thereof.

10. The array device of claim 8, wherein the animal tissue is derived from the submucosal layer of sheep intestines, goat intestine, pig intestine or the serosal layer of bovine intestines.

11. The array device of claim 7, wherein the array device comprises at least one drug or visualization agent.

12. The array device of claim 11, wherein the drug is present on the thread, the array elements, or both, and is configured to release 90% of its content in a controlled manner over a period of 2 hours to 6 months.

13. The array device of claim 12, wherein the drug is selected from the group consisting anti-infectives, antibiotics, antifungals, antivirals, antibacterials, antipruritics, anticancer agents, antipsychotics, cholesterol- or lipid-lowering agents, cell cycle inhibitors, antiparkinsonism drugs, HMG-CoA reductase inhibitors, anti-restenosis agents, anti-inflammatory agents, antiasthmatic agents, anthelmintics, glucagon-like peptide 1 (GLP-1), immunosuppressives, muscle relaxants, antidiuretics, vasodilators, nitric oxide and nitric oxide-releasing compounds, beta-blockers, hormones, antidepressants, decongestants, calcium channel blockers, growth factors, bone growth factors, bone morphogenic proteins, wound healing agents, analgesics and analgesic combinations, local anesthetic agents, antihistamines, sedatives, angiogenesis modulators, tranquilizers, therapeutic cellular elements, mammalian cells, stem cells, cellular components or fragments, enzymes, DNA, RNA, mRNA, genes, and combinations thereof.

14. The array device of claim 11, wherein the visualization agent is selected from the group consisting of radio-opaque contrast agents, fluorescent dyes, and colored compositions approved for medical applications.

15. The array device of claim 1, wherein the average thread diameter is more than 10 microns.

16. A method for producing an implantable surgical array, comprising:
providing a plurality of biodegradable microparticles with an average diameter greater than or about 100 microns and with at least one hole with a diameter greater than or about 10 microns;
coating or embedding the biodegradable microparticles with one or more drugs;
providing a flexible thread that is biodegradable;
threading the biodegradable microparticles onto the flexible thread to form an array, wherein the biodegradable microparticles are spaced at predetermined intervals or arranged in direct contact with each other; and
securing the biodegradable microparticles in place on the thread by mechanically locking, embedding, or knotting to prevent migration along the thread.

17. The method of claim 16, further comprising spacing the biodegradable microparticles at least 60% are separated by substantially equal thread lengths.

18. The method of claim 16, further comprising forming the flexible thread to be braided, twisted, or monofilament.

19. The method of claim 16, further comprising forming the microparticles to have at least one of an approximate same size or approximate same shape.

20. The method of claim 16, further comprising forming the array so that the flexible thread comprises at least one terminal portion extending 2 mm or more beyond the connected biodegradable microparticles.

21. The method of claim 16, further comprising forming the thread so as to be composed of a synthetic biodegradable polymer or is derived from animal tissue.

22. The method of claim 21, wherein the synthetic biodegradable polymer is selected from:
polymers, dendrimers, copolymers or oligomers of glycolide, dl-lactide, d-lactide, 1-lactide, caprolactone, dioxanone and trimethylene carbonate;
degradable polyurethanes;
degradable polyurethanes made by block copolymers of degradable polylactone, polycaprolactone, polycarbonate;
poly(hexamethylene carbonate);
tyrosine-derived polycarbonates;
tyrosine-derived polyacrylates;
polyamides;
polyesters;
polypeptides;
polyhydroxy acids;
polylactic acid;
polyglycolic acid;
polyanhydrides;
polylactones;
polyhydroxyalkanoates, which are polyesters produced by microorganisms;
poly(3-hydroxybutyrate);
3-hydroxyvalerate;
4-hydroxybutyrate;
3-hydroxyhexanoate,
3-hydroxyoctanoate;
polyethylene glycol-polyhydroxy acid;
polyethylene glycol-polylacone copolymers (PEG-PL copolymers);
polyvinyl alcohol-co-polylactide copolymers;
derivatives of cellulose;
collagen or modified collagen derivatives;
gelatin;
albumin or cross linked albumin;
fibrinogen;
keratin;
starch;
hyaluronic acid;
dextran; and
combinations thereof.

23. The method of claim 21, wherein the animal tissue is derived from the submucosal layer of sheep intestines or the serosal layer of bovine intestines.

24. A method of implanting an array device, comprising:
providing an array device comprising a plurality of array elements interconnected by a biodegradable thread, wherein:
the array includes three or more array elements;
at least a portion of the array elements are separated by substantially equal thread lengths;
the biodegradable thread comprises at least one terminal portion extending 1 mm or more beyond the connected array elements; and
both the biodegradable thread and the array elements are composed of biodegradable materials configured for degradation in a biological environment; and
implanting the array device into a subject.

25. The method of claim 24, wherein at least one array element of the array includes a visualization agent or a drug.
